(19) United States
(12) Patent Application Publication
(10) Pub. No.: US 2022/0023607 A1
(43) Pub. Date: Jan. 27, 2022

(54) Pressure Waves for Vaccines, Genetic Modification and Medical Treatments

(71) Applicant: Sanuwave Inc., Suwanee, GA (US)

(72) Inventor: Iulian Cioanta, Milton, GA (US)

(21) Appl. No.: 17/380,791

(22) Filed: Jul. 20, 2021

Related U.S. Application Data
(60) Provisional application No. 63/054,491, filed on Jul. 21, 2020.

(51) Int. Cl. A61M 37/00

(52) U.S. Cl. CPC A61M 37/0092; A61M 2202/30; A61M 2037/0007

(57) ABSTRACT

Pressure waves are applied to intersect a focal volume or pressure field with a targeted tissue in conjunction with introducing a treatment agent into the targeted tissue to increase permeability of cells in the targeted tissue and cause more rapid absorption of the treatment agent in the targeted tissue.

### CROSS-REFERENCE TO RELATED APPLICATIONS

This application claims the benefit of priority of U.S. Provisional Application No. 63/054,491 filed Jul. 21, 2020, which is incorporated herein by reference in its entirety.

### BACKGROUND OF THE INVENTION

In the last decades, there were new types of viruses that produced severe infections in humans or seasonal re-occurrence of certain diseases as flu or influenzae. In this realm, the mouth, eyes, nasal cavity, throat, lungs, stomach, intestines are the most susceptible areas and organs to such infections, since there is a conduit or conduits linked to them that gives a direct pathways access for the external pathogens to penetrate inside the human or animal body.

Within the lungs, the bronchial tubes branch many times into thousands of smaller, thinner tubes called bronchioles. These tubes end in bunches of tiny round air sacs called alveoli. The alveoli are where oxygen and carbon dioxide are exchanged. The tissues of the respiratory tract are thin and delicate, and become thinnest at the surfaces of the alveoli, where gaseous exchange occurs. Small blood vessels called capillaries run along the walls of the air sacs. When air reaches the air sacs, oxygen passes through the air sac walls into the blood and in the capillaries. At the same time, a waste product, called carbon dioxide (CO₂) gas, moves from the capillaries into the air sacs. This process, called gas exchange, brings in oxygen for the body to use for vital functions and removes the CO₂. The airways and air sacs are elastic or stretchy. When a living organism breathe in, each air sac fills up with air, like a small balloon and when breathe out, the air sacs deflate and the air goes out. The body has a number of mechanisms, which protect these tissues and ensure that debris and bacteria do not reach them. Tiny hairs called cilia trap large pieces of debris and waft them out of the airways; the reflexes of sneezing and coughing help to expel particles from the respiratory system and the production of mucus keeps the tissues moist and helps to trap small particles of foreign matter such as dust particles, bacteria, and other inhaled debris. Mucus production in the airways is normal and it contains glycoproteins (or mucins), natural antibiotics, which help to destroy bacteria, as well as proteins derived from plasma, and products of cell death such as DNA fragments. Without the mucus, airways become dry and malfunction. In the cases of pathogens infections and some chronic diseases of the lung, the mucus is produced in excess and changes in nature. This results in the urge to cough and expectorate this mucus as sputum. Sputum production is associated with many lung diseases processes and in these cases, it may become infected, stained with blood or contain abnormal cells.

An acute respiratory tract infection or disease is usually caused by an infectious agent, as bacteria, viruses, or funguses. Lung acute responses can be produced also by irritant particles that are voluntary or accidentally ingested. Although the spectrum of symptoms of acute respiratory infection may vary, the onset of symptoms is typically rapid, ranging from hours to days after infection. Symptoms include fever, cough, sore throat, inflammation of the mucous membrane in the nose, shortness of breath, wheezing, or difficulty in breathing.

Bacteria can cause pneumonia or tuberculosis. The most common causes of bacterial lung infections in normal hosts include Streptococcus pneumoniae, Haemophilus species, Staphylococcus aureus and Mycobacterium tuberculosis.

Aspergillosis is infection, usually of the lungs, caused by the fungus Aspergillus. A ball of fungus fibers, blood clots, and white blood cells may form in the lungs or sinuses. People may have no symptoms or may cough up blood or have a fever, chest pain, and difficulty breathing.

For the respiratory system, the humanity witnessed severe viral infections (originated from viruses) as the Severe Acute Respiratory Syndrome (SARS-CoV) in 2003, porcine flu in 2009, Middle East Respiratory Syndrome (MERS) in 2015, and lately the Corona Virus Disease (COVID-19) in 2019-2021, which points out the human vulnerability against virulent respiratory viruses. The existing evidence on Corona viruses suggests that they may follow the pattern seen in influenza. These viruses have spike proteins or grabbers that hook onto host cells cleavage site that allows the virus to open and enter those cells. When harmful pathogens invade and start to reproduce, the immune system recognizes them by their shapes. The pathogens have antigens, which are special proteins that trigger an attack from the body's immune system. These antibodies attach to antigens on the pathogens and prevent pathogens from invading other cells. Antibodies signal other white blood cells, which kill and remove the pathogens. A specific shape of antibody is needed to be efficient against a certain pathogen. The human body has billions of white blood cells, each making its own special-shaped antibody. Only a few antibody shapes will be effective against a specific pathogen. It can take several days for the immune system to produce enough properly shaped antibodies to kill the invading pathogens. During that time, a fast-acting pathogen, which can replicate billions of copies of itself, is a critical health threat. In severe cases of COVID-19, patients experience pneumonia, which means their lungs begin to fill with pockets of pus or fluid. This leads to intense shortness of breath and painful coughing. In general, after such severe viral infections the lung is damaged and not enough oxygen is supplied to the rest of the body, respiratory failure could lead to organ failure and death.

These viral infections can produce fever, respiratory symptoms as dry cough and shortness of breath, myalgia or fatigue, weight loss, cell debris-filling bronchiolar lumen, alveolar collapse with hemorrhage, and radiological ground-glass lung opacities.

Besides the severe acute respiratory syndrome Corona virus (SARS-CoV or COVID-19), the most known viral pathogens that cause this disease include influenza virus, parainfluenza virus, rhinovirus, and respiratory syncytial virus (RSV).

Acute respiratory infections are the leading cause of morbidity and mortality from infectious disease worldwide, particularly affecting the youngest and oldest people, as shown by the recent COVID-19 global pandemic or by mixed viral-bacterial infections. Although the knowledge of transmission modes is ever-evolving, the current evidence indicates that the primary mode of transmission of most acute respiratory diseases is through droplets, direct contact (including hand contamination followed by self-inoculation) or infectious respiratory aerosols. In general, such infections can be contagious and spread rapidly.

With the increased prevalence of highly contagious diseases such as Hepatitis B and Acquired Immune Deficiency Syndrome (AIDS), efficient prophylactic treatments and effective preventive viral vaccination are needed.

In treating such afflictions produced by new pathogens, the immediate treatment approach is the use of local or systemic medication, drugs, antibiotics, antibodies cocktails, homeopathic agents, which are targeting the specific invading organism. This medication approach to the treatment is hindered by the location in the tissue (fibrous or scar tissue) of bacteria, viruses, funguses and other harmful micro-organisms that makes the drugs ineffective due to inflammation and poor oxygenation of the tissue, which prevents drug to reach the infected tissue, resistance for the specific drug, etc. Also, in the case of medication, although in general potent and with immediate impact, the biochemical resistance of bacteria and viruses to antimicrobial agents may occur by mutation, natural selection, transformation, transduction or conjugation, which produces antibiotic resistance. Bacteria initially sensitive to an antimicrobial agent may become resistant, and another antimicrobial agent must then be used. The global concerns for developing antimicrobial drug resistance and the need to develop more prudent and judicious use of drugs have caused the necessity of finding new approaches to treat infections that do not display these disadvantages.

Besides specialized drugs, the vaccines represent of a potent means to fight against viral infections. Global health authorities and vaccine developers are currently partnering to support the technology needed to produce vaccines and develop new methods of delivery that can produce an immediate and expedite reaction and protection against viral infections. Some approaches have been used before to create vaccines, but some are still quite new.

Live vaccines use a “weakened” (“attenuated”) form of the germ that causes a disease. A virus is conventionally weakened for a vaccine by being passed through animal or human cells until it picks up mutations that make it less able to cause disease. Practically, the genetic code is altered so that viral proteins are produced less efficiently, when the vaccine germs are infecting a normal living cell. This kind of vaccine prompts an immune response without causing disease. The term “attenuated” means that the vaccine's ability to cause disease has been reduced. Live vaccines are used to protect against measles, mumps, rubella, smallpox and chickenpox viruses. As a result, the infrastructure is in place to develop these kinds of vaccines. However, live virus vaccines often need extensive safety testing. Some live viruses can be transmitted to a person who isn't immunized. This is a concern for people who have weakened immune systems.

Other category are the “inactivated” vaccines where the virus is rendered non-infectious or killed or inactive, by using chemicals, such as formaldehyde, or heat. This kind of vaccine causes an immune response but not infection when they go inside a live cell (require the penetration of the normal cells). Inactivated vaccines are used to prevent the flu, hepatitis A, and rabies. However, inactivated vaccines may not provide protection that is as strong as that produced by live vaccines. Making them, requires starting with large quantities of infectious virus. This type of vaccine often requires multiple doses, followed by booster doses, to provide long-term immunity. Producing these types of vaccines might require the handling of large amounts of the infectious virus.

There are also the “viral-vector” vaccines. Replicating viral vectors, such as weakened measles or Ebola viruses, can replicate within the cells. Such vaccines tend to be safe and provoke a strong immune response. However, existing immunity to the vector could blunt the vaccine's effectiveness.

Another category is the “non-replicating viral vectors” vaccines that use for the example the Adenoviruses. For these vaccines a modified version of an Adenovirus is used which can enter human cells but not replicate inside. The use of Adenoviruses has a long history in gene therapy. Booster shoats can be needed to induce long-lasting immunity.

A new type of vaccines is the “genetically engineered” vaccines that use genetically engineered ribonucleic acid (RNA) or deoxyribonucleic acid (DNA) that have instructions for making copies of the spike (S) protein. The RNA principal role is to act as a messenger carrying instructions from DNA for controlling the synthesis of proteins, although in some viruses, the RNA rather than DNA carries the genetic information. The DNA is a self-replicating material, which is present in nearly all living organisms as the main constituent of chromosomes. The RNA or DNA are carriers of genetic information. For these types of vaccines, the aiming is to use genetic instructions (in the form of DNA or RNA) for a Corona virus protein that prompts an immune response. The DNA or RNA is inserted into human cells, which then churn out copies of the virus protein. Most of these vaccines encode the virus's spike protein. These copies prompt an immune response to the virus and also with side effects/reactions as injection site pain or tenderness or systemic reactions such as fever and malaise. With this approach, no infectious virus needs to be handled.

Another option to manufacture vaccines are the “protein-based” vaccines. Fragments of proteins or protein shells that mimic the Corona virus's outer coat are used. For COVID-19, the virus's spike (S) protein is called the receptor binding domain. To work, these vaccines might require adjuvants, as immune-stimulating molecules delivered alongside the vaccine, as well as multiple doses.

Another approach is to produce “empty virus shells” to mimic the virus structure, but they are not infectious because they lack genetic material. Although they can trigger a strong immune response, they can be difficult to manufacture. These vaccines do not need to put any material into a living cell, but rather make these proteins available to be discovered by the body's immune cells that will generate in time the appropriate immune response.

The development of vaccines can take years. This is especially true when the vaccines involve new technologies that haven't been tested for safety or adapted to allow for mass production. First, a vaccine is tested in animals to see if it works and if it's safe. This testing must follow strict lab guidelines and generally takes three to six months. The manufacturing of vaccines also must follow quality and safety practices. Next comes testing in humans. Small phase I clinical trials evaluate the safety of the vaccine in humans. During phase II, the formulation and doses of the vaccine are established to prove the vaccine's effectiveness. Finally, during phase III, the safety and efficacy of a vaccine need to be demonstrated in a larger group of people.

Due to the seriousness of the COVID-19 pandemic, vaccine regulators might fast-track some of these steps. However, realistically a vaccine will take 10 to 18 months or longer to develop and test in human clinical trials. If a vaccine is approved, it will take time to produce, distribute and administer to the global population. Since people have no immunity to COVID-19, two vaccinations are needed, three to four weeks apart. People start to achieve high levels of immunity to COVID-19 one to two weeks after the second vaccination.

It is clear that the humanity needs new approaches to expedite the development and production of the options available to treat such viral infections, as drugs, antibiotics, and vaccines. Furthermore, the delivery methods for specific medication and vaccination for prevention of the viral and other types of infections need to be more effective and capable to allow an optimization of the dosage and a faster reaction of the body to their administration.

### SUMMARY OF THE INVENTION

The present invention generally includes methods and devices that produce and use focused or unfocused acoustic pressure shockwaves or specifically modulated pressure waves (that can be planar, pseudo-planar, radial, or ultrasound waves) for enhancing the productivity of the medication, genetic material, and vaccine production or for facilitating easier absorption inside the cells and thus reducing the necessary dose needed for such medications, genetic medicines, or vaccines.

The algorithm for dosage of acoustic pressure shockwaves or specifically modulated pressure waves used for production enhancement is adjusted accordingly based on specific factors that take into account the type of medication, genetic material, or vaccine needed for a specific bacterial, viral, or fungal particle. Conversely, during medication, genetic medicine, or vaccine delivery, the dosage of acoustic pressure shockwaves or specifically modulated pressure waves is fixed and non-specific to a certain type of medication, genetic material, or vaccine, since the mechanism of action is based on opening the cellular membrane for a rapid absorption of the active medication, genetic medicine, or vaccine.

In general, the focused acoustic pressure shockwaves or specifically modulated pressure waves produced by the proposed embodiments will have a compressive phase and a tensile phase during one cycle of the acoustic pressure shockwaves or pressure waves. In the compressive phase, positive compressive pressures are produced and in the tensile phase significant negative pressures are generated that create cavitation bubbles, which when reaching their full dimensions implode/collapse with high-speed jets in excess of 100 m/s. These two synergistic effects, work in tandem to stimulate cells and tissues via different mechanisms, to open temporary pores in the cellular membrane, to increase RNA or DNA production, or to call in different regeneration and growth factors, etc.

The focused acoustic pressure shockwaves consist of a dominant compressive pressure pulse, which climbs steeply up to maximum one hundred Mega-Pascals (MPa; 1 MPa=10 bar) within a few nanoseconds and then falls back to zero within a few microseconds. The final portion of the pressure profile is characterized by negative pressures of minus five to fifteen Mega-Pascals (tensile region of the acoustic pressure shockwave), with potential to generate cavitation bubbles in any kind of liquids. The cavitation bubble diameter grows as the shockwave energy is delivered to the bubble. This energy is released from the cavitation bubble during its collapse (implosion) in the form of high-speed pressure micro jets and also produce rapid transient high temperatures. However, the short duration of the shockwave pulse results in a temperature rise of <1° C., producing negligible thermal effects. For acoustic pressure shockwaves, they must be focused or concentrated (semi-focused) or completely unfocused when sent towards the point at which their effect is needed. In the targeted region in general there are two basic effects, with the first being characterized as direct generation of mechanical forces and acoustic streaming (primary effect from the positive, compressive high-pressure rise), and the second being the indirect generation of mechanical forces and acoustic microstreaming from the high velocity pressure micro jets produced by the collapse of cavitation bubbles (secondary effect from the negative, tensile pressure region). The focused shockwaves are concentrated in a focal volume that overlaps with the targeted zone, to have the maximum effects delivered by the shockwaves. If the targeted zone is placed before or after the focal volume, then the action is produced from unfocused shockwaves (before focal volume) or defocused shockwaves (after the focal volume), which are more comparable in energy, pressure signal shape, and their effects with the pressure waves (that can be planar, pseudo-planar, radial, or ultrasound waves).

Similarly, the specifically modulated pressure waves (that can be planar, pseudo-planar, radial, or ultrasound waves) have also a compressive phase and a tensile phase, which incorporate lower amounts of energy when compared to the shockwaves. Also, the pressure signal produced in the targeted zone is specific for each type of specifically modulated pressure waves (that can be planar, pseudo-planar, radial, or ultrasound waves) and ultimately dictates their action.

A focused or unfocused acoustic pressure shockwave or a specifically modulated pressure wave can travel large distances easily (based on the amount of energy put in them at the point of origination), as long as the acoustic impedance of the medium remains the same. At the point where the acoustic impedance changes, energy is released and the acoustic pressure shockwave or specifically modulated pressure waves are reflected or transmitted with attenuation. Thus, the greater the change in acoustic impedance in between different substances, the greater the release of energy is generated.

The acoustic pressure shockwaves or specifically modulated pressure waves (planar, pseudo-planar, radial, or ultrasound waves) are highly controlled to generate an energy output that will not produce any undesired damage to the tissue or cell cultures or instrumentation where they are used. This is accomplished based on energy setting (input energy level of the acoustic pressure shockwaves or pressure waves), number of acoustic pressure shockwaves or pressure waves (planar, pseudo-planar, radial, or ultrasound waves), and their frequency per second that dictates the total acoustic energy delivered in one cleaning and disinfection session. When applicable, the reflector geometry and its material directly control the delivery of the shockwaves (focused or unfocused) or specifically modulated pressure waves (pseudo-planar or radial) into the targeted region and shapes their spatial distribution. Planar waves and ultrasound pressure waves do not require a reflector. In general, the shockwaves (focused or unfocused) or pressure waves (pseudo-planar or radial) are not producing thermal effects in the targeted zone. The ultrasound waves must have low-frequency, to not produce any heating effects. Ultrasound has a compressive phase made of positive pressures and a tensile phase that encompasses the negative pressures. In comparison to the shockwaves or pressure waves, the repetition (frequency) of ultrasound phases is much higher. Thus, the ultrasound used in the embodiments presented in this invention have a frequency in between 10 to 900 kHz, and more preferable 30 to 300 kHz, which is much higher compared to 1 to 12 Hz (preferable 2 to 10 Hz) used for shockwaves or pressure waves. Also, for ultrasound the sequence of phases is continuous from positive pressure to negative pressures and then again to positive pressures in a sinusoidal continuous variation. This has an influence on the cavitation. Due to cyclical acoustic wave of the ultrasound, the cavitation bubbles growth is cyclical too and in general they do not reach the same size as the shockwave cavitation bubbles, which translates in less energy generated during their collapse.

The energy settings (energy input into acoustic pressure shockwaves or specifically modulated pressure waves as planar, pseudo-planar, radial, or ultrasound waves) directly affect the pressure output into the targeted region/zone and together with number of acoustic pressure shockwaves or pressure waves and their frequency per second, determine the total amount of energy used to produce the desired effects.

In the case of mRNA (messenger RNA) vaccines there are two categories of mRNA constructs that are being actively evaluated. The first one is the non-replicating mRNA (NRM) and second one is the self-amplifying mRNA (SAM) constructs. Non-replicating mRNA (NRM) constructs encode the coding sequence (CDS) and the self-amplifying mRNA (SAM) construct encodes additional replicase components able to direct intracellular mRNA amplification. The mRNAs are single-stranded nucleic acids transcribed from DNA, representing a critical component of gene expression. The DNA used to transcribe the mRNA, is in the form of small rings of DNA called plasmids. Each plasmid contains a virus gene, which is the genetic instruction for a human cell to build specific virus proteins or other elements and trigger an immune response to the virus. The plasmids are inserted in bacteria that is multiplied into a bioreactor for several days via nutrients and continuous movement of the nutrient broth from the bioreactor. Afterwards, the outer membrane of the bacterium is dissolved, the plasmids are released and purified from bacterial fragments. The virus gene is cut from the plasmid and linearized (make it to be straight), which gives pure DNA that is used to transcribe them into strands of mRNA. Then the non-replicating mRNA (NRM) or self-amplifying mRNA (SAM) are formulated in lipid nanoparticles (LNPs) that encapsulate the mRNA constructs to protect them from degradation and promote cellular uptake. The lipid nanoparticle is made of ionizable cationic lipids that that change their electrical charge when it enters a human cell, opening the nanoparticle and releasing the mRNA payload. Without it, a nanoparticle vaccine will not work. By mimicking the actions of endogenous mRNAs, therapeutic mRNAs avoid the immunogenic properties and manufacturing challenges associated with therapeutic recombinant proteins. mRNA expression is also temporary, and the stability of the mRNA molecule is directly linked to gene expression. The longer the mRNA says inside the cell, the greater the production of the encoded protein. The cellular uptake of the mRNA with its delivery system typically exploits membrane-derived endocytic pathways and the endosomal escape allows release of the mRNA into the cytosol (the aqueous component of the cytoplasm of a cell). The cytosol-located NRM constructs are immediately translated by ribosomes to produce the protein of interest, which undergoes subsequent post-translational modification. The SAM constructs can also be immediately translated by ribosomes to produce the replicase machinery necessary for self-amplification of the mRNA. After that the self-amplified mRNA constructs are translated by ribosomes to produce the protein of interest, which undergoes subsequent post-translational modification. The expressed proteins of interest are generated as secreted, trans-membrane, or intracellular protein and then the innate and adaptive immune responses detect the protein of interest. For vaccines, all these steps are produced in designated cell cultures and then they are followed by a filtration/separation process (both perpendicular or tangential to the filter surface). The shockwaves or specifically modulated pressure waves can be used to gentle mix the bioreactor's cellular or bacterial culture to facilitate their growth. The pure non-contact mechanical action produced by the acoustic pressure shockwaves (focused or unfocused) or specifically modulated pressure waves (planar, pseudo-planar, radial, or ultrasound waves) is manifested via acoustic streaming created by the positive pressures of the compressive phase and the acoustic microstreaming generated by the collapse of the cavitation bubbles of the tensile phase. The acoustic streaming and microstreaming can effectively and gentle mix the cellular culture or bacterial culture inside the bioreactor and avoid the strong shear forces produced by a paddle system that is currently used. In this way a gentler stirring is achieved, which eliminates unnecessary death of the cells or bacteria produced by the shear forces generated by the mechanical paddle stirrers. Interesting to note that the same shockwaves or specifically modulated pressure waves or ultrasound can also play a role in the membrane filtration processes, or in the separation processes for vaccines or vaccine components, since the shockwaves or pressure waves or ultrasound produce acoustic streaming and microstreaming that are pushing particles in preferred directions (unidirectional), which overlap with the longitudinal direction for propagation of the shockwaves or pressure waves.

Inflammation is a component intrinsic to all mRNA vaccines, given that several intracellular innate immune response sensors are activated by RNA. Similarly, other types of vaccines also produce inflammation due to the immune response, which can be more severe or not depending on the type of vaccine, the strength of immune system and comorbidities associated with each individual. Also, some medications or genetic medicines produce a local inflammation at the injection site. This is where acoustic pressure shockwaves (focused or unfocused) or specifically modulated pressure waves (planar, pseudo-planar, radial, or ultrasound waves) administration during vaccination or medication/genetic medicines delivery can help with modulation of the inflammation and thus tolerability of the vaccine, medication, drug, or genetic medicines.

Majority of the vaccines, need to be frozen or refrigerated. Work is ongoing to reliably produce vaccines that can be stored outside the cold chain, since these will be much more suitable for use in countries with limited or no refrigeration facilities. Also, the delivery of the mRNA vaccine effectively to cells is challenging, since free RNA in the body is quickly broken down. To help achieve delivery, the RNA strand is incorporated into a larger molecule to help stabilize it and/or packaged into particles or liposomes. Similar or other kinds of challenges can occur with other types of vaccines or genetic medicines or medications. In all situations, a rapid delivery inside the cell of vaccines or genetic medicines or medications is imperious and this is where the mechanotransduction produced by acoustic pressure shockwaves (focused or unfocused) or specifically modulated pressure waves (planar, pseudo-planar, radial, or ultrasound waves) on the cells is useful. Through the mechanotransduction mechanism, the shockwaves or pressure waves open effectively pores in the cellular membrane, which can help with rapid delivery inside the cells and thus being an effective vaccine or allowing local administration of drugs, medication, or genetic medicine, which allows the administration of appropriate doses and avoids possible systemic side effects.

An important challenge of vaccines is the production on the big scale. Since the mRNA vaccines are produced with the latest technology, the production challenge is even bigger. New processes and instrumentation need to be created to allow a rapid scalability and a stable and efficacious vaccine. As part of the manufacturing process the translation of in vitro transcribed mRNA transfected into cultured cells is needed. As mentioned before, this is the step where acoustic pressure shockwaves (focused or unfocused) or specifically modulated pressure waves (planar, pseudo-planar, radial, or ultrasound waves) can increase the production of mRNA due stimulation of the cultured cells to produce more mRNA or multiplication of bacteria that contain genetic material used to transcribe into mRNA, as demonstrated in scientific publications. Such increased efficiency of this production step can be stimulated due to the pure non-contact mechanical action produced by the acoustic pressure shockwaves (focused or unfocused) or specifically modulated pressure waves (planar, pseudo-planar, radial, or ultrasound waves). The gentle stirring of the nutrient broth and cells or bacteria mixed in it, combined with possible cellular or bacterial stimulation, can accomplish an increased production output.

In general, the optimization and enhancement of production process for any type of vaccines, or genetic medicines, or stem cells, or immune cells, or drugs, or medications production can be done by the use of the pure mechanical action produced by the acoustic pressure shockwaves (focused or unfocused) or specifically modulated pressure waves (planar, pseudo-planar, radial, or ultrasound waves). Bioreactor's efficiency can be increased by shockwave or pressure wave stirring and stimulation of cell or bacterial cultures or through facilitating different chemical reactions.

Currently, the introduction of the medication or genetic material inside the cells is done via a process called electroporation. Electroporation, or electro-permeabilization, is a microbiology technique in which an electrical field is applied to cells in order to increase the permeability of the cell membrane, allowing chemicals, drugs, or DNA to be introduced into the cell (also called electro-transfer). Electroporation has proven efficient for use on tissues in vivo, for in utero applications. One downside to electroporation, however, is that after the process the gene expression of over 7,000 genes can be affected. This can cause problems in studies where gene expression has to be controlled to ensure accurate and precise results. Although bulk electroporation has many benefits over physical delivery methods such as microinjections and gene guns, it still has limitations including low cell viability. Furthermore, the devices associated with the electroporation are complicated and have moving parts that makes them less reliable. This is where the acoustic pressure shockwaves (focused or unfocused) or specifically modulated pressure waves (planar, pseudo-planar, radial, or ultrasound waves) are superior to electroporation, since there is no extra-activation of genes during their action and also the shockwave/pressure wave systems do not have moving parts, which significantly increases their longevity and reliability.

The mRNA uptake and expression in vivo are dependent on hydrodynamic pressure that may contribute to target cell transfection in case of local injections, as it does upon intravenous administration. However, the correlation between pressure and transfection efficiency/protein expression may not be linear but shows an optimum. Anyway, a large amount of the mRNA appears to stay trapped in endosomal vesicles. Hence, mRNA vaccines may profit strongly from approaches increasing the fraction of mRNA that reaches the cellular cytosol. This is where the shockwaves can be used immediately after delivery of the vaccine inside the tissue to stimulate via mechanotransduction the opening of the cell's vesicles, which assures a rapid absorption of the mRNA inside the cells and the reach of the cytosol, where they produce the protein of interest/antibody. The same approach is valid for pushing any genes from gene medicines inside the cells or any liquid drug or medication, regardless of molecular dimension. Interesting to note that the pores open on cell membranes by shockwaves or pressure waves are big enough to allow large particles transport across the membrane (genetic material or large molecular substances), which is difficult to achieve in normal conditions. That creates the opportunity of local delivery of medications or drugs made of large molecules and avoid their systemic administration that usually is producing a lot of side effects. Many times, experimental medications are dropped exactly for their side-effects produced by a systemic delivery. By using the acoustic pressure shockwaves (focused or unfocused) or specifically modulated pressure waves (planar, pseudo-planar, radial, or ultrasound waves) to open large pores on the cell membrane, it will allow the coordinated delivery of medication locally (where is really needed) and thus should reduce the amount or dosage of medication needed and even reduce the local side effects.

In certain embodiments, shockwave or specifically modulated pressure waves may also be used to facilitate genetic modifications, such as in conjunction with gene therapies. Similarly, the shockwaves or specifically modulated pressure waves may be used to facilitate stem cell therapies, such as promotion and acceleration of differentiation of stem cells or prepping the targeted zone to be sure that a proper blood circulation is present (eliminate the ischemic regions). The shockwaves or specifically modulated pressure waves may be also used in combination with gene and stem cells therapies that includes the application of shockwaves or pressure waves simultaneously with gene therapy and stem cell treatment, which accelerates their intake in the cells or tissue and their additive benefits for the targeted cells or the tissue macro-structure that contains such cells. Furthermore, the acoustic pressure shockwaves (focused or unfocused) or specifically modulated pressure waves (planar, pseudo-planar, radial, or ultrasound waves) may be first applied in a combination treatment with a gene therapy that is followed by a subsequently stem cell therapy. Following the successful implantation of genes medicine and stem cells, the shockwaves or specifically modulated pressure waves may be applied to the targeted region to stimulate blood circulation and maintain the viability of the genetic material and stem cells, which assures a successful treatment of the respective tissue. Conversely, the acoustic pressure shockwaves (focused or unfocused) or specifically modulated pressure waves (planar, pseudo-planar, radial, or ultrasound waves) may be first applied in a combination treatment with a stem cell therapy that is followed subsequently by a gene therapy. Also, in this situation following the successful implantation of genes medicine and stem cells, the shockwaves or specifically modulated pressure waves may be applied to the targeted region to stimulate blood circulation and maintain the viability of the genetic material and stem cells, which assures a successful treatment of the respective tissue.

### BRIEF DESCRIPTION OF THE DRAWINGS

FIG. 1 is a schematic illustration of prior art natural immunity development against a virus.

FIG. 2 is a schematic illustration of different shapes of viruses, namely adenovirus and human immunodeficiency virus, known in the prior art.

FIG. 3 is a photographic image (from Lopez-Marin L M, et al., “Shock wave-induced permeabilization of mammalian cells”, Physics of Life Reviews, (2018), https://doiorg/10.1016/j.plrev.2018.03.001) depicting pore generation by shockwaves through mechanotransduction in a mammalian cell membrane known in the prior art.

FIG. 4 is a schematic diagram illustrating features characteristic for focused pressure shockwaves known in the prior art.

FIG. 5 is a schematic diagram illustrating features characteristic for planar pressure waves known int the prior art.

FIG. 6 is a schematic diagram illustrating features characteristic for radial pressure waves known in the prior art.

FIG. 7 is schematic diagram illustrating a classic bioreactor used to manufacture vaccines or engineered tissues known in the prior art.

FIG. 8 is a schematic diagram illustrating shockwaves and pressure waves used in manufacturing process of vaccines and during delivery of vaccines for immunization for both animal and human subjects, according to one embodiment of the present invention.

FIG. 9 is a is a schematic diagram illustrating a typical ellipsoidal geometry used for focusing shockwaves as known in the prior art.

FIG. 10 is a schematic diagram illustrating a typical semi-ellipsoidal reflector used for focused shockwave applicators/devices as known in the prior art.

FIG. 11 is a schematic diagram illustrating a full ellipsoidal reflector shockwave area and focusing relationship as known in the prior art.

FIG. 12 is a schematic diagram illustrating a bioreactor using multiple shockwave/pressure wave devices to enhance manufacturing process for vaccines, according to one embodiment of the present invention.

FIG. 13A is a schematic diagram illustrating a full reflector used for shockwaves or pressure waves optimum propagation and action that can be used as a special bioreactor to increase efficiency for vaccines manufacturing, according to one embodiment of the present invention.

FIG. 13B is a schematic diagram illustrating a special bioreactor similar to the one presented in FIG. 13A, which is used together with an automation system to increase efficiency for vaccines manufacturing, according to one embodiment of the present invention.

FIG. 14A is a schematic diagram illustrating a lateral view of a fixture for cell cultures subjected to shockwaves or pressure waves to enhance and expedite biological experimentation, according to one embodiment of the present invention.

FIG. 14B is a schematic diagram illustrating a top view of the fixture from FIG. 14A for cell cultures subjected to shockwaves or pressure waves to enhance and expedite biological experimentation, according to one embodiment of the present invention.

FIG. 14C is a schematic diagram illustrating a cross-sectional view along A-A of the fixture from FIGS. 14A and 14B with cell cultures subjected to shockwaves or pressure waves to enhance and expedite biological experimentation, according to one embodiment of the present invention.

FIG. 15 is a schematic diagram illustrating a spark-gap electrohydraulic focused shockwaves system for rapid and enhanced vaccine delivery inside the cells, according to one embodiment of the present invention.

FIG. 16 is a schematic diagram illustrating a spark-gap electrohydraulic radial pressure waves system for rapid and enhanced vaccine delivery inside the cells, according to one embodiment of the present invention.

FIG. 17 is a schematic diagram illustrating a spark-gap electrohydraulic planar pressure waves system for rapid and enhanced vaccine delivery inside the cells, according to one embodiment of the present invention.

FIG. 18 is a schematic diagram illustrating a laser electrohydraulic focused shockwaves system for rapid and enhanced vaccine delivery inside the cells, according to one embodiment of the present invention.

FIG. 19 is a schematic diagram illustrating a cylindrical coil-produced electromagnetic focused shockwaves system for rapid and enhanced vaccine delivery inside the cells, according to one embodiment of the present invention.

FIG. 20 is a schematic diagram illustrating a flat coil and lens-produced electromagnetic focused shockwaves system for cleaning and disinfection of endoscopes or tubing, according to one embodiment of the present invention.

FIG. 21 is a schematic diagram illustrating a piezo crystals/piezo ceramics-produced piezoelectric focused shockwaves system for rapid and enhanced vaccine delivery inside the cells, according to one embodiment of the present invention.

FIG. 22 is a schematic diagram illustrating a piezo fibers-produced piezoelectric focused shockwaves system for rapid and enhanced vaccine delivery inside the cells, according to one embodiment of the present invention.

FIG. 23 is a schematic diagram illustrating a piezoelectric system producing planar waves for rapid and enhanced vaccine delivery inside the cell, according to one embodiment of the present invention.

FIG. 24A is a schematic diagram illustrating features characteristic for ultrasound pressure waves known in the prior art.

FIG. 24B is a schematic diagram illustrating an ultrasonic system producing low frequency ultrasound waves, as presented in FIG. 24A, for rapid and enhanced vaccine delivery inside the cell, according to one embodiment of the present invention.

FIG. 25A is a schematic diagram illustrating a syringe with pressure waves activation system used to open pores on the cell membranes before liquid medical substance injection, according to one embodiment of the present invention.

FIG. 25B is a schematic diagram illustrating a syringe with pressure waves activation system from FIG. 25A after injection of liquid medical substance and continued pressure wave delivery to achieve a rapid and enhanced liquid medical substance delivery inside the cells or tissues or organs, according to one embodiment of the present invention.

It will be appreciated that the drawings include lead lines that are not physical structures and are merely illustrated between a reference numeral/character and a corresponding detail element.

### DETAILED DESCRIPTION OF THE INVENTION

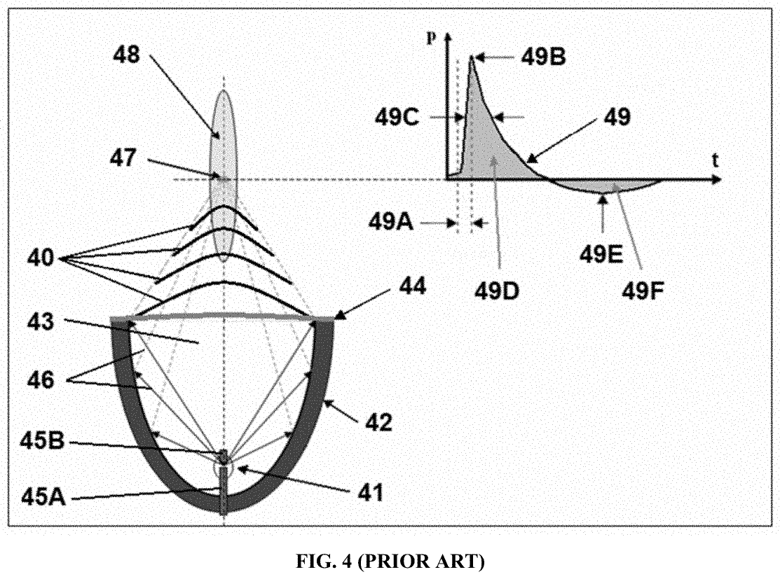

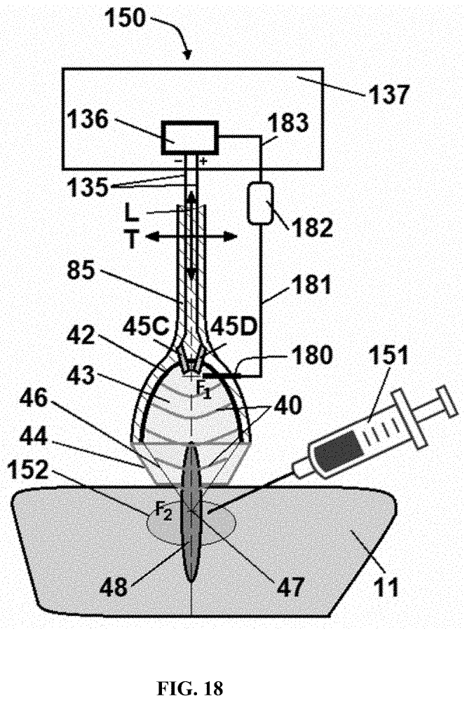

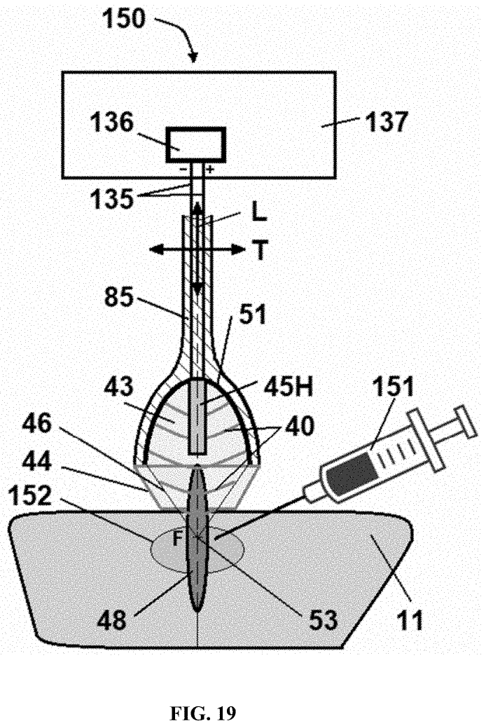

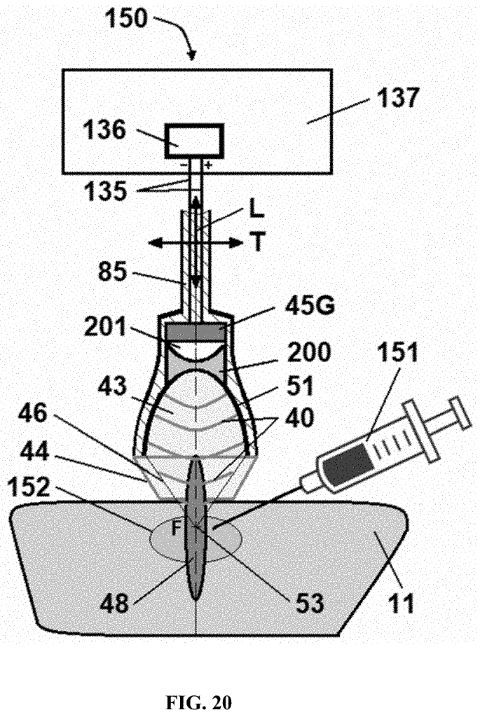

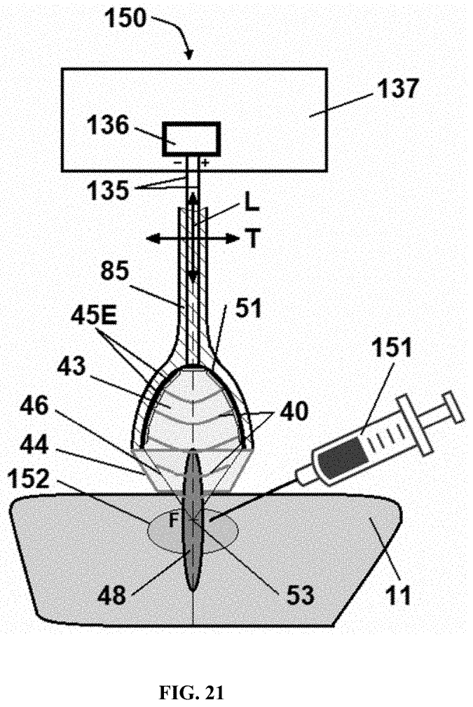

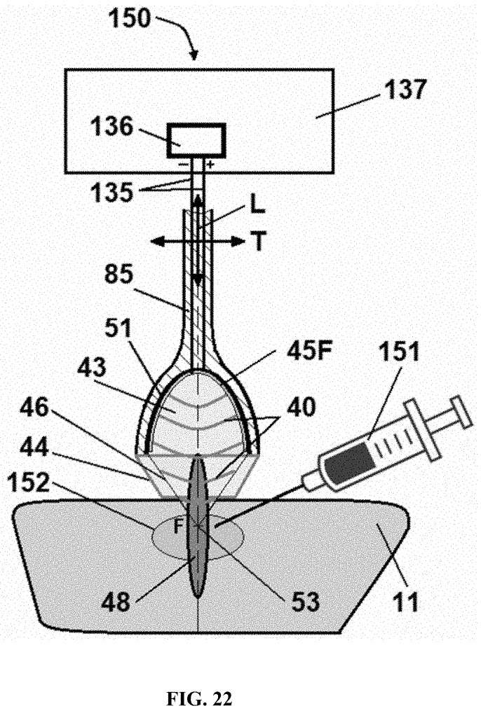

It is an objective of the present inventions to provide different methods of generating focused or unfocused shockwaves and planar, pseudo-planar, radial, or ultrasonic pressure waves using a shockwave/pressure wave generator or generators, from the following categories:
electrohydraulic generators using spark-gap high voltage discharges (see FIGS. 4-6, 10-11, 13A-13B, 14B—17)
electrohydraulic generators using one or multiple laser sources (see FIG. 18)
electromagnetic generators using a cylindrical coil (see FIG. 19)
electromagnetic generators using a flat coil and an acoustic lens (see FIG. 20)
piezoelectric generators using piezo crystals/piezo ceramics (see FIG. 21)
piezoelectric generators using piezo fibers (see FIG. 22)

For some of the figures mentioned above, although one of the principles or methods of shockwaves or pressure waves generation is specifically presented in the figure, other methods may also apply, based on each embodiment construction. That will be mentioned for each figure where such situation applies.

In general, the energy is delivered for all embodiments presented in this invention from a power supply in the form of high voltage setting for electrohydraulic and piezoelectric devices and electrical current setting for electromagnetic devices and ultrasonic devices. The power supply functionality and the parameters of the shockwave or pressure wave devices are controlled by a control console/unit, designed to have processors and microprocessors, displays, input/output elements, timers, memory units, remote control devices, independent power unit, etc. Each of these components may include hardware, software, or a combination of hardware and software configured to perform one or more functions associated with providing good functioning of the process that employs the use of the focused or non-focused acoustic pressure shockwaves or specifically modulated pressure waves (acoustic planar pressure waves or pseudo-planar pressure waves or acoustic radial pressure waves or low-frequency ultrasound waves).

Sometimes combination geometries can be used for the reflectors mentioned in the present inventions. Two or more geometries can be used as portion of an ellipsoid, combined with a portion of a sphere and a portion of a paraboloid, to give one example. That can have an effect on the way the shockwaves or pressure waves are reflected, how many focal volumes or pressure fields are created that can overlap or can be totally separated, and finally the actual focal volume or pressure field shape and its position in space.

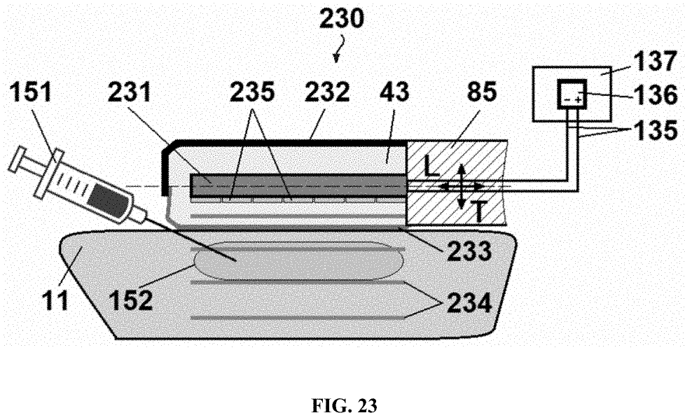

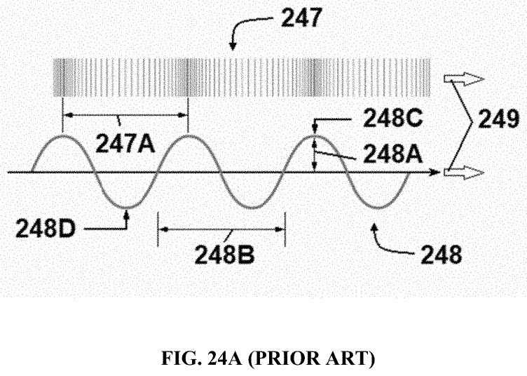

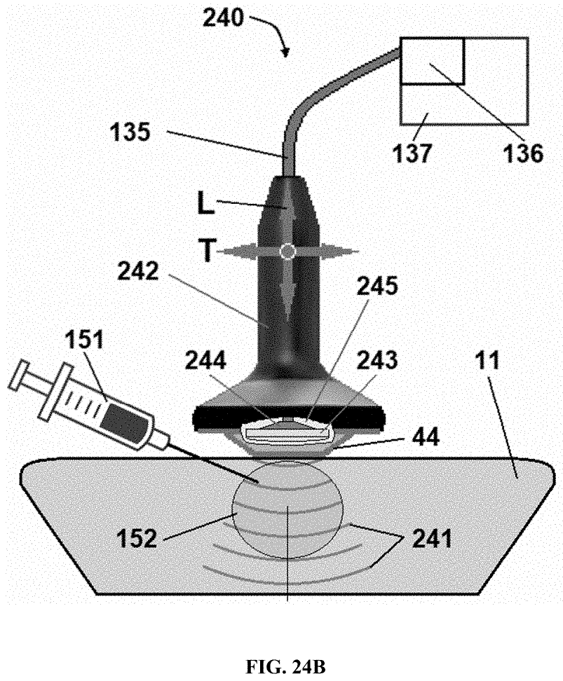

Non-rotational reflector geometries can be also used to reflect shockwaves or pressure waves. In this case, the reflector can have a pyramid geometry with triangle, square, hexagonal, or octagonal aperture. In other situations, no reflectors are used at all, where simply flat piezo-crystals (FIGS. 23, 24A, and 24B) produce a planar pressure wave or a radial pressure wave or an ultrasound wave that moves in any direction or preferred directions.

It is a further objective of the present inventions to provide a means of controlling the energy and the penetrating depth of the focused or non-focused acoustic pressure shockwaves or specifically modulated pressure waves (acoustic planar pressure waves or pseudo-planar pressure waves or acoustic radial pressure waves or low-frequency ultrasound waves), total number of shockwaves or pressure waves pulses, repetition frequency, and special construction and geometry of the reflectors and membranes used in the devices from these inventions.

For stimulation of small cell cultures or bacterial cultures in order to produce different components of the vaccines, or monoclonal antibodies (laboratory-made proteins that mimic the immune system's ability to fight off harmful pathogens such as viruses) or gene medicines, to name a few, the number of shockwaves or pressure waves preferably may be between 50 and 2000 pulses. The frequency preferably may be in between 0.5 to 2 Hz, the flux density of 0.005 to 0.100 mJ/mm², and the compressive pressures generated by the shockwaves or pressure waves preferably may vary in between 0.1 MPa to 30 MPa. The shockwaves or pressure waves are used with intermittence in this situation to allow the normal processes of the cellular or bacterial activities to take place.

For stimulation of larger cell cultures or bacterial cultures, the compressive pressures generated by the shockwaves or pressure waves preferably may vary in between 1 MPa to 50 MPa. The frequency is dependent on specific process and can be in between 0.5 Hz to 12 Hz and the flux density of 0.005 to 0.500 mJ/mm². The number of shockwaves or pressure waves can vary from 50 to 100,000 depending on the continuous or discrete regimen used for the specific small bioreactor process.

For large-scale manufacturing processes used for large bioreactors, the shockwave or pressure waves regimen it is more intensive and requires frequencies in between 2 to 12 Hz, and the flux density of 0.100 to 1.250 mJ/mm². The compressive pressures generated by the shockwaves or pressure waves preferably may vary in between 1 MPa to 100 MPa. The frequency is dependent on specific process and can be in between 0.5 Hz to 12 Hz. In this case the shockwaves or pressure waves can also be used continuously or intermittently. Careful consideration preferably may be given to not overload the cellular or bacterial batch with shockwave or pressure wave energy, which can have detrimental effects. Stimulation of cell or bacterium division and other internal processes are aimed, to allow a rapid multiplication of the desired RNA, DNA, proteins, antibodies, or viruses. This is why the number of shockwaves or pressure waves preferably may be limited for intermittent processes for each session of stimulation to 1,000 to 50,000 for sensitive processes or from 10,000 to 1,000,000 for more tolerant cellular or bacterial processes, depending on the volume of the bioreactor and the type of organism used inside the bioreactor. In general, the less energy the shockwaves or pressure waves have, the greater number of pulses are permissible to be used in a bioreactor. Besides the cellular or bacterial stimulation, the shockwaves or pressure waves or ultrasound pressure waves are also used to mix continuously or intermittently, in a gently way, the cell culture or bacterial culture and thus eliminate the classic paddle system that during stirring can produce significant shear forces, which can kill mammalian or bacterial cells.

The construction of the shockwave or pressure wave applicators/devices and associated control consoles used together as a system to stimulate vaccine intake, should have a simple construction. For most of the vaccines there preferably may be an optimum shockwave or pressure dosage (frequency, number of shockwaves or pressure waves and energy setting) that is the same and independent of the type of vaccine. The same reasoning can be applied for the delivery inside the cells of different liquid drugs, medication, genetic medicines, etc. This is why the control console do not need complicated software for the user interface in order to change many parameters (should not have too many options to change parameters). However, alternatively the control console preferably may be capable of using artificial intelligence to generate an optimum set-up algorithm for each vaccine or liquid drugs or medication or genetic medicines, etc., and thus program the treatment parameters automatically without the need of the user input. That can be accomplished by scanning a code associated with the vaccine or liquid drugs or medication or genetic medicines, etc., that will choose the right algorithm for stimulating the rapid intake after local injection of the vaccine or liquid drugs or medication or genetic medicines, etc.

The use of shockwaves or pressure waves for creating permeability of the cells to allow a quick uptake of the vaccines or liquid drugs or medication or genetic medicines, and other treatment agents, preferably may be done at frequencies in between 2 and 4 Hz, and using flux density of 0.010 to 0.300 mJ/mm². The total number of shockwaves preferably may vary in between 50 to 200 shockwave or pressure wave pulses and the compressive pressures generated by the shockwaves or pressure waves preferably may vary in between 5 MPa to 40 MPa. It will be appreciated that the application of the shockwaves or pressure waves are applied in a manner that avoids killing or destroying cells and so a majority of cells in a targeted tissue, and thereby a majority, and preferably most, if not all, of the targeted tissue, remains viable for promoting the intended treatment. In preferred embodiments, the targeted tissue to which the shockwaves or pressure waves are applied within the parameters that avoid killing and destroying cells is overlapped by a focal volume or pressure field of the shockwaves or pressure waves.

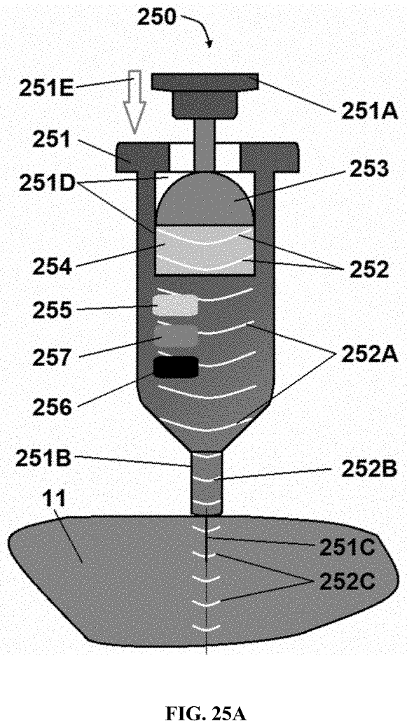

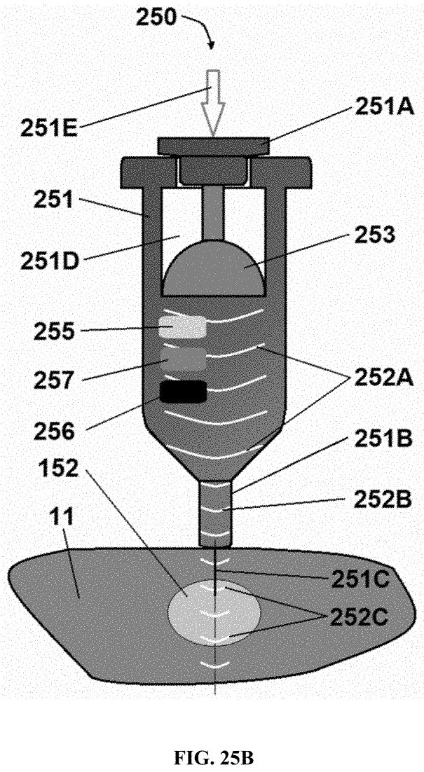

The extracorporeal shockwaves or pressure waves or ultrasound pressure waves can be applied prior or after vaccination or injection of a drug, medication, or genetic medicine, etc., depending on the main component of the vaccine or drug or medication or genetic medicine, etc. Another situation is to do the injection and the stimulation of the cells in the same time with medication intake special syringe system 250, as presented in FIGS. 25A and 25B. If the component is fragile, then the shockwaves or pressure waves or ultrasound waves preferably may be applied prior to injection, and the area preferably may be marked to be able to do immediately the subsequent injection of the vaccine, drug, medication, or genetic medicine, etc., inside the stimulated area. However, the preferred method is to apply the shockwaves or pressure waves or ultrasound waves immediately after the vaccine, drug, medication, or genetic medicine or immune cells, etc., injection, based on the fact that the precise area that needs stimulation is much clear. In this case, the healthcare professional should position the shockwave or pressure wave or ultrasound applicator/device in such way to be centered directly on top of the injection puncture, which can be easily seen. In both cases (shockwaves or pressure waves or ultrasound waves treatment prior or after injection), the shockwaves or pressure waves or ultrasound have sufficient penetration to allow both superficial and deep cell stimulation for the rapid intake of the vaccine or drug or medication or genetic medicine, etc. If the shockwaves or pressure waves or ultrasound waves stimulation is done after vaccine, drug, medication, or genetic medicine injection, then this situation has another advantage given by the presence of the liquid vaccine inside the tissue, which will enhance the produced cavitation with benefic effects on permeabilization of the mammalian cells.

Heat might be applied as an adjuvant on top of some processes that involve shockwaves or pressure waves or ultrasound to facilitate different chemical reactions and also for maintaining the cell or bacterial cultures at certain desired temperatures. Conversely, some processes may require temperatures close to refrigeration. Shockwaves or pressure waves or ultrasound can work very well at low temperatures too, due to the fact that they do generate negligible heat during their usage (few degrees Celsius after thousands of shockwaves or pressure wave pulses or after short periods of time of applying the low-frequency ultrasound).

For producing mRNA vaccines, the scale up process is accomplished in some cases enzymatically. In a series of clean rooms, enzymes repetitively transcribe DNA templates into copious strands of mRNA, which are then formulated into lipid nanoparticles. Shockwaves or pressure waves or ultrasound can be used during the phase of transfection, where live cell cultures are used to mimic natural processes, and stimulate the production of discrete elements as DNA, nude mRNA, or proteins, or even live viruses. For the mRNA vaccines, enzymes are used to pry open the DNA templates and transcribe them into strands of mRNA. If enzymatic processes are employed, then the shockwaves or pressure waves or ultrasound can enhance and expedite these processes due to continuous agitation of the culture and increase of enzymatic activity. This is done via the movement of fluid that facilitate the enzymatic reactions, which is produced by the acoustic streaming of the compressive phase of the shockwaves or pressure waves or ultrasound or due to acoustic microstreaming produced by the collapse of cavitational bubbles that were generated by the negative pressures from the tensile phase of the shockwaves or pressure waves or ultrasound.

Typically, immune response to a vaccine takes days to weeks according to many textbooks. This is where shockwaves or pressure waves or ultrasound can induce the permeabilization of mammalian cells, which allow the immediate intake of the vaccine that can significantly accelerate the reaction of the body and the production of antibodies. Due to this increased efficiency in getting inside the cells, it is possible that the dosage can be reduced for one shot. The same reduction in the amount of injection stands for drugs, antibiotics, medications, mixture/cocktail of multiple active ingredients, gene medicines, stem cells, stem cell cocktails, DNA or RNA/mRNA genetic material, genetic modified material, immune cells, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, antibodies, stem cells, and the like. The possible reduced dose, due to facilitated intake by the shockwaves or pressure waves or ultrasound, translates in more dosages produced in one manufacturing batch and also in reduction of the side effects.

In some cases, the shellfish is used to extract the allergen for the development of a vaccine. That extraction can be done via shockwaves or pressure waves or ultrasound. Even more, as discussed before the shockwaves or pressure waves or ultrasound can be used in the manufacturing process of the vaccine and also for the efficient delivery of the vaccine. The same approach can be applied to other severe food or chemical allergies. All these approaches are applicable to the processes and embodiments described in these inventions.

Tuberculosis (TB) is prevented usually via a vaccine. The shockwaves or pressure waves or ultrasound can be used in the fabrication process and on proper delivery of the vaccine. Furthermore, the shockwaves or pressure waves or ultrasound can be used as a therapy for the lungs, based on their action on lung tissue and blood vessels stimulation that can accelerate the healing of the tissue and reduce scarring. Also, the shockwaves or pressure waves or ultrasound can be used to much easily move the mucus accumulation into the lungs, which helps with clearing of the airways, as presented in the U.S. patent application Ser. No. 17/221,562. Immune system and inflammation modulation produced by shockwaves or pressure waves or ultrasound preferably may be also helpful, as demonstrated by our clinical work. All these approaches are applicable to the processes and embodiments described in these inventions.

The Human Immunodeficiency Virus (HIV) vaccine usually resides in the blood. In this case the shockwave or pressure wave or ultrasound action is on the blood where easily cavitation bubbles can be formed. Another specific approach with blood viruses is the possibility of cleaning the blood of the virus via a dialysis processes, in which shockwaves or pressure waves or ultrasound are applied ex vivo with the purpose to destroy the membrane integrity of the virus and thus destroy the actual virus. Furthermore, the shockwaves or pressure waves or ultrasound can be used in the filtration process to prevent clogging of filtration membranes and enhance the separation of different components, based on the shockwaves or pressure waves or ultrasound unidirectional action that pushes particles in preferred directions. That translates in prolonged life for the filtration membranes that increases the time period in between maintenance cycles and the total number of serviceable cycles, which can generate important savings for the manufacturing facility. As mentioned before in this invention, the shockwaves or pressure waves or ultrasound can be also used in the manufacturing process for enhanced productivity and in getting the vaccine in a larger quantity and a shorter time-frame.

The same comments related to the HIV vaccine apply to the Hepatitis vaccine, since the Hepatitis virus circulate through blood. Furthermore, in this case the virus has the tendency of hiding and the shockwaves or pressure waves or ultrasound can be used directly on liver to get the viruses out of their hiding spots and then expose them much better to the vaccine. The principle of combination of shockwaves or pressure waves or ultrasound with the vaccine delivery and the addition of using the shockwave or pressure wave or ultrasound treatment of the liver in the same time or before or after the vaccine, will enhance the probability of reaction and cure. As mentioned before in this invention, the shockwaves or pressure waves or ultrasound can be also used in the manufacturing process for enhanced productivity and in getting the vaccine in a larger quantity and a shorter time-frame. All these approaches are applicable to the processes and embodiments described in these inventions.

Human papillomavirus (HPV) vaccine targets the HPV types that most commonly cause cervical cancer and can cause some cancers of the vulva, vagina, anus, and oropharynx. The same principle of combination of shockwaves or pressure waves or ultrasound with vaccine delivery and even a shockwave treatment of the cervix, vulva, vagina, anus, and oropharynx, will enhance the probability of reaction, absorption, and cure. The processes and embodiments described in these inventions can be applied not only during vaccination but also for manufacturing, since the shockwaves or pressure waves or ultrasound can be used to enhance vaccine fabrication productivity and in getting the vaccine in a larger quantity and a shorter time-frame.

Ebola Dengue, West Nile, Zika and Chikungunya viruses also require specially designed vaccines, since they are very dangerous viruses. The processes and embodiments described in these inventions can be employed, since the shockwaves or pressure waves or ultrasound can be used during manufacturing process of these vaccines and during delivery of such vaccines, which through preparing the area of vaccination will allow a much faster integration of the vaccine into the targeted tissue or cells, with immediate enhanced response to the vaccination.

Shingle's vaccine is related to the Herpes virus. In this case, the processes and embodiments described in these inventions can be used, since the shockwaves or pressure waves or ultrasound can enhance the manufacturing processes and can be used during injection or vaccination on the specific site, for an efficient delivery of the vaccine. Furthermore, this virus has the tendency of hiding and the shockwaves or pressure waves or ultrasound can be used directly on the specific location where viruses hide, to get them out of their hiding spots and then expose them much better to the vaccine.

Similarly for the influenza vaccine against the flu viruses, the processes and embodiments described in these inventions can be used to enhance the production of such vaccine, which is necessary every flu season. The quantity of such vaccines is significant and any step that assures a much more productive manufacturing process, it is of great interest. This is where the shockwaves or pressure waves or ultrasound can play a significant role to enhance the production of larger vaccine quantities in a shorter time frame.

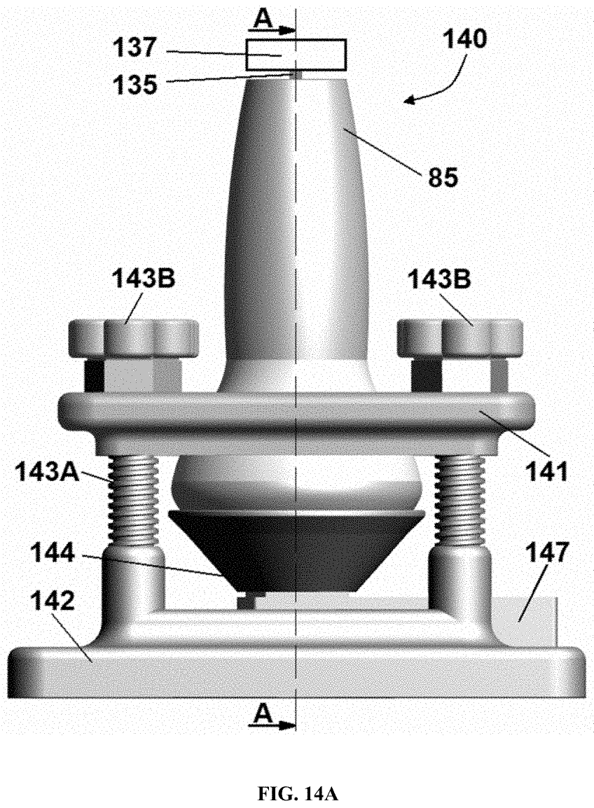

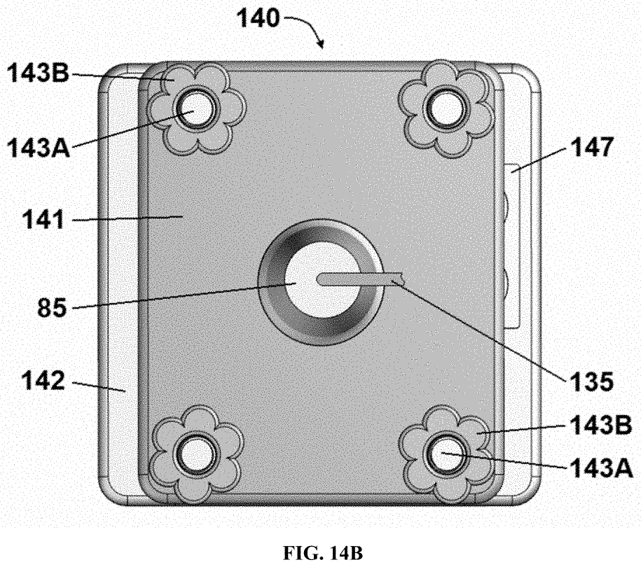

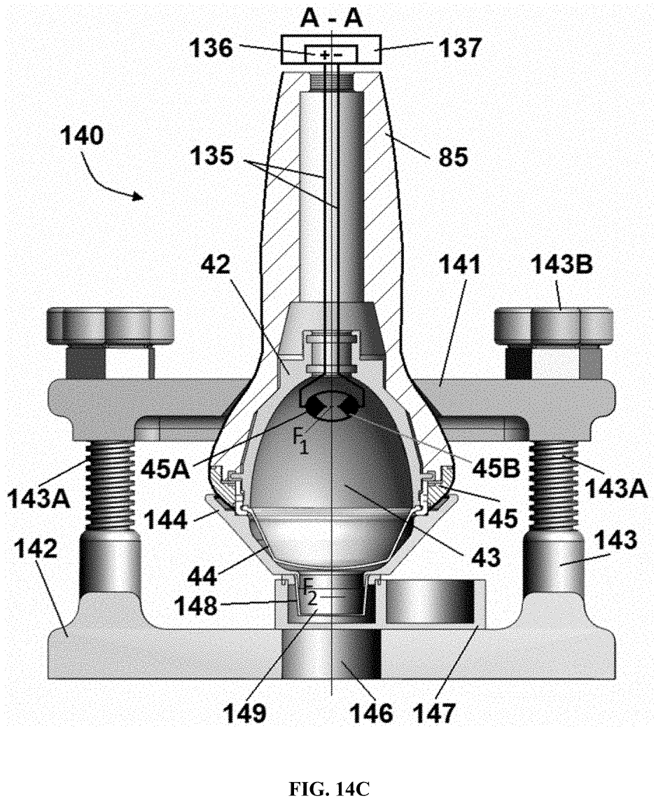

Some of the treatments against viruses are based on the antibodies taken from the plasma of patients that developed immunity after infection. Shockwaves or pressure waves or ultrasound systems or devices presented in these inventions can be used to separate the antibodies from the blood plasma and stimulate the multiplication of such antibodies by means of similar processes as described for vaccines. The gentle mechanical stimulation produced by the shockwaves or pressure waves or ultrasound in the presence or absence of viral components, can produce increased number of antibodies, thus reducing the necessity for periodic blood donations from such patients with immunity. The enhanced processes described here in these inventions are lab based and can employ embodiments as the one presented in FIGS. 14A and 14B.

The manufacturing processes for some medication require laborious steps of extraction and multiplication of different key components form plants, animals, etc. Due to the pure mechanical and non-direct contact action of the shockwaves or pressure waves or ultrasound, they can assure a complete non-contamination of the batch, can enhance chemical reactions, or mix immiscible fluids that might be crucial for certain reactions. In this way the shockwave or pressure wave or ultrasound systems can eliminate the need of use of expensive mixing equipment that also might pose the risk of contamination due to their specific construction or can produce delirious effects on the cells or molecules used in the manufacturing process. Important to note is that the shockwave or pressure waves or ultrasound systems do not have moving parts, which increase exponentially their reliability and maintenance simplicity.

Shockwaves or pressure waves or ultrasound can be used to enhance the drug delivery from skin patches or subcutaneously patches, by selectively activating a certain drug and mechanism of action, based on the associated shockwave or pressure wave or ultrasound energy used for such specific activation or delivery.

The big drawback for many drugs, medications, gene medicines, immune cells, antibodies, or stem cells therapeutics is represented by the adverse effects generated when the dosages are enhanced to provide a better therapeutically effect. Many drugs, medications, gene medicines, or stem cells therapeutics that must be delivered systemically, via gastro-intestinal or blood circulatory routes, are dropped out during research phase due to their delirious side effect at the effective dosages. The shockwaves or pressure waves or ultrasound can play an important role for such drugs, medications, gene medicines, immune cells, antibodies, or stem cells therapeutics. If such drugs, medications, gene medicines, immune cells, antibodies, or stem cells therapeutics (generically called “treatment elements”) are encapsulated in liposomes, lipid nano or micro-particles, or any other artificially or natural envelope, they can be delivered to the specific tissue via a systemically approach without adverse effects. When these treatment elements that are encapsulated in special envelopes reach the desired tissue, shockwaves or pressure waves or ultrasound can be applied extracorporeally to the specific tissue or organ, and break these envelopes to deliver safely a high dosage of the respective active treatment element where is needed, without side effects in other parts of the body. The size of the envelopes/microparticles used will dictate the type of tissue that is targeted (matching the interstitial or intracellular spaces of that specific tissue), where such enveloped-treatment elements should concentrate. The intact envelopes that did not reached the desired tissue will be safely eliminated via urinary or gastric tracts. The extracorporeal approach and the possibility to penetrate deep inside the human and animal body, makes the shockwaves or pressure waves or ultrasound the ideal delivery system for such enveloped drugs, medications, gene medicines, immune cells, antibodies, or stem cells therapeutics.

Sometimes the delivery of a certain active substance or genetic material or cellular material in the desired tissue or organ is hindered by the excessive inflammation and/or scar tissue (no adequate blood circulation). The shockwaves or pressure waves or ultrasound are known to increase blood circulation via blood vessels dilatation, to modulate inflammation, to create new blood vessels and tissue, and to reduce or eliminate of scarring, which makes the shockwaves or specifically modulated pressure waves or low-frequency ultrasound ideal to prepare the treatment targeted region before the drug or medication or gene medicines or stem cells delivery. This will enhance the uptake of the drug, medication, antibiotics, gene medicine, protein material, or cellular material (stem cells, immune cells, etc.) after its delivery, by using locally shockwaves or pressure waves or ultrasound tissue activation. Furthermore, the shockwaves or pressure waves or ultrasound can change ischemic areas in non-ischemic ones (due to grow of new small blood vessels and new tissue regeneration), which also helps with the success of the treatment.

In certain embodiments of these inventions, the shockwaves or pressure waves or ultrasound may also be provided to enable genetic modifications, such as in conjunction with gene therapies, and also to facilitate stem cell therapies, such as promotion and acceleration of differentiation of stem cells, and thus helping with a combination of gene and stem cells therapies. These shockwaves or pressure waves or ultrasound facilitations of gene therapies and/or stem cells therapies can enhance the absorption of genetic material inside the cells or produce gene modification of the cells or expedite the action of the stem cell treatment. This effect produced by the shockwaves or pressure waves or ultrasound can reduce the number of genetic material or stem cells needed for one dosage, with significant implication in reducing possible adverse reactions at the recipient, which can enhance the adoption of such treatments that are now plagued with numerous adverse events. Also, from the manufacturing point of view, if one injection or dosage requires less genetic load (DNA, RNA, mRNA, gRNA—guide RNA) or less stem cells or immune cells, this will allow the production of much more dosages with the same amount of genetic or stem cell material that was produced in one batch from one bioreactor.

The first generation of gene editing was CRISPR (acronym for clustered regularly interspaced short palindromic repeats), a technology developed in 2012 that can target and cut sections of DNA like a pair of scissors. CRISPR gene editing is a genetic engineering technique in molecular biology by which the genomes of living organisms may be modified. It is based on a simplified version of the bacterial CRISPR-Cas9 (CRISPR associated protein 9) antiviral defense system. By delivering the Cas9 nuclease complexed with a synthetic guide RNA (gRNA) into a cell, the cell's genome can be cut at a desired location, allowing existing genes to be removed and/or new ones added in vivo. Another technique for gene editing is known as “Base Editing”, which works more like a pencil that can target a single misspelling in the DNA code allowing for much greater precision. These technologies could theoretically cure thousands of the genetic diseases caused by single letter misspellings, known as point mutations. That opened the possibility of creating the gene medicines, which are gaining traction in treating numerous diseases.

Examples of rare genetic disease that might benefit from CRISPR or Base Editing technologies and associated genetic treatments are presented below.

Sickle Cell Disease, which is an inherited blood disorder causes severe pain. Abnormal hemoglobin molecules—hemoglobin S—stick to one another and form long, rod-like structures. These structures cause red blood cells to become stiff, assuming a sickle shape. Their shape causes these red blood cells to pile up in small vessels, producing blockages and damaging vital organs and tissue.

T-Cell Acute Lymphoblastic Leukemia, which can produce fast-growing blood cancer. Acute lymphocytic leukemia (ALL) is also called acute lymphoblastic leukemia. “Acute” means that the leukemia can progress quickly, and if not treated, would probably be fatal within a few months. “Lymphocytic” means it develops from early (immature) forms of lymphocytes, a type of white blood cell.

Acute Myeloid Leukemia is another disorder that produces fast-growing blood cancer. Acute myeloid leukemia (AML) is a cancer of the myeloid line of blood cells, characterized by the rapid growth of abnormal cells that build up in the bone marrow.

Alpha-1 antitrypsin (AAT) deficiency is an under-recognized hereditary disorder associated with the premature onset of chronic obstructive pulmonary disease, liver cirrhosis in children and adults, and less frequently, relapsing panniculitis, systemic vasculitis and other inflammatory, autoimmune and neoplastic.

Glycogen Storage Disorder 1a Inherited disorder where body can't store sugar. Glycogen storage disease type 1 is an inherited disorder caused by the buildup of a complex sugar called glycogen in the body's cells. The accumulation of glycogen in certain organs and tissues, especially the liver, kidneys, and small intestines, impairs their ability to function normally.

Stargardt disease is also called Stargardt macular dystrophy, juvenile macular degeneration, or fundus flavimaculatus. The disease causes progressive damage or degeneration of the macula, which is a small area in the center of the retina that is responsible for sharp, straight-ahead vision.

Angelman syndrome is also a genetic condition. Most people with Angelman syndrome have a gene called UBE3A that is absent or faulty. When this gene is faulty or missing, nerve cells in the brain are unable to work properly, causing a range of physical and intellectual problems.

Ankylosing spondylitis is a kind of arthritis that affects the joints and ligaments of your spine. Ankylosing’ means stiff and spondylo means vertebra. Ankylosing spondylitis can affect other large joints, and can be related to problems in eyes, skin, bowel and heart.

Apert syndrome is a rare genetic condition, usually evident at birth, that causes an abnormally shaped skull and fused fingers and toes. Other body parts and organs are also affected.

Charcot-Marie-Tooth (CMT) genetic disease is an inherited neurological condition that causes problems with the muscles of the feet, legs, arms and hands.

Cystic fibrosis is a genetic disease that mostly affects the lungs and digestive system. It results from a fault in a particular gene. As a result, the mucus produced by the lungs and intestines to be thick and sticky.

Duchenne muscular dystrophy (DMD) is a severe type of muscular dystrophy that primarily affects boys. Muscle weakness usually begins around the age of four, and worsens quickly. Muscle loss typically occurs first in the thighs and pelvis followed by the arms. This can result in trouble standing up. Most are unable to walk by the age of 12. Affected muscles may look larger due to increased fat content. Scoliosis is also common symptom. Some patients may have intellectual disability. Females with a single copy of the defective gene may show mild symptoms.

Haemochromatosis is an inherited genetic condition that causes the body to absorb too much iron. In some cases of haemochromatosis, the extra iron can lead to organ damage.

Haemophilia is a bleeding disorder caused by a gene mutation. Due to haemophilia, the blood does not clot properly, which makes it difficult to control bleeding. When a blood vessel is injured, special proteins in the blood called ‘clotting factors’ act to control blood loss by plugging or patching up the injury. People with haemophilia have lower than normal levels of a clotting factor.

Huntington's disease is an inherited genetic condition that affects the nervous system. Although Huntington's disease can occur at any age, symptoms often don't appear until middle age. Main symptoms are stiffness, involuntary movements, changes in balance and coordination, loss of control of bodily functions such as swallowing and speaking, fatigue, difficulty concentrating, and deterioration of memory, judgement and speed of thought.

Klinefelter syndrome is a genetic condition affecting males. It occurs if a man is born with an extra X chromosome. It can cause a variety of problems, including a small penis, small testes, and infertility.

Marfan syndrome is caused by a gene abnormality, specifically a change (mutation) in the gene that affects the elasticity of tissues that holds together muscles and joints.

Neurofibromatosis is a genetic condition characterized by the growth of benign tumors. The symptoms include light brown spots on the skin, freckles in the armpit and groin, small bumps within nerves, and scoliosis. Also, there may be hearing loss, cataracts at a young age, balance problems, flesh colored skin flaps, and muscle wasting.

Prader-Willi syndrome is a rare genetic disorder that causes a range of physical, intellectual and behavioral problems.

Rett syndrome is a genetic condition that affects the nervous system, causing intellectual and physical disability. Rett syndrome is a rare genetic disorder caused by a mutation in a gene on the X chromosome. It is named after Andreas Rett, the doctor who originally described it. The disorder usually results from a random genetic mutation rather than being inherited. It mainly affects girls.

Tay-Sachs disease also known as Progeria is a genetic disorder that leads to the premature death of young children. Babies with infantile Tay-Sachs disease appear healthy at birth. But by the time they are 6 months old, their development is slowing. They gradually lose power and movement in their limbs, and lose their vision. Over time the children regress in other ways, losing the power of speech and many other functions. Most children with infantile Tay-Sachs disease die before getting to school age.

Thalassaemia is an inherited genetic disorder that affects the blood. People with thalassaemia do not produce enough healthy haemoglobin. Inherited blood disorder causes severe anemia.

Von Willebrand genetic disease is an inherited bleeding disorder. People with von Willebrand disease have problems controlling their bleeding.

One of the big technical challenges for the treatment of many of the above-mentioned genetic diseases, where gene medicines are created via CRISPR and Base Editing, is the refining of the delivery methods that assure the transfer of modified genetic material into the patient's body. While for some of modified genetic material treatments, the actual genetic modification that creates the gene medicines can be done outside the body and then inserted into the body, for other diseases, like Progeria, the base editor will have to be directly inserted into the patient. The challenge is the creation of a delivery system that is going to take the genetic material or a Base Editing apparatus and efficiently and safely get it to the cells where it needs to do their work. For that, the cells need to open membrane pores and allow the selectively delivery inside them of the genetic material, via viruses or other vectors, without damaging the mammalian cells.

The shockwaves or pressure waves or ultrasound can open pores in the membranes of the mammalian cells through mechanotransduction mechanism, which is produced by rapid variation in pressures generated by shockwaves or pressure waves or ultrasound from high compressive pressures to negative pressures in the targeted treatment region. In embodiments of the invention, shockwaves or pressure waves or ultrasound can be applied to stimulate the targeted tissue or cells via mechanotransduction, and produce the opening of the cell's membrane vesicles without destroying the cell, which assures a rapid absorption into the cells of the subsequently injected genetic material or genetic medicines, via viruses and various vectors used during gene therapy. The shockwaves or pressure waves or ultrasound facilitating such absorption as part of the gene therapy allows an enhanced targeted delivery of the genetic treatment (normal genes or genetic modified material or the base editing means/apparatus) inside the targeted tissue or cells. Furthermore, the shockwaves or pressure waves or ultrasound can be used after injection/delivery to enhance body absorption due to formation of new blood vessels and enhanced growth factors. Additionally, shockwaves or pressure waves or ultrasound can help reduce the dosages of gene medicines for gene therapies needed for a treatment, since the facilitation of rapid absorption should allow a reduction in the dosage of vectors and genetic material needed to produce the desired genetic modification. The reduction in genetic material required for one gene therapy session, can significantly reduce the side effects, which sometimes can kill such treatments.

Furthermore, sometimes the delivery of a certain active substance (vaccines, drugs, antibiotics, medications, mixture/cocktail of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like) in the desired tissue or organ is hindered by the excessive inflammation and/or scar tissue and no adequate blood circulation. Shockwaves or pressure waves or ultrasound are known to increase blood circulation via blood vessels dilatation, to modulate inflammation, and to reduce or eliminate of scarring, which in some embodiments allows shockwaves or pressure waves or ultrasound to be used to prep the treatment area before the specific treatment. This will enhance the uptake of the vaccines, drugs, antibiotics, medications, mixture/cocktail of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, antibodies, base editing means, stem cells, and the like. In certain embodiments, before/during/after injection or oral administration or local delivery of active substance or ingredients or materials/nano-materials or stem cells or immune cells or genetic materials or mixture/cocktail of multiple active ingredients, the shockwaves or pressure waves or ultrasound can be applied to promote the precise delivery inside tissue of the treating substances and facilitate proper activation of the treatment in desired/targeted areas of the body.

Moreover, the shockwave or pressure waves or ultrasound treatment can be periodically applied, after initial injection or oral administration or local delivery of the treatment of active substance or ingredients or materials/nano-materials or stem cells or immune cells or genetic materials or mixture/cocktail of multiple active ingredients, to continue to improve blood circulation and produce tissue or cellular activation and stimulation, to be sure that the treatment is effective and properly sustained by enough oxygenation, nutrients, and the like factors. In the case of the stem cells, the fast differentiation of the stem cells in the type of cells needed for repair can be promoted due to shockwaves or pressure waves or ultrasound action. This is even more efficient when stem cell encapsulation technology (stem cells in a capsule) is used to improve quality and reduce waste. Encapsulated stem cells spontaneously self-organize in an in vivo-like 3D conformation promoting fast and homogeneous growth, as well as genomic stability. The resulting 3D stem cell colony can then be subjected to shockwave or pressure waves or ultrasound directly through the capsule, to promote their differentiation into functional microtissues ready for transplantation.

By using the shockwaves or pressure waves or ultrasound as adjunct system for proper delivery and reaching the desired penetration and finally the activation of the active substance or ingredients or materials/nano-materials or stem cells or immune cells or genetic materials or mixture/cocktail of multiple active ingredients, there is a distinct possibility to significantly reduce the quantity of active substance or ingredients or materials or nano-materials or stem cells or immune cells or genetic materials or mixture/cocktail of multiple active ingredients needed for successful outcome. Such reduced dosages will have a significant impact on the manufacturing process for these treatments, which will allow more doses to be available for the same quantity produced and also on diminished toxicity and side effects associated with it. Furthermore, by having a lower dosage of the active substance or drug or ingredients or materials or nano-materials or stem cells or immune cells or genetic materials or mixture/cocktail of multiple active ingredients for a certain treatment, will reduce the possibility of generating side effects and rejection, which will make the treatment more tolerable and successful. Even more, drugs or medications or vaccines or gene medicines that were rejected for side effects may be brought back into the game due to the enhanced and targeted delivery produced by the shockwaves or pressure waves or ultrasound.

Lately, it was studied the introduction of anti-CRISPRs small proteins, typically 50-150 amino acids in size, into the CRISPR process. There are more than 50 such proteins known at this time, and they bear little resemblance to one another in terms of sequence or structure, suggesting they all evolved independently. Some suppress the Cas enzyme's ability to bind to DNA, whereas others prevent the system from cleaving DNA or interfere with the guide RNAs it relies on. Anti-CRISPRs are drawing the most attention for regulating CRISPR therapies that aim to remove problem genes, since one very commonly invoked concern about CRISPR genome editing is accuracy. Cas9 can target the wanted site, but the concern is that it may also end up mutagenizing other sites that are not targeted. The idea is that after delivering Cas9 and editing the desired site on the genome, an anti-CRISPR protein can be used to shut down the enzyme and suppress accumulation of off-target edits.

Furthermore, the anti-CRISPRs may also help limit gene editing to desired tissues within the body. The anti-CRISPR proteins can be used to curtail their activity when they are exposed to molecules that exist only in certain tissues, for example in liver and heart cells. In this way, a decision can be made on which cell's genome to edit. A combination of an anti-CRISPR protein with an energy-sensitive molecule can be also used as a way to switch the protein on and off. This approach gives the physician a very precise spatial and temporal control of CRISPR gene editing from outside the body. The controlling of anti-CRISPR proteins can be done with small-molecule drugs or by modifying Cas enzymes, which can be activated by using shockwaves or pressure waves or ultrasound or light or other energy means. Thus, the shockwaves or pressure waves or ultrasound can be used to control the CRISPR therapies and prevent unnecessary side effects by activating anti-CRISPR selective proteins, which will make the CRISPR treatment more efficient and well targeted for curing a certain genetic disease. Since the shockwaves or pressure waves or ultrasound are non-specific to a type of cell or targeted tissue in their temporal or spatial action, makes them a universal approach to the enhancement of the CRISPR process. Furthermore, the introduction site or type of tissue can be made more permissible to the genetic material produced via CRISPR, when shockwaves or pressure waves or ultrasound stimulation are used before, during or after genetic treatment. After successful implantation, the site can be also periodically treated with shockwaves or pressure waves or ultrasound to create new blood vessels, or grow healthy cells and tissues, and thus facilitating a successful treatment.

In different embodiments of these inventions, stem cells may also be treated with shockwaves or pressure waves or ultrasound to facilitate medical treatment at a targeted location of the body. It will also be appreciated that in some embodiments, shockwaves or pressure waves or ultrasound may be applied ex vivo in combination with stem cell therapy, gene therapies, other active substances, and the like, to generate a desired type of cell, tissue, organ or similar body elements that are subsequently introduced into the body or transplanted for in vivo treatment. Similar to stimulating cell membrane with shockwaves or pressure waves or ultrasound, to facilitate absorption of genetic material and other active substances, in the case of the stem cells after their introduction into a targeted tissue, the application of shockwaves or pressure wave or ultrasound in sufficient dosage can be used to promote differentiation of the implanted stem cells into desired cells, tissues, or organs, as targeted by the medical treatments. It is believed that shockwaves or pressure waves or ultrasound help to “open” stem cells and neighboring cells' walls/membranes and also stimulate inter-cellular communication to facilitate the proper stem cell differentiation into the right type of cells and tissues that are needed for the cure or treatment. Due to this action, the shockwaves or pressure waves or ultrasound addition thereby allows for a reduction of the number of stem cells needed to be introduced/delivered inside the targeted zone, if they are targeted and facilitated in differentiation by shockwaves or pressure waves or ultrasound. Furthermore, by applying shockwaves or pressure waves or ultrasound to tissues or cells or organs, an enhanced blood supply is also immediately produced, due to blood vessels dilation and in the long run due to new small blood vessels formation (angiogenesis). This means that when the shockwaves or pressure waves or ultrasound are applied in conjunction with the stem cell therapy, it will produce an accelerated differentiation and may also enhance the surviving of the newly differentiated stem cells in the body, due the proper nutrients being brought to the area by an enhanced blood circulation, which promotes the healing and the treatment benefic results. Due to this enhanced survivability produced by applying simultaneously or subsequently the shockwave or pressure wave or ultrasound therapy when stem cells are inserted inside the treatment region/body, a smaller number of stem cells are needed for each dosage, with the advantages mentioned before (less adverse events and less stem cells used for each treatment). This allows the reduction of the manufacturing cycle for each dose and maximizes the number of dosages per each batch of stem cells that were stimulated and multiplied in a bioreactor. Based on the same rationale, as presented in this paragraph, the same action principles and benefic effects can be used for delivering reduce dosages of active substances or drugs or ingredients or materials or nano-materials or genetic materials or mixture/cocktail of multiple active ingredients, for various treatments of human or animal or plant cells/tissues/organs.

In the embodiments of these inventions, the gene therapies and/or other active substance therapies may be utilized in combination with stem cell therapy, when the simultaneous or subsequent application of shockwaves or pressure waves or ultrasound is used. Some examples include:
introducing gene therapy to a treatment site in conjunction with application of shockwaves or pressure waves or ultrasound to facilitate the gene therapy and subsequently introducing stem cells, preferably in combination with applying shockwaves or pressure waves or ultrasound to accelerate differentiation of the stem cells into cells that mimic cells that have been “repaired” by gene therapy;
simultaneously introducing gene therapy and stem cell therapy to a treatment site in conjunction with application of shockwaves or pressure waves or ultrasound to facilitate both genetic modification and stem cell differentiation in the targeted tissue, as an enhanced treatment modality;
introducing stem cell therapy in tissues or organs in conjunction with application of shockwaves or pressure waves or ultrasound to facilitate stem cell differentiation in “healthy” cells necessary to create a proper environment for the surviving of the gene modified cells introduced subsequently via gene therapy, preferably in combination with applying shockwaves or pressure waves or ultrasound, to accelerate absorption of the genetic material used to modify cells, as desired for the treatment. It is preferable to continue periodically (at least two times per week) the shockwave or pressure waves or ultrasound treatment post-implantation of the stem cells and genetic material, to increase new blood vessels creation (neo-vascularization into the targeted region), which gives enhanced oxygenation and nutrients that assures proper integration of the new stem cells and genetically modified cells inside the tissue or organ and ultimately produces functionality regeneration of the tissue or organ.

In conclusion, it will be appreciated that drugs, nanobots/nano-robots, nanoparticles, and other active substances could be used in conjunction with gene and/or stem cell therapies and together with shockwaves or pressure waves or ultrasound to facilitate desired medical treatments. Any of the embodiments presented in these inventions can be used to accomplish these treatments.

Similarly, the genetic material or genetic modified material or the base editing apparatus can be used in conjunction with shockwaves or pressure waves or ultrasound for plants and animals. The genetic material, as DNA, RNA, mRNA, gRNA, etc., can be used for dealing with diseases in animals, or for modifying plants to increase yield and make them more resistant to diseases and parasites. This can create sustainable solutions to address some of the biggest issues facing our planet today, from public health crises to environmentally-friendly food production for a growing population. Also, such agricultural products will help farmers create greener, cleaner crops by precisely targeting a specific pest with non-toxic bio-controls, and without harming beneficial insects or leaving residues in the soil or water. The embodiments presented in these inventions can be used to accomplish these results for plants or animals.

Various applications for the embodiments of the inventions have been described above. It will, however, be evident that various modifications and changes may be made thereto, and additional embodiments may be implemented, without departing from the broader scope of the inventions as set forth by the claims. This specification is to be regarded in an illustrative rather than a restrictive sense.

Embodiments of the invention will be described with reference to the accompanying figures, wherein like numbers represent like elements throughout. Further, it is to be understood that the phraseology and terminology used herein is for the purpose of description and should not be regarded as limiting. The use of “including”, “comprising”, or “having” and variations thereof herein is meant to encompass the items listed thereafter and equivalents thereof, as well as additional items. The terms “connected”, and “coupled” are used broadly and encompass both direct and indirect mounting, connecting, and coupling. Further, “connected” and “coupled” are not restricted to physical or mechanical connections or couplings.

It is a further objective of the present inventions to provide a means of controlling the delivered energy in the treatment targeted region, via the amount of energy generated by the focused acoustic pressure shockwave, or specifically modulated pressure waves (planar, pseudo-planar, radial, or unfocused waves), or low-frequency ultrasound generators (using the energy settings), total number of the shockwaves or pressure waves, repetition frequency for the shockwaves or pressure waves or ultrasound, duration of the treatment or application, and through the special construction of the reflectors used in some of the applicators presented in the present inventions.

It is a further objective of the present inventions to provide focused shockwaves or specifically modulated pressure waves (planar, pseudo-planar, radial, or unfocused waves) or low-frequency ultrasound generating devices that are modular, do not need high maintenance and can, if needed, be applied/used in conjunction and synergy with other devices or systems.

The inventions summarized below and defined by the enumerated claims are better understood by referring to the following detailed description, which is preferably read in conjunction with the accompanying drawing/figure. The detailed description of a particular embodiment, is set out to enable one to practice the invention, it is not intended to limit the enumerated claims, but to serve as a particular example thereof.

The inventions described herein are not intended to be limited to specific embodiments that are provided by way of example, but extend to the full scope of such claims of a corresponding issued patent.

Also, the list of embodiments presented in this patent is not an exhaustive one and for those skilled in the art, new applications and optimization methods can be found within the scope of the inventions.

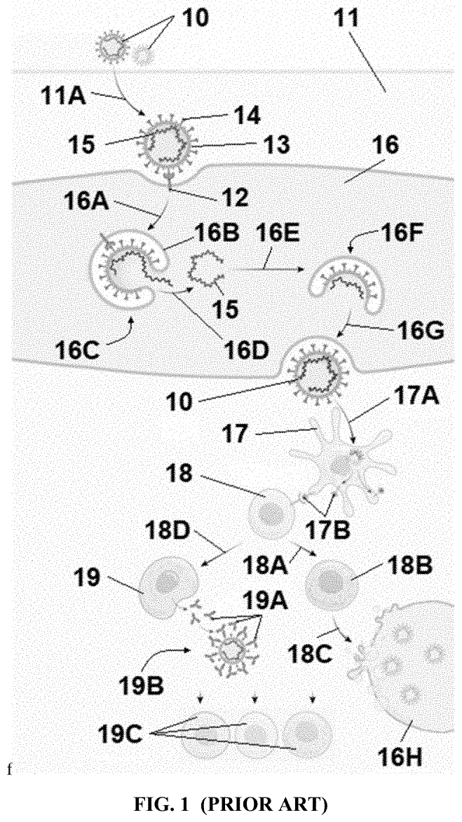

In FIG. 1 is presented the action of viruses and how an immunity is developed via vaccines against a certain type of viruses, as Corona virus 10. Immunity is developed by teaching the s immune system of the human body 11 to recognize new invading pathogens. When a Corona virus 10 is entering the body 11A, which is the first step in developing immunity process. Once in the human body 11, the Corona virus 10 latches onto ACE2 receptors 12 on the surface of human cells 16. The Angiotensin-converting enzyme 2 (ACE2) is an enzyme attached to the membrane of human cells 16. ACE2 lowers blood pressure by catalyzing the hydrolysis of angiotensin II into angiotensin. ACE2 is an enzyme that generates small proteins, by cutting up the larger protein angiotensinogen, which then go on to regulate functions in the cell located in the intestines, kidney, testis, gallbladder, heart, etc. Notably, very limited expression was seen within the respiratory system, both at protein and mRNA (messenger ribonucleic acid) level, with absent or low expression within a subset of cells or individuals. The expression within the respiratory system was mainly restricted to ciliated cells of upper airway epithelia. Corona virus 10 particles consist of a helical nucleocapsid structure, formed by the association between nucleocapsid (N) phosphoproteins and the viral genomic RNA (ribonucleic acid) 15, which is surrounded by a lipid bilayer where three types of structural proteins are inserted: the spike (S) protein 14, the membrane (M) protein 13, and the envelope (E) proteins (not specifically shown in FIG. 1 for simplicity). The membrane (M) protein 13 is a type III transmembrane glycoprotein and it is located among the spike (S) proteins 14 in the virus envelope along with small amounts of envelope (E) proteins. The membrane (M) protein 13 is the most abundant structural protein and defines the shape of the viral envelope and is the primary driver of the virus budding process.

The first step in viral infection and developing immunity process is the entering the body 11A via the air ways, when Corona viruses 10 attach to the ACE2 receptor 12 using the spike (S) protein 14. Through the ACE2 receptors 12, the human cell 16 allows the second step in developing immunity process, which is entering the cell 16A. Once virus is inside the cell 16A, the Corona virus 10 is capable to perform the third step, which is fusing with vesicle 16C. The fusing allows the fourth step in developing immunity process, which is the releasing the viral RNA 16D. The human cells 16 that are contaminated, then produce the fifth step, which is translating viral RNA in proteins 16E, to produce the new virus assembly 16F. The newly produced viruses 10 are ready to exit the human cell 16 through the sixth step in developing immunity process, which is releasing the virus from human cell 16G. At this point in time, the immune system is mounting a response to the many invading viral particles. Thus, the specialized antigen-presenting cell (APC) 17 once they find a Corona virus 10, they engulf it and produce the seventh step in developing immunity process, which is the ingesting the virus by APC 17A. After that, the antigen-presenting cell (APC) 17 are capable to display portions/fragments of the Corona virus 10, as the viral peptide displayed by APC 17B, which is attracting the T-helper cell 18. This is producing the eighth step in developing immunity process, which is the activating the T-helper cells 18A. Through this step the T-helper cells 18 transform themselves in cytotoxic T cell 18B that are capable to produce the ninth step in developing immunity process, which destroys infected cell 18C by bursting infected human cell 1611. Also, the T-helper cell 18 enable other immune response by producing the tenth step in developing the immunity process, which is T-helper cell calling the B cells 18D. The B cells 19 make antibodies 19A that can block the virus from infecting cells as well as mark the virus for destruction. This is the eleventh step in developing the immunity process, which is preventing binding or tagging virus for destruction 19B. Finally, the long-lived memory B and T cells 19C that are able to recognize the Corona virus 10 are patrolling the human body 11 for months or years, providing long-lasting immunity. The development of immunity process presented here is also valid for other types of viruses. By tapping into this process at different steps, the vaccine and drug developers can produce potent vaccines or medication that enhance the normal immune response and thus preventing the viral infection and the delirious consequences to human health.

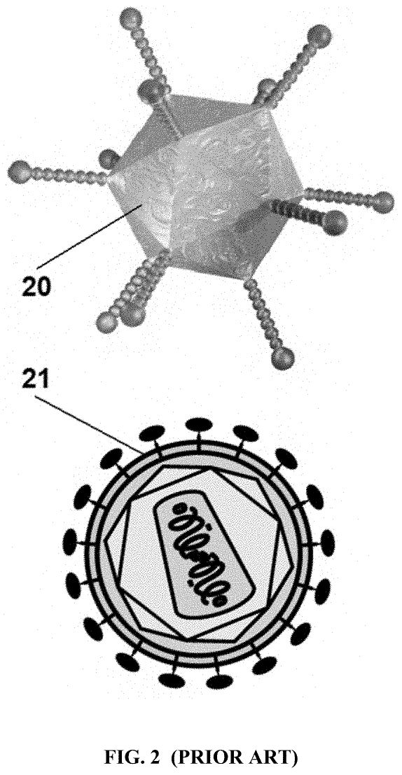

In FIG. 2 are illustrated the Adenovirus and the Human Immunodeficiency Virus, which shows how different in shape and function the viruses can be. Their structure and shape dictate their functionality and the method used to penetrate a cell, which is essential for their multiplication, as seen in FIG. 1. In general, the virions consist of two or three parts: the genetic material (DNA, RNA, etc.), a coat (called capsid) made from viral genome encoded proteins and only for enveloped viruses an envelope of lipids that surrounds the capsid when viruses are outside a cell. The enveloped viruses can be rendered harmless when their viral envelope is destroyed, situation in which the virus no longer has the recognition sites to identify and attach to host cells. On their turn the non-enveloped viruses, their capsid made of proteins is vulnerable to be breached and once the viral genetic material is out, it is easily identified as a foreign invader by the host immune system and destroyed. Thus, the structure and shape of a virus is essential not only for their functionality, but also for finding ways to render them inactive by the immune system, medications, or various vaccines.

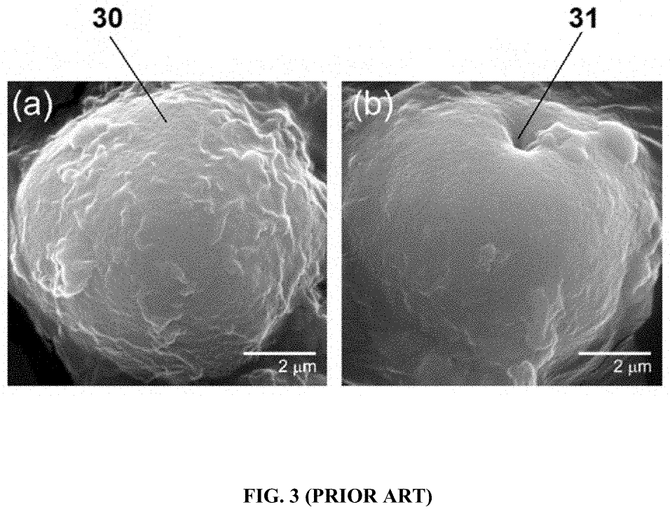

In FIG. 3 is presented the action of shockwaves on mammalian cells 30, from Lopez-Marin L M, et al., “Shock wave-induced permeabilization of mammalian cells”, Physics of Life Reviews, (2018), https://doi org/10.1016/j.plrev.2018.03.001. Lopez-Marin and the colleagues studied the permeabilization of mammalian cells, as a fundamental mechanism to develop gene and cell therapies based on macro-molecular cargo delivery, a process that emerged against an increasing number of health afflictions, including genetic disorders, cancer and infections. Their work shown that the shockwave-induced mechano-sensitive pores 31 in mammalian cells 30 are produced in a safely manner and for sufficient time to allow transfer of genes, drugs, and in general macromolecular delivery into mammalian cells. The study determined that after sixty shockwaves the dyes are able to penetrated through the newly forms pores 31 inside the mammalian cells 30. According to these researchers, the pores 31 can last for few seconds, but the pore-related deformation lasts for minutes. However, once a pore 31 is open into the mammalian cell 30, the subsequent shockwaves can maintain the pores 31 open due to constant variation from positive to negative pressures produced by the shockwaves or pressure waves when passing the mammalian cell 30. These pressures produced by shockwaves or pressure waves also can help with pushing different elements inside the mammalian cell 30, due to the osmotic flow of fluids through a semipermeable membrane that is happening when the fluid travels from an area with a low-solute concentration, to one with a higher-solute concentration. Thus, shockwaves or pressure waves are able to create pores 31 in the mammalian cells 30 and use their pressure variations to facilitate the efficient and rapid delivery of different medicine and vaccines. This was confirmed in cell culture experimentation, where it was shown that after 200 shockwaves there is a 50-fold increase in reporter gene expression per million cells, as mentioned by Shamit Shrivastava, Robin O. Cleveland, and Matthias F. Schneider, “On measuring the acoustic state changes in lipid membranes using fluorescent probes”, Soft matter, vol. 14, pp. 9702-9712, 2018. Besides a rapid intake, due to increase efficiency in pushing the vaccines or different medication inside the mammalian cells 30, the shockwaves or pressure waves can also be used to reduce the actual quantity of medical agent needed for an efficacious effect, which has an important economic impact.

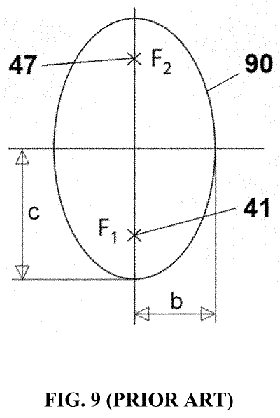

The high mechanical tension and pressures found at the front of the focused acoustic pressure shockwave distinguishes them from other kinds of sound waves, such as ultrasonic waves or pressure waves. FIG. 4 presents the main and unique characteristics for the focused acoustic pressure shockwave 40, which are the same regardless of the principle used to generate them. In the specific case described in FIG. 4, the shockwaves are produced via the spark-gap electrohydraulic principle. To focus shockwaves, it is necessary to produce them in one point and then focus the shockwaves towards another point where their action is needed. The only geometry that has two focal points is the ellipse and in three-dimensional realm is the ellipsoid (see FIGS. 4, 9, 10, 11, 13A-13B, 14C, 15, and 18), which is the geometry used for the reflector 42. However, for the medical field the second focal point F₂ of the ellipsoidal geometry must coincide with the tissue being treated or the region where the shockwaves' action is needed, which means that only a semi-ellipsoidal reflector 42 and not full ellipsoid can be used, as clearly depicted in FIGS. 4, 10, 14C, 15, and 18). Thus, the focused acoustic pressure shockwave 40 are produced via discharging a high voltage in the fluid-filled reflector cavity 43 and in between the first electrode 45A and second electrode 45B of the spark-gap 41, which is placed in first focal point F (FIGS. 9, 13A-13B, 14C, 15, and 18) of the semi-ellipsoidal reflector 42. The high voltage discharge produces an oscillating plasma bubble that creates kinetic energy in the fluid present in the fluid-filled reflector cavity 43. This high energy kinetic energy is in fact the shockwave that is then reflected by the semi-ellipsoidal reflector 42 towards the second focal point 47 (F₂ from FIGS. 9, 13A-13B, 14C, 15, and 18). The shockwave focusing 46 is done towards a focal volume 48 that is centered around the focal point 47 (F₂ of the ellipsoidal geometry). To keep the fluid inside the fluid-filled reflector cavity 43 a coupling membrane 44 is used that stays on top of the opening/aperture of the semi-ellipsoidal reflector 42. In the focal volume 48, produced by the focused acoustic pressure shockwave 40, there is a special pressure “P” profile function of time “t” that defines the shockwave pressure signal 49. Thus, there is a sharp increase in positive/compressive pressure to the maximum shockwave positive pressure 49B of the shockwave compressive phase 49D, which is defined by a rise time 49A in the range of tens of nano seconds to hundreds of nano seconds. After the positive pressure is reaching the maximum shockwave positive pressure 49B, then the pressure decreases exponentially towards zero, which completely defines the full shockwave compressive phase 49D of the shockwave pressure signal 49. The pulse width 49C is defined as the time interval beginning at the first time the positive pressure exceeds 50% of the maximum shockwave positive pressure 49B. The larger the pulse width 49C, the larger and more powerful is the shockwave compressive phase 49D and its influence on cells and tissues. Once the pressures are in the negative values, they are in the shockwave tensile phase 49F. After reaching the maximum shockwave negative pressure 49E, the pressure is going back towards zero to completely outline the shockwave tensile phase 49F profile and also define the end of the focused shockwave pressure signal 49. During the shockwave tensile phase 49F that is characterized by negative pressures, cavitation bubbles can be generated in fluids. The cavitation bubbles are gas voids in fluids that grow as long as the energy is delivered to the bubble. This energy is released from the bubble during its collapse (implosion) in the form of high-speed pressure micro jets and localized/transient high temperature. The micro jets and elevated temperature are present within the shockwave tensile phase 49F and are transient in nature. The compressive pressures, the high-speed pressure micro jets, and localized/transient high temperature occur with each shockwave pulse and all of them are contributing to the in vivo effects on cells and tissues. The total time duration of a shockwave pressure signal 49 is in between five to eight microseconds, which defines a strong and rapid pressure variation, that produces significant, controlled, and efficient effects on cells and tissues during various medical treatments and/or stimulation processes facilitated by the embodiments of these inventions.

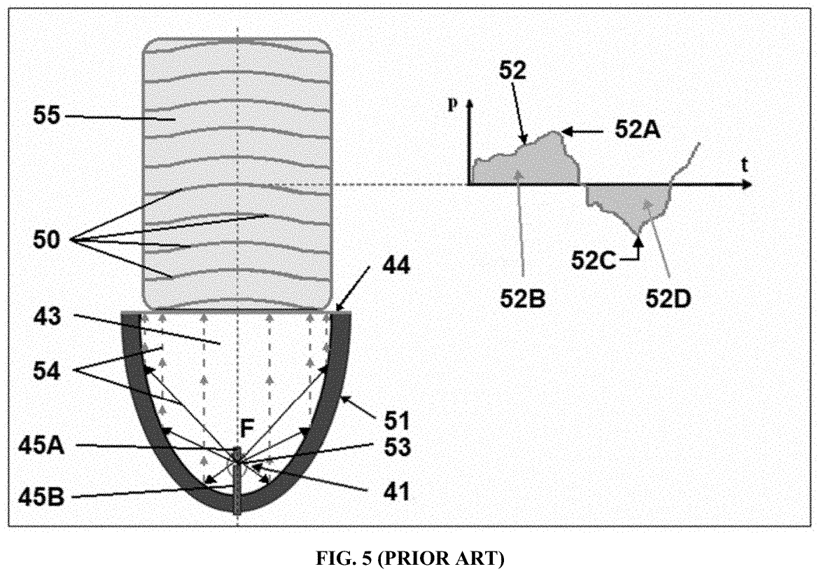

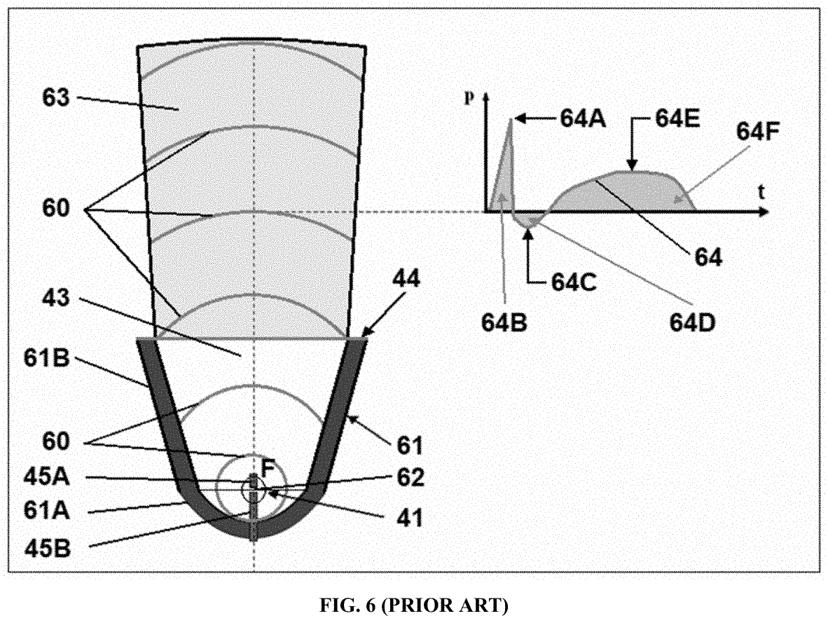

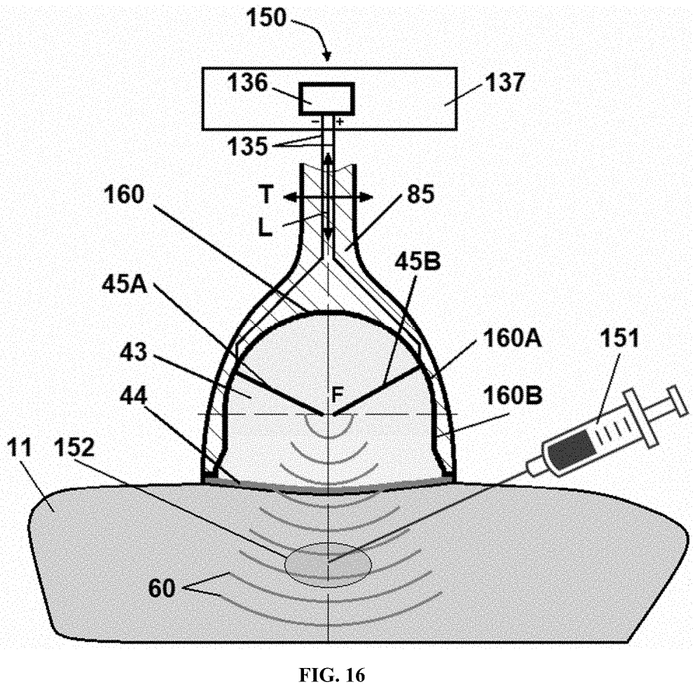

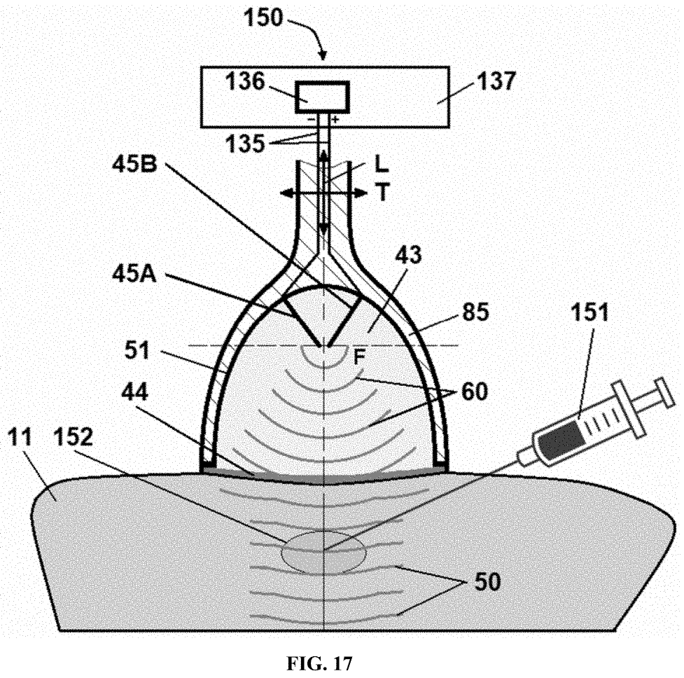

Focused acoustic pressure shockwaves 40 are more powerful in general and deposit more energy in the targeted tissue when compared to pressure waves, which are having a pressure signal flatter and more sinusoidal in shape for acoustic planar pressure wave 234 (FIG. 23) or pseudo-planar pressure wave 50 (slightly distorted planar waves), as presented in FIGS. 5 and 17, and distorted tooth-shape followed by a large positive pressures region for radial pressure wave 60, as seen in FIGS. 6 and 16. Due to lower positive pressures and smaller values for negative pressures for the acoustic planar pressure wave 234 or pseudo-planar pressure wave 50 or radial pressure wave 60, such pressure waves will put less energy inside the targeted cells and tissues, when compared to focused acoustic pressure shockwave 40. However, the acoustic planar pressure wave 234 or pseudo-planar pressure wave 50 or radial pressure wave 60 can cover a larger area of action, which can be advantageous in some situations.

The reflectors used to create pseudo-planar pressure wave 50 are parabolic reflectors 51 characterized by only one focal point known as parabolic focal point 53 (F), which in this situation is inside parabolic reflector 51 as presented in FIGS. 5 and 17. The pressure waves are generated by the high voltage discharge in between the first electrode 45A and second electrode 45B of the spark-gap 41 placed in the parabolic focal point 53 (F), as presented in FIGS. 5 and 17. The high voltage discharge produces an oscillating plasma bubble that creates kinetic energy in the fluid-filled reflector cavity 43, which actually generates the pressure waves. To keep the fluid inside the fluid-filled reflector cavity 43 a coupling membrane 44 is used that stays on top of the opening/aperture of the parabolic reflector 51. These pressure waves are moving radially from the parabolic focal point 53 (F) in the form of radial pressure wave 60 (see FIG. 17) towards the parabolic reflectors 51, which produces the pressure waves reflection 54 parallel to its longitudinal axis (similar to a flash light) and thus creating outside the reflector 51, the pseudo-planar pressure waves 50 inside the pseudo-planar waves pressure field 55 that overlaps with the targeted zone for cleaning and high-level disinfection. In some cases, clean acoustic planar pressure waves 234 are created using planar piezoelectrical crystals or piezo-fibers as presented in FIG. 23. For the acoustic planar pressure waves 234 there is no reflection involved and thus no reflectors are needed. Since the acoustic planar pressure wave 234 or pseudo-planar pressure wave 50 are almost identical in their action and the pressure signal shape generated in the targeted action zone, they will be described together as a bundle. Thus, the acoustic planar pressure wave 234 (FIG. 23) or pseudo-planar pressure wave 50 (FIGS. 5 and 17) are characterized by almost equal maximum planar/pseudo-planar wave positive pressure 52A and maximum planar/pseudo-planar wave negative pressure 52C in absolute values, which makes the planar/pseudo-planar wave pressure signal 52 from the planar/pseudo-planar waves pressure field 55 to have nearly a sinusoidal shape/variation for pressure “P” versus time “t”. This also means that the planar/pseudo-planar wave compressive phase 52B and the planar/pseudo-planar wave tensile phase 52D have similar energy incorporated in them (given by the area in between the curve and time axis).

The wave form of the radial pressure waves 60 is presented in FIG. 6. In general, the radial pressure waves 60 have a duration (more than one thousand microseconds), which is more than 100 times longer when compared to focused acoustic pressure shockwave 40 (less than ten microseconds, or more precise in between five to eight microseconds). When using the spark-gap electrohydraulic principle to generate the radial pressure waves 60, a high voltage discharge in between the first electrode 45A and second electrode 45B of the spark-gap 41 is produced in the sphere central point 62 (F) of the combination semi-spherical and conical reflector 61. The high voltage discharge produces an oscillating plasma bubble that creates kinetic energy in the fluid-filled reflector cavity 43. To keep the fluid inside the fluid-filled reflector cavity 43 a coupling membrane 44 is used that stays on top of the opening/aperture of the combination semi-spherical and conical reflector 61. To not impede with the propagation of the radial pressure waves 60 generated in the sphere central point 62, the combination semi-spherical and conical reflector 61 has a conical reflector portion 61B towards its mouth. The semi-spherical reflector portion 61A reflects the radial pressure waves 60 (propagating towards the bottom of the combination semi-spherical and conical reflector 61) back towards the sphere central point 62. That means that they are not contributing to the radial pressure waves 60 present in the radial waves pressure field 63 that overlaps with the targeted zone for cell or tissue treatments and/or stimulation. However, the reflected portion of the radial pressure waves 60 from the bottom of the combination semi-spherical and conical reflector 61 towards the sphere central point 62 will collapse any residual bubbles left in the spark-gap 41 area from the plasma bubble generated by the high voltage discharge in the fluid-filled reflector cavity 43, which will allow the subsequent radial pressure waves 60 to be generated into a pretty consistent way. In the radial waves pressure field 63, the radial pressure wave 60 are characterized by a radial wave pressure signal 64 that has distorted tooth-shape followed by a large positive pressure region. Thus, there is a sharp increase in positive/compressive pressure to the maximum radial wave positive pressure 64A of the radial wave compressive phase 64B. After the positive pressure is reaching the maximum radial wave positive pressure 64A, then the pressure decreases almost instantaneous towards zero, which completely defines the full radial wave compressive phase 64B of the radial wave pressure signal 64. Once the pressures are in the negative values, they are in the radial wave tensile phase 64D. After reaching the maximum radial wave negative pressure 64C, the pressure is going back towards zero to completely outline the radial wave tensile phase 64D profile. However, the pressures continue to increase and become positive again and reach maximum remnant positive pressure 64E, which is smaller when compared to the maximum radial wave positive pressure 64A. After the positive pressure is reaching the maximum remnant positive pressure 64E, then the pressure decreases slowly towards zero, which completely defines the full remnant positive pressure phase 64F and the end of the radial wave pressure signal 64. The positive pressure phase 64F incorporates a significant portion of the radial wave pressure signal 64 and also collapses prematurely the cavitation bubbles generated in the radial wave tensile phase 64D, which means that the role played by cavitation is reduced when compared to the focused acoustic pressure shockwave 40 and pseudo-planar pressure wave 50. However, some cavitation is still produced and the double positive pressures (maximum radial wave positive pressure 64A and maximum remnant positive pressure 64E) are still producing good cell or tissue treatments and/or stimulation.

FIG. 24A presents the wave form of the ultrasound waves 241 seen in FIG. 24B. In general, the ultrasound waves 241 have two components. The ultrasound longitudinal wave 247 is the one moving in the ultrasound direction of propagation 249. Adjacent layers of fluid are subjected to a cyclic compression and expansion with velocity dependent on propagation media (liquid, air or solid). Coexisting with the ultrasound longitudinal wave 247 is the ultrasound transversal wave 248, which is a low velocity and high damping wave with a sinusoidal variation in a direction perpendicular to the ultrasound direction of propagation 249. Usually, the ultrasound transversal wave 248 can produce friction in propagation media and consequently possible heat. However, the low-frequency ultrasound has a large ultrasound wavelength 247A that is characterizing the ultrasound cycle 248B and this is why the effects of the ultrasound transversal wave 248 are less pronounced, which means that negligible or no heat is produced. Other important parameters that define the ultrasound waves 241 are the ultrasound maximum positive pressure 248C and ultrasound maximum negative pressure 248D, which are equal in absolute value that is also known as ultrasound amplitude 248A. The positive pressures 248C produce compressive forces/stresses and the negative pressure 248D generate cavitation bubbles. Due to continuous sequence of phases for ultrasound waves, the pressures are changing from positive pressures to negative pressures and then again to positive pressures in a sinusoidal variation, which has a significant influence on the cavitation. Thus, the cyclical acoustic wave of the ultrasound, makes the cavitation bubbles to grow and then collapse due to incoming new positive pressures in a cyclical way too. In general, the ultrasound cavitation bubbles need many ultrasound cycles to reach a dimension that allow them to collapse by themselves. Usually, the ultrasound cavitation bubbles do not reach the same size as the shockwave cavitation bubbles, which translates in less energy generated during their collapse.

In conclusion, as seen from FIGS. 4, 5, 6, 23, and 24A, the focused acoustic pressure shockwaves 40 behave similarly to other sound waves (acoustic planar pressure wave 234 or pseudo-planar pressure wave 50 or radial pressure wave 60 or ultrasound waves 241), with the main difference that the focused acoustic pressure shockwaves 40 possess more energy. A focused acoustic pressure shockwave 40 can travel large distances easily (based on the amount of energy put in them at the point of origination), as long as the acoustic impedance of the medium remains the same. The same acoustic impedance principle is valid for pressure waves (acoustic planar pressure wave 234 or pseudo-planar pressure wave 50 or radial pressure wave 60) and low-frequency ultrasound waves 241, with the caviar that their energy is smaller when compared with focused acoustic pressure shockwaves 40, which might translate in less traveling distance/penetration inside the human or animal or plant body. At the point where the acoustic impedance changes, energy is released and the focused acoustic pressure shockwaves 40 or pressure waves (acoustic planar pressure waves 234 or pseudo-planar pressure waves 50 or radial pressure waves 60) and low-frequency ultrasound waves 241 are reflected or transmitted with attenuation. Thus, the difference in between shockwaves and pressure waves or ultrasound is the amount of energy they deposit inside the targeted zone and sometimes their penetration inside the same targeted zone. Shockwaves are more powerful in general and have more energy due to their higher compressive pressures produced in the compressive phase and larger negative pressure from the tensile phase, which can produce more powerful cavitation bubbles in a fluid (see FIGS. 4, 5, 6, and 24A). On their turn, the pressure waves and low-frequency ultrasound are having a pressure signal flatter, more sinusoidal in shape, and due to their lower positive pressures and smaller values for negative pressures that influences the size of cavitation bubbles, they will put less energy inside the targeted zone.

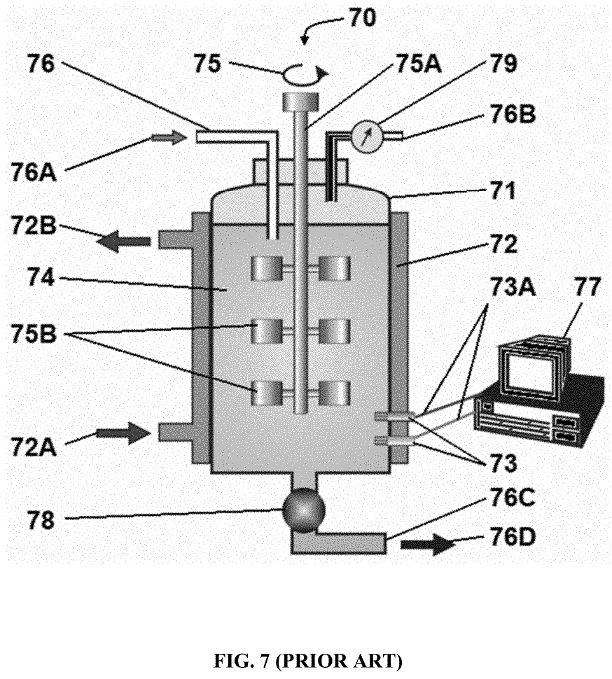

In general, the cells or bacteria or fungi are grown in a nutrient reach medium in a bioreactor system 70, as presented in FIG. 7. The bioreactor is an apparatus for growing organisms (yeast, bacteria, or animal cells) under controlled conditions. Inside the bioreactor biological reactions, as cellular or enzymatic immobilization, are carried out by the cultured aerobic cells. Bacteria can be also multiplied exponentially in a bioreactor, as it is the case for the mRNA vaccines, in order to extract the DNA plasmids (containing a specific virus gene) from multiplied bacteria that are later used to transcribe the specific strands of viral DNA into mRNA. Generally, the major responsibilities of a bioreactor are to provide a biomechanical and a biochemical environment that controls nutrient and oxygen transfer to the cells or bacteria and metabolic products from the cells or bacteria. The bioreactor primary function is to monitor and control the critical process parameters within the vessel (e.g., pH, culture temperature, dissolved oxygen tension, and media exchange). During the bioprocess, the bioreactor feeds a sterile gas mixture such as air or oxygen into the culture medium. Constant stirring not only distributes the nutrients, but it also reduces the size of the gas bubbles that arise in the culture vessel, thus efficiently releasing oxygen or air or other type of gas into the nutrient solution.

Vaccines are biologics to provide immunity, typically against infectious diseases such as that caused by the COVID-19 vaccine usually contains parts of a disease-causing microorganism such as surface proteins or a weakened (attenuated) or killed microorganism. These biological agents stimulate the body's adaptative immune system to recognize them as threats such that the body can destroy the actual microorganism it encounters in the future.

To date the majority of the vaccines are produced from chicken eggs. The candidate vaccine viruses are injected into fertilized chicken eggs and allowed to replicate. Following that, the viruses are harvested from the fluids in the eggs, purified, and ready to use. However, this method has huge limitations in terms of scalability, environmental footprint, and vaccination potency. Each fertilized chicken egg can produce only approximately 100-300 vaccine doses and these eggs must come from special pathogen-free chickens. During times like the COVID-19 pandemic, such an approach was not likely to meet surges in global demand for vaccines. This is why the cell-based vaccines were developed in the effort to meet the increased demand for vaccines.

Faced with limitations of egg-based vaccine production, researchers have turned to other approaches. Instead of chicken eggs, the candidate vaccine viruses are injected into cultured mammalian cells. The virus then hijacks the cell's machinery to replicate many copies of itself. Viruses are finally purified from extracts of cell cytoplasm. There are several benefits of using mammalian cells for vaccine production. For instance, unlike egg-based methods, viruses cultured in mammalian cells are likely to retain mutations that confer them advantage in infecting human cells. This makes them more antigenically matched and useful as human vaccines. Additionally, the quality of mammalian cells can be more consistently monitored using modern biomedical technologies, unlike eggs. In other cases, the multiplying bacteria is used to multiply some genetic material extracted from viruses or cells, which ultimately will be used to create new type of vaccines and genetic medicines. The bacteria do not contribute with any of its own genetic material to the final product, since rigorous filtration and separation is done to collect only the desired genetic material that has a role in creating the drug, medication, vaccine, genetic medicine, etc.

Moving from the traditional egg-based method to mammalian-cell-based vaccine or genetic-based vaccine manufacturing has its challenges. As a result, bioreactor design has evolved to meet the technical requirements for cell-based vaccine manufacturing. The same stands for the case when bacteria are used to multiply the genetic material in the form of plasmids (a genetic structure in a cell that can replicate independently of the chromosomes, typically a small circular DNA strand in the cytoplasm of a bacterium or protozoan). The DNA is then used to transcribe into mRNA that is actually used in the vaccine.

In any biological system, there is always a risk of contamination, which can adversely affect the quality, and more importantly, safety of the products. Many bioreactors now offer closed-system operation as opposed to conventional open-system bioreactors, to ensure the sterility of their contents. Oxygen, air, and nutrients can be introduced to the broth while waste such as cell extracts can be removed from the culture through filtered pipes. Compared to open-system counterparts, closed-system bioreactors reduce the risks of fluid splashing and accidental transfer of contaminants from the outside environment into bioreactors and better protect the operators. They also decrease machine down time, leading to cost savings.

Single-use disposable plastic bag bioreactors are also becoming more popular compared to multi-use stainless steel bioreactors, especially at the initial testing phase. This is because single-use bioreactors are usually a cheaper investment and are available in lower volumes, enabling manufacturers to test different cell lines and growth conditions quickly and at a lower cost to optimize vaccine production. Importantly, as manufacturers may also be testing and producing human and animal vaccines at the same time, single-use bioreactors can minimize potential cross-contamination.

Another important aspect of using bioreactors for biomanufacturing is sensing. To achieve high yield, host mammalian cells or bacterial cells must be healthy to support virus replication or genetic material. When bacteria are used, they must be healthy and be able to multiply vigorously, and thus multiplying the genetic material incorporated in them. Cells or bacteria, being biological agents, are highly sensitive to their environment. Having good chemical sensors measuring temperature, oxygen, pH, nutrients such as glucose and amino acids, and cell or bacterium waste is thus crucial. Sensors are generally more reliable in multi-use bioreactors than single-use bioreactors. This is because expensive and more accurate pH electrodes are generally not incorporated into disposable single-use bioreactor bags. To minimize per-use cost while taking into account the bag flattening assembly and delivery process, single-use bioreactors are typically using non-invasive methods like electromagnetic waves sensing with radio-frequency identification tags, and optic fibers embedded into patches to detect for chemical changes in their cellular contents.

The growing popularity in the use of mammalian cells or bacteria for vaccine production and the development of new stem cell therapies is likely to increase the use of bioreactors. Such bioreactor system 70 is presented in FIG. 7. The bioreactor 71 is surrounded by a bioreactor mantle 72, which is used to control temperatures (via heated or cooling water) inside the bioreactor 71 or after the whole manufacturing process is finished, to sterilize the interior of the bioreactor 71 via steam circulated into the bioreactor mantle 72. For the heating and cooling water or steam circulation in the bioreactor mantle 72, the heating/cooling water “IN” 72A and heating/cooling water “OUT” 72B are used. The probes or sensors 73 are monitoring internal processes inside the bioreactor 71 via pH, culture temperature, dissolved oxygen tension, and media exchange measurements. The connection of the probes or sensors 73 to the computer and control console 77 is done via connection cables 73A. The bioreactor nutrient broth 74 from inside the bioreactor 71 is subject to a stirring motion 75 via the stirring shaft 75A and paddle stirrers 75B. The initial introduction of the bioreactor nutrient broth 74 (can contain cells, bacteria, yeast, fungi, etc.) inside the bioreactor 71 is done via inlet port 76. The same port is used during the batch processing to feed the bioreactor nutrient broth 74 with nutrients, air, microbes, or other elements needed (microbes, air, or nutrients “IN” 76A). Excessive gas is eliminated from inside the bioreactor 71 via the gas outlet 76B. The monitoring of the gas pressure inside the bioreactor 71 is done via the pressure gauge 79. The desired product is collected at the bottom of the bioreactor 71 via the product “OUT” 76D associated with the product outlet 76C. Usually, this process involves a filtration process that can be included in the product outlet 76C or can be done separately in a dedicated filtration system. To control the collection of the product the valve 78 is used on the pipe that goes to the product outlet 76C. The computer and control console 77 is monitoring and controlling all the processes, inputs, outputs of the bioreactor 71, via artificial intelligence (AI) algorithms, to produce an optimal cell culture environment and ultimately the best quantity and quality of the output product (vaccine, medication, genetic medicines, stem cells, or immune cells, to name a few).

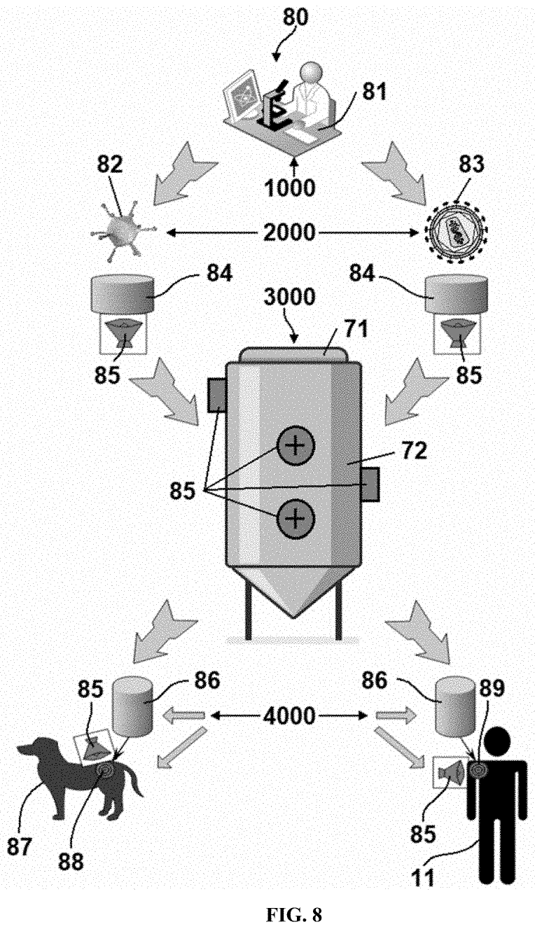

Referring to FIG. 8, this embodiment presents the use of bioreactors in combination with shockwaves (focused or unfocused) or pressure waves (planar, pseudo-planar, radial, or ultrasound) for pharmaceuticals, gene medicines, stem cells, vaccines, or antibodies that are produced via a cellular or bacterial culture. The process is mainly discussed for production of vaccines, although the same process can be employed for other types of output products as pharmaceuticals, gene medicines, stem cells, or antibodies, to name a few. For vaccines, the cell cultures are infected with viruses, which reproduce and serve as the basis of a vaccine. To obtain the maximum possible density of host cells, researchers have to manipulate numerous factors. If bacterial cells are used for multiplication of genetic material for mRNA vaccines, similar process steps are applied as for the cellular cultures, with minor specific modification.

Despite the huge increase in development of cells, gene therapies, and the newest genetically modified vaccines types, over the past couple of years, manufacturing technology for these therapies is largely still at the first-generation stage. Often cell and gene therapy manufacturing processes are highly manual, which is stemming from the early academic or process development stage. These first-generation processes cause manufacturing to be expensive and with low-throughput, which reduces the ability of patients to access these potentially life-saving therapies.

In addition to making the process quicker, cheaper, and more accurate, computing tools can also help with quality control and tracking. In cell therapy manufacturing, especially autologous products, line of sight around electronic batch records, as well as the vein-to-vein supply chain, is incredibly important. The realm of cell manufacturing not only in medical field but also in industrial and food biotech must apply rapid improvements in advanced computing options such as artificial intelligence (AI) and machine learning technology, as well as robotics to enhance the growth of microbial strains at a commercial scale, which fits perfectly with the shockwave or pressure waves or ultrasound technologies that can be used to stimulate cellular or bacterial cultures in a bioreactor. The use of AI for shockwave or pressure waves or ultrasound technology for medical purpose is mentioned in U.S. Pat. No. 10,888,715.

Besides being sensitive to their chemical environment, cells and bacteria are also affected by mechanical forces in their environment. The cells and bacteria growing at the bottom of static bioreactors experience more hydrostatic pressure, which can affect their growth. Additionally, excessive fluid shear stress in a stirred-tank bioreactor can also negatively affect cellular and bacterial functions. To optimize mechanical stresses while providing sufficient agitation for uniform distribution of nutrients, companies have introduced new designs such as rocking bioreactors. The rocking motion of the platform induces waves to mix and transfer oxygen to the culture medium to create an optimal environment for cell or bacterium growth. For adherent mammalian cells and bacteria, micro-carriers, usually polymeric beads, are also being used to culture these cells and bacteria in bioreactors to optimize space use, maximize surface area to volume ratio to promote cell or bacterium growth, and reduce unnecessary mechanical stresses.

The vaccine production process 80 presented in FIG. 8, is based on a series of steps that start with the viral agent selection step 1000, continues with bench proliferation step 2000 that is followed by the bioreactor proliferation and filtration step 3000. Finally, after the vaccine production, the vaccination step 4000 shows the importance of shockwaves or pressure waves or ultrasound in the efficient delivery of the vaccine. The viral agent selection step 1000, bench proliferation step 2000, the bioreactor proliferation and filtration step 3000, and the vaccination step 4000 may employ the shockwave or pressure wave or ultrasound applications and devices in various ways and methods as presented in these inventions. Shockwaves or pressure waves or ultrasound can be used to stimulate cellular proliferation and/or bacterial proliferation at different stages using different constructions, settings and overall functionality of the shockwave/pressure wave device 85. The use of shockwaves or pressure waves or ultrasound devices (devices 85) in different phases of the vaccine production or for vaccine inoculation represents a faster and cheaper approach for enhancing productivity when compared to conventional technologies that are employed right now.

The viral agent selection step 1000 is the step where the research station 81 is used to determine the right approach for vaccine development and select the type of viral particle or viral particles (virus “A” 82 or virus “B” 83) that will be at the core of the development for the respective vaccine. The experimentation is done with classic cellular, bacterial, and viral assessing methods combined with stimulation of the cells or bacteria, to enhance their production of viral particles or genetic material, via shockwaves or pressure waves or ultrasound, which can be done using fixtures similar to the embodiment presented in FIGS. 14A and 14B.

Once the viral agent selection step 1000 is done, the selected type of cells or bacteria for culture and viral agents needed for the vaccine must be scaled up via initial bench proliferation step 2000. This step involves manual work to produce sufficient quantity of intermediary cellular or bacterial culture 84 in culture plates, tube welding and transfers from flask to bag to bigger bags, to be able to create a sufficient quantity that is transferable to a bioreactor 71. The intermediary cellular or bacterial culture 84 that sits in either containers or bags can be stimulated for higher output using the shockwave/pressure wave device 85 that can produce shockwave or pressure waves or ultrasound waves. It is important to note that the pressure waves category for these devices 85 includes planar or pseudo-planar or radial or unfocused waves or ultrasound pressure waves.

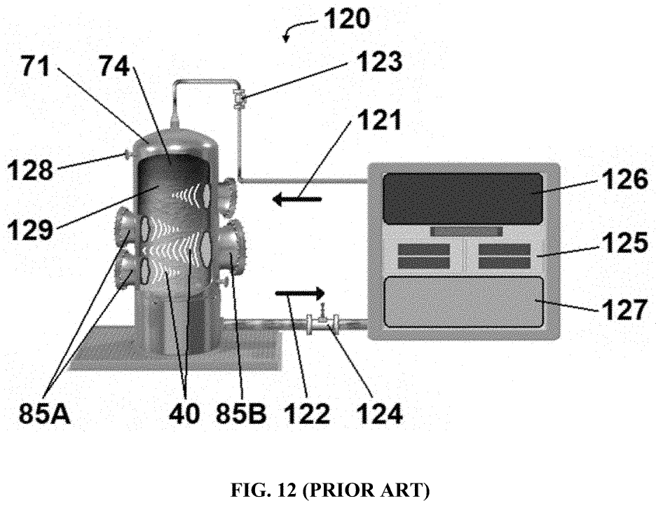

When enough quantity of intermediary cellular or bacterial culture 84 is produced via cell or bacterium proliferation using shockwave/pressure wave device 85, then the bioreactor proliferation and filtration step 3000 is ready to start. In this case, multiple shockwave/pressure wave device 85 are integrated into the bioreactor 71. The shockwave/pressure wave device 85 are designed in such way that allow a rapid exchange in case of failure and do not compromise the batch of cellular, or bacterial culture from inside the bioreactor 71. For that they need to stand-out from the bioreactor mantle 72, as seen in FIG. 8. More details of the integration of the shockwave/pressure wave device 85 with the bioreactor 71 can be seen in FIG. 12. In the same FIG. 12 is seen that the shockwave/pressure wave device 85 can be of different sizes and placed with a certain algorithm all around the bioreactor 71 to produce an effective stimulation and mixing of all the cellular or bacterial culture, for an efficient and productive vaccine process. In both FIGS. 8 and 12, the shockwave/pressure wave device 85 are placed on circumferential positions at different heights, which allows a uniform mixing and stimulation of the cellular or bacterial culture and in the same time reduces the size of the gas bubbles that arise. It is important to mention that the stirring of cellular or bacterial culture with shockwaves or pressure waves or ultrasound (via acoustic streaming or acoustic microstreaming) is much gentler when compared to the paddle stirrers 75B from FIG. 7, which avoids unnecessary death of cells or bacteria due to excessive shear forces created by the paddles. The details of the characteristics and the actions of the shockwave or pressure wave or ultrasound devices 85 inside the bioreactor 71 are similar to all embodiments presented throughout these inventions.

The use of shockwaves or pressure waves or ultrasound for bioreactors 71 can also minimize the number of manual steps needed to produce a given vaccine, stem cells, immune cells, medication, or gene medicine, which speeds up the process as well as making it more accurate. Another advantage is that the manufacturer can tailor the production capacity according to demand.

Once the bioreactor proliferation is finished, the actual vaccine or genetic material or specific cellular or protein material, etc. is collected, filtrated to completely finish the bioreactor proliferation and filtration step 3000. Interesting to note that the same shockwaves or specifically modulated pressure waves or ultrasound can also play a role in the membrane filtration processes, or in the separation processes for vaccines or vaccine components, since the shockwaves or pressure waves or ultrasound produce acoustic streaming and microstreaming that are pushing particles in preferred directions (unidirectional), which overlap with the longitudinal direction for propagation of the shockwaves or pressure waves. The filtration can be tangential or perpendicular to the filtration or separation membrane used for the process and the shockwaves or pressure waves or ultrasound can handle both tangential and perpendicular direction of the flow through the filtration or separation membrane. Furthermore, the shockwaves or pressure waves or ultrasound can be used to unclog the filtration or separation membrane and thus prolonging its useful life.

After the bioreactor proliferation and filtration step 3000 the vaccine material is separated in vaccine doses/shots 86 that are ready for vaccine injection of the vaccination step 4000. Vaccines can be used for humans, animals and plants, with most of the vaccines being available for animal vaccination 88 and human vaccination 89, as presented in FIG. 8. For the animal vaccination 88, the prepping of the exact location on the body of the animal 87 is done with the shockwave/pressure wave device 85 before or during the vaccine injection. In this case, the shockwave/pressure wave devices 85 have smaller dimensions, to fit the dimension of the animal 87 and properly stimulate the pores opening on cellular membranes only in the desired area. This will assure an efficient and rapid intake of the vaccine dose/shot 86. For the human vaccination 89, usually the vaccines are administered in certain locations of the human body 11, as arms, legs, buttocks, etc. All these parts of the human body 11 allow the safe use of the shockwave/pressure wave device 85 to stimulate the rapid and efficient intake of the vaccine dose/shot 86. For human vaccination 89, the shockwave/pressure wave devices 85 should also have slick designs and smaller dimensions, to stimulate a region that matches the injection site. Also, the use of shockwaves or pressure waves or ultrasound on the vaccine injection site may also reduce the inflammation and pain that might occur after vaccination, since the shockwaves or pressure waves or ultrasound have proved in scientific studies that they can modulate the inflammation (reducing its duration) and decrease or eliminate pain via analgesic effects that were demonstrated in treating orthopedic conditions or various acute and chronic wounds.

Although the exemplification was for vaccine production, the embodiment presented in FIG. 8 can be also used for production of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

FIG. 9 presents the typical ellipsoidal geometry that is used for focusing shockwaves. The ellipse 90 is the only geometry that has two focal points, which means that whatever is generated in the first focal point F₁ can be reflected and focused in the second focal point F₂. Based on this property, if a form of energy carrier is produced in F₁ via a high voltage discharge in a fluid using a spark-gap 41, it can be reflected and finally concentrated in the second focal point F₂, defined as the focal point 47. That is practically the way the focused acoustic pressure shockwaves 40 are generated and focused towards a targeted region that sits around the second focal point F₂ (focal point 47). The elliptical geometry is characterized by the major elliptical semiaxis “c”, minor elliptical semiaxis “b”, and their ratio, which dictates the actual ellipse length and width, with significant implications on the reflective properties of a potential reflector with that specific geometry and in the end on the actual penetration inside the human or animal body of the shockwaves or pressure waves or ultrasound.

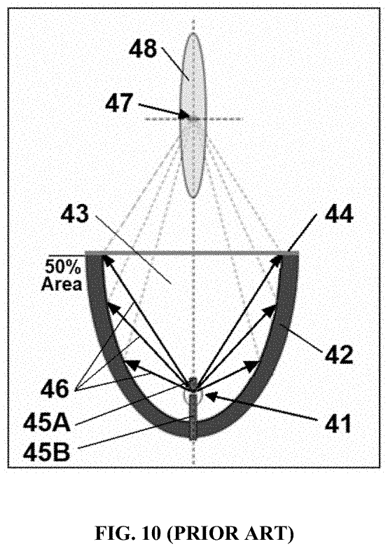

FIG. 10 is a geometric representation of the classic semi-ellipsoidal reflector 42, which is usually used to produce reflect focused electrohydraulic shockwaves. Since the main action of the focused acoustic pressure shockwaves 40 is produced in the focal volume 48 that encompasses the second focal point F₂ (defined as focal point 47), only a portion of the ellipsoidal geometry can be used for focusing. In most of the cases half of the ellipsoidal geometry can be used and those are the classic semi-ellipsoidal reflectors 42. When the targeted region is placed before or after the focal volume 48, then the treatment is produced by unfocused pressure waves. The semi-ellipsoidal reflector 42 can be shallow or deep, based on the ratio of the major elliptical semiaxis “c” and minor elliptical semiaxis “b”. If the ration c/b is higher than 1.9 (c/b>1.9), then the semi-ellipsoidal reflector 42 is considered to be deep. With a ratio of 1.1≤c/b≤1.3 the semi-ellipsoidal reflector 42 is considered shallow and for 1.4≤c/b≤1.9 is considered normal based on an optimal penetration inside the human body 11 to reach the structures under the skin down to the arterial circulation. The deeper reflectors will produce shockwaves that will penetrate deeper inside the human body 11 and conversely the shallow reflectors will have a superficial penetration. The first focal point F₁ of the semi-ellipsoidal reflector 42 is where the shockwaves are generated via spark-gap 41 for the electrohydraulic principle that uses two electrodes (first electrode 45A and second electrode 45B) to produce a high voltage discharge in a fluid. The shockwave focusing 46 is done by the internal surface of the classic semi-ellipsoidal reflector 42 towards the focal point 47 (second focal point F₂) and the surrounded focal volume 48, as presented also in FIG. 4. To keep the fluid inside the fluid-filled reflector cavity 43 a coupling membrane 44 is used that stays on top of the opening/aperture of the semi-ellipsoidal reflector 42. These elements and construction presented in FIGS. 9 and 10 for the semi-ellipsoidal reflector 42 will be found in many of the embodiments presented in FIGS. 12, 14A, 14B, 15, and 18.

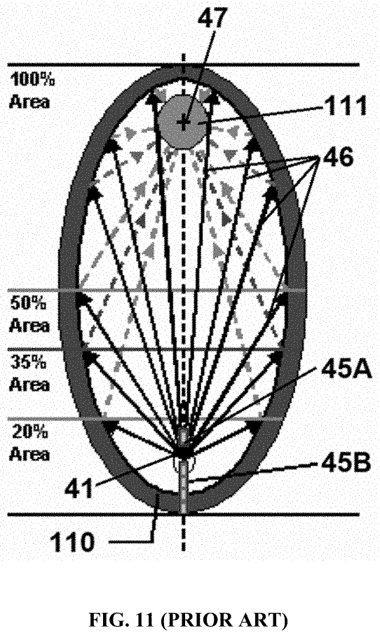

FIG. 11 presents a full ellipsoidal reflector 110 and the way the shockwave focusing 46 is produced towards the focal point 47. Thus, if a three-dimensional reflector is created in the form of an ellipsoid, the pressure shockwaves generated in the first focal point F (where the spark-gap 41 is found) will be reflected with minimal losses by the full ellipsoidal reflector 110 in the second focal point F₂, also known as focal point 47. In this situation, the shockwave focusing 46 is done with the whole ellipsoidal surface, which is different from the semi-ellipsoidal reflector 42 (see FIG. 10), when only half of the ellipsoid is used, where some portions of the shockwaves are not reflected towards the focal point 47 and they continue to propagate divergently away from the focal point 47. This is why a full ellipsoidal reflector 110 had a high efficiency in focusing all shockwave fronts from all directions and collect their energy right around the focal point 47. Due to shockwave focusing 46, a focal volume 111 is created around F₂ (the focal point 47), which has a spherical shape. In the focal volume 111 is where the maximum shockwave positive pressure 49B are found from the shockwave compressive phase 49D that produces macro effects, together with micro-jets (micro-effect) produced by the collapsing of the cavitation bubbles generated in the shockwave tensile phase 49F, as seen in FIG. 4. In FIG. 11 the shockwaves are produced in a fluid by the high voltage discharge in the spark-gap 41 that is formed by the first electrode 45A and second electrode 45B, which represents the spark-gap electrohydraulic way to produce shockwaves. Other ways to produce shockwaves are using the electromagnetic and piezoelectric principles.

In general, the amount of energy delivered to a targeted region by the shockwaves is directly proportional with the surface area of the reflector. As presented in FIG. 11, in medical pressure shockwave applications the reflectors represent only a percentage of a full ellipsoid (in between 20 to 50% of the area). This is why a full ellipsoidal reflector 110 that is using all its surface for reflection has the advantage of being more efficient in stimulation of cellular cultures or bacterial cultures via constant pressure gradients generated uniformly inside the whole volume of the full ellipsoidal reflector 110. Also, the moving of the shockwaves from spark-gap 41 towards the focal point 47 and the shockwave focusing 46 create sufficient acoustic streaming and acoustic micro-streaming that assures the gentle mixing of the cellular culture or bacterial culture.

In similar embodiments to the one presented in FIG. 11, the full ellipsoidal reflector 110 can be replaced by two parabolic reflectors 51 (see FIG. 5) that together form a full enclosure that generate shockwaves or pressure waves from two different points, one from the top that corresponds to focal point 47 from FIG. 11, which preferably may be the focal point of the first top-positioned parabolic reflector 51, and the second point from the bottom that corresponds to spark-gap 41 from FIG. 11, which preferably may bathe focal point of the second bottom-positioned parabolic reflector 51. This embodiment preferably may balsa capable to efficiently stimulate the cellular cultures or bacterial cultures found inside the full enclosure formed by two parabolic reflectors.

Making billions of doses of vaccine is a herculean task. The tools needed for manufacturing a vaccine vary considerably depending on the kind of vaccine, but in many cases, a bioreactor is needed, which is a giant tank that allows the growing of organisms that are actually spewing out the vaccine of interest or genetic material or proteins or any component needed for the production of a vaccine. The capacity of such bioreactors varies from few galloons/tens of liters to a 20,000-gallon/75,000-liter volume capacity. This is specialty equipment that has to be made. With the newly developed processes for the mRNA or DNA vaccines, the task becomes even more complicated, since every step is new. Also, new drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like, all require new type of processes and means to improve and speed-up the manufacturing processes, which generate changes in the bioreactors' construction. Shockwaves or pressure waves or ultrasound can be easily applied to small cell/bacterium cultures or inside the bioreactor with dedicated shockwave or pressure wave or ultrasound devices 85 and even the actual bioreactor can be constructed in the shape of a large ellipsoid (see the full ellipsoidal reflector 110 from FIG. 11) or by combining two parabolic reflector 51 (as mentioned in the previous paragraph) and shockwaves or pressure waves applied to all the mass inside the bioreactor to take advantage of the high efficiency effects of shockwaves or pressure waves in stimulation and mixing cell cultures or bacterial cultures from inside the bioreactor.

All bio-based therapeutics is sourced from a live cell or a component of one. Most gene therapies are built on viruses found in nature. The more complicated the biologic becomes, the more parts of it require optimization, and the more analytics are required to control the bioreactors' processes. There is also a lot of waste in cell therapy manufacturing and yields are impaired by high cell death at every passage mainly due to the shear forces produced by the stirring mechanism of the bioreactors. Paddle-induced shear stress is damaging the cells, thus negatively impacting cell viability and triggering undesired genetic mutations. Similar effects are seen when bacterial cultures are used inside the bioreactors. This is where the use of shockwave or pressure waves or ultrasound to stir the cell culture could be significantly beneficial, due to the elimination of the high shear forces, which are replaced by the acoustic streaming or microstreaming that are much gentler to the cell culture.

For a uniform delivery of the shockwaves or pressure waves inside the bioreactor multiple shockwave/pressure wave devices 85A and 85B must be used, as seen in FIG. 12. These multiple devices can function intermittently at different time points or in the same time for a predetermined time period and then followed by a period of silence. The embodiment presented in FIG. 12 is an automatic bioreactor system 120 that uses a multitude of small shockwave/pressure wave device 85A and large shockwave/pressure wave device 85B that are positioned with a certain algorithm all around the cylindrical enclosure of the bioreactor 71. The shockwaves or pressure waves generated inside the bioreactor 71 are capable to stimulate the growth of cells or bacteria found in the cellular or bacterial culture 129 and in the same time to mix the same culture in a gentle way using acoustic streaming and acoustic microstreaming produced by the focused acoustic pressure shockwaves 40 or pressure waves (not shown in FIG. 12 that exemplifies the embodiment that is using shockwaves). The automatic bioreactor system 120 uses a dedicated cellular or bacterial culture compartment 126 from where the cellular or bacterial culture introduction 121 is done at the top of the bioreactor 71. The periodic introduction of the cellular or bacterial culture 129 inside the bioreactor 71 is controlled via the control console/panel 125 that opens or closes the cellular and bacterial culture valve 123. During stimulation of the cellular or bacterial culture 129 that is mixed with the bioreactor nutrient broth 74, the original combined volume is used until the end of the process and in other situations new volumes of the cellular or bacterial culture 129 can be introduced periodically. Once a batch or certain volume of cellular or bacterial culture 129 is inside the bioreactor, then the shockwave or pressure wave applicators/devices 85A and 85B are actuated. The small shockwave/pressure wave device 85A generate less energy when compared to the large shockwave/pressure wave device 85B. This different energy output feature combined with specific settings for each shockwave or pressure wave applicators/devices 85A and 85B, allows a fine tuning of the stimulation or mixing parameters, to adapt to each possible type of cellular or bacterial culture 129. The selection for actuation of the shockwave or pressure wave applicators/devices 85A and 85B and for their functional parameters can be done with specific artificial intelligence (A/I) algorithms that can adapt in time based on each situation and type and volume of cellular or bacterial culture 129. The functioning parameters for the bioreactor 71, the cellular or bacterial culture 129 metabolism, and the monitoring of internal processes inside the bioreactor 71 via pH, culture temperature, dissolved oxygen tension, and media exchange measurements are done via probes or sensors 73, as presented in FIG. 7 (not shown specifically in FIG. 12). However, to access the cellular or bacterial culture 129, these probes or sensors 73 use probe or sensor ports 128 situated at the top or bottom or middle of the bioreactor 71, as presented in FIG. 12. The stimulation of the cellular or bacterial culture 129 is done for multiple days until the desired number of vaccine elements needed to produce the vaccine is accomplished. At that time point, the stimulation and mixing of the cellular or bacterial culture 129 via the shockwave or pressure wave applicators/devices 85A and 85B is stopped and the vaccine component collection 122 is done from the bottom of the bioreactor 71 and the storing of the solution with the vaccine components is done into the vaccine component solution compartment 127. The control console/panel 125 controls the vaccine component collection 122 via the opening or closing of the vaccine component valve 124. Regardless if the bioreactor 71 process is continuous or discrete, it is necessary to have periodic cleaning, disinfection, sterilization and maintenance. For that the bioreactor 71 must emptied, washed with specific substances, and then sterilized by introducing steam through the bioreactor mantle 72. This process can be done automatic using the control console/panel 125.

The embodiment presented in FIG. 12 can be used for the production of vaccines, drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

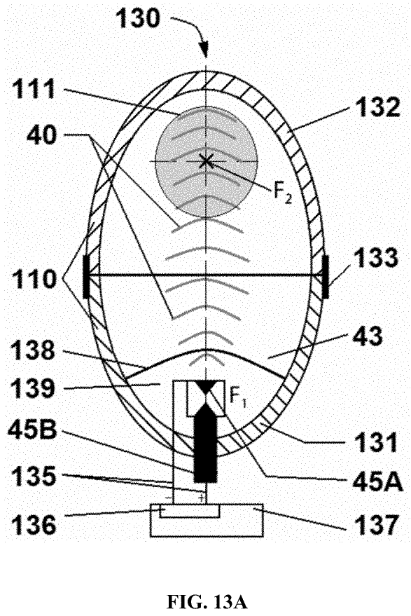

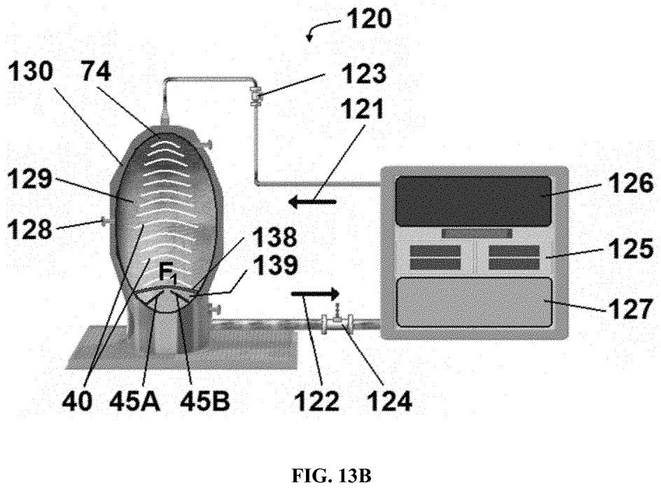

The embodiment from FIG. 13A shows a cross-section of a special bioreactor 130 that is made of the lower shell 131 and upper shell 132, which are connected together via the shells connecting ring 133. In this embodiment, the special bioreactor 130 has a deep full-ellipsoidal geometry that has a large major elliptical semiaxis “c” (as defined in FIG. 7) to create a full ellipsoidal reflector 110 with a large volume inside the special bioreactor 130 and to allow the proper shockwave or pressure wave stimulation and stirring of the whole volume of fluid-filled reflector cavity 43. For a deeper ellipsoidal geometry, the ratio in between the major elliptical semiaxis “c” and the minor elliptical semiaxis “b” (see FIG. 7) preferably may be larger than 1.9 (c/b≥1.9). When the spark-gap or laser electrohydraulic principle is used to generate shockwaves or pressure waves, the input high voltage discharge in F₁ (spark-gap 41 formed by the first electrode 45A and second electrode 45B) can be in between 14-35 kV. The high voltage for the first electrode 45A and the second electrode 45B is provided by the power supply 136 (included in control console/unit 137) via high voltage cable 135. The proposed construction with a full-ellipsoidal geometry of the special bioreactor 130 is using 80-90% of the ellipsoid (increased reflective area surface), compared with classic approach (semi-ellipsoidal reflector 42 presented for other embodiments) where only maximum 50% of the ellipsoid surface is used to focus the focused acoustic pressure shockwaves 40. The use of 80-90% of the ellipsoidal surface is done by combining a lower shell 131 with a distinctive upper shell 132, that are connected together via the shells connecting ring 133. This design provides a much higher efficiency in shockwave transmission, focusing towards the full ellipsoid focal volume 111, and a larger reflector cavity 43, which is filled with the cellular or bacterial culture 129 that it is in a fluid form that plays an important role in focusing and transmitting of the focused acoustic pressure shockwaves 40 or pressure waves throughout the fluid-filled reflector cavity 43. In order to not have detrimental consequences on the cellular or bacterial culture 129, the region where shockwaves or pressure waves are generated is separated from the rest of the reflector cavity 43 via a flexible and opaque separation wall 138 that transmits without loses the shockwaves or pressure waves into the cellular or bacterial culture 129. In this way a generator chamber 139 is created that is completely isolated from the cellular or bacterial culture 129. The opacity of the flexible and opaque separation wall 138 is necessary to protect the cellular or bacterial culture 129 from any flashes produced by the high voltage discharge in between the first electrode 45A and the second electrode 45B. The generator chamber 139 can be filled separately with degassed water or any fluid that facilitate the generation of the shockwaves or pressure waves via the spark-gap or laser electrohydraulic principle that relies on the formation and oscillation of a plasma bubble in the first focal point F₁ of the full ellipsoidal geometry of the special bioreactor 130. The generator chamber 139 is also necessary for the electromagnetic principle (using a cylindrical coil or a flat coil and lens) and for the piezoelectric principle (using piezo crystals or piezofibers) for the generation of shockwaves or pressure waves. If focused shockwaves are desired for the functionality of the special bioreactor 130, then for electromagnetic and piezoelectric principle, the geometry of the bioreactor for the generator chamber 139 preferably may be parabolic (as explained for the embodiments from FIGS. 19-22). This approach creates a combination geometry for the special bioreactor 130, with the lower shell 131 being parabolic and the upper shell 132 being ellipsoidal. If the focusing is not a requirement for the special bioreactor 130, then it can maintain the full ellipsoidal geometry also for the electromagnetic or piezoelectric principles for generating unfocused shockwaves or pressure waves. As presented for FIG. 11, in some cases both the lower shell 131 and the upper shell 132 can be parabolic in shape, which will form the full enclosure of the special bioreactor 130 by using two parabolic reflectors. That means that there will be two generator chambers 139 and two flexible and opaque separation walls 138, which will reduce the volume available for the cellular or bacterial culture 129 to approximative 70% to 75%. In all the embodiments presented for FIG. 13A the advantage is that the actual geometry of the special bioreactor 130 is used to create a large reflective area that allows the generation of shockwaves or pressure waves from one source or maximum two sources, to create a uniform and efficient stimulation and mixing of the cellular or bacterial culture 129. This is an advantage compared with the use of more than two separate shockwave/pressure wave devices 85A and 85B disposed around the cylindrical enclosure of the bioreactor 71, as presented in FIG. 12. Although, the use of multiple and separate shockwave/pressure wave devices 85A and 85B may offer some flexibility in parameters necessary for the optimum functionality of the bioreactor 71, the controlling of multiple and separate shockwave/pressure wave devices 85A and 85B it is more complex when compared to the embodiments from FIG. 13A. The control and the generation of optimum output parameters for all embodiments associated with FIG. 13A can be done much simpler and the actual overall construction is more compact for the special bioreactor 130. Furthermore, since the special bioreactor 130 is made of two independent shells (lower shell 131 and the upper shell 132) can significantly simplify the service and maintenance, by having a much easier access for cleaning, disinfection, and sterilization of the interior of the special bioreactor 130.

To increase the efficiency of special bioreactors 130, they can be part of an automatic bioreactor system 120, as presented in the embodiment from FIG. 13B. Knowing that the potency of shockwaves 40 increases with the larger reflective surface, it is clear that the special bioreactor 130 that practically is a full ellipsoidal reflector 110 (as seen in FIG. 13A) will be able to generate higher pressure gradients throughout its whole volume when compared to the shockwave/pressure wave devices 85A and 85B, presented in the embodiment from FIG. 12. When the spark-gap electrohydraulic principle is used to generate shockwaves or pressure waves, at the bottom of the special bioreactor 130 a plasma bubble is generated via the high voltage discharge in F₁ by the first electrode 45A and second electrode 45B. If a laser electrohydraulic system is used (similar to the one presented in FIG. 18), the two electrodes 45A and 45B are replaced by two incased lasers that must discharge confocal in the first focal point F₁ of the full ellipsoidal reflector 110 (as seen in FIG. 13A). The rest of practical aspects of functionality, applicability, and controlled directional movement are similar to those presented for the embodiment from FIG. 28. As seen before in FIG. 13A, in order to not have detrimental consequences on the cellular or bacterial culture 129, for all principles that are used to generate shockwaves or pressure waves (electrohydraulic, electromagnetic, or piezoelectric), the region where shockwaves or pressure waves are generated is separated from the rest of the reflector cavity 43 via a flexible and opaque separation wall 138, which creates the generator chamber 139 that is completely isolated from the cellular or bacterial culture 129. Furthermore, the automatic bioreactor system 120 uses a dedicated cellular or bacterial culture compartment 126 from where the cellular or bacterial culture introduction 121 is done at the top of the special bioreactor 130. The periodic introduction of the cellular or bacterial culture 129 inside the special bioreactor 130 is controlled via the control console/panel 125 that opens or closes the cellular and bacterial culture valve 123. During stimulation of the cellular or bacterial culture 129 that is mixed with the bioreactor nutrient broth 74, the original combined volume is used until the end of the process and in other situations new volumes of the cellular or bacterial culture 129 can be introduced periodically. Once a batch or certain volume of cellular or bacterial culture 129 is inside the bioreactor, then the shockwaves or pressure waves are actuated. The selection for actuation of the shockwaves or pressure waves and for their functional parameters can be done with specific artificial intelligence (A/I) algorithms that can adapt in time based on each situation and type and volume of cellular or bacterial culture 129. The functioning parameters for the special bioreactor 130, the cellular or bacterial culture 129 metabolism, and the monitoring of internal processes inside the bioreactor 71 via pH, culture temperature, dissolved oxygen tension, and media exchange measurements are done via probes or sensors 73, as presented in FIG. 7 (not shown specifically in FIG. 12). However, to access the cellular or bacterial culture 129, these probes or sensors 73 use probe or sensor ports 128 situated at the top or bottom or middle of the special bioreactor 130, as presented in FIG. 13B. The stimulation of the cellular or bacterial culture 129 is done for multiple days until the desired number of vaccine elements needed to produce the vaccine is accomplished. At that time point, the stimulation and mixing of the cellular or bacterial culture 129 via the shockwaves or pressure waves generated by the special bioreactor 130 is stopped and the vaccine component collection 122 is done from the bottom of the special bioreactor 130 and the storing of the solution with the vaccine components is done into the vaccine component solution compartment 127. The control console/panel 125 controls the vaccine component collection 122 via the opening or closing of the vaccine component valve 124. Regardless if the special bioreactor 130 process is continuous or discrete, it is necessary to have periodic cleaning, disinfection, sterilization and maintenance. For that the bioreactor 71 must emptied, washed with specific substances, and then sterilized. This process can be done automatic using the control console/panel 125.

All the embodiments associated with FIGS. 13A and 13B can be used for the production of vaccines, drugs, antibiotics, medications, mixture/cocktail of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

FIGS. 14A, 14B and 14C show cell/bacterial culture testing fixture 140 that is using shockwaves or pressure waves to enhance and expedite biological experimentation. This kind of fixture can be used at the beginning of the development process for vaccines, drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like. Such fixture can be used for example in the viral agent selection step 1000 presented in FIG. 8. The main goal of the cell/bacterial culture testing fixture 140 is to be able to generate and conduct the shockwaves or pressure waves gently towards the small volume cellular or bacterial culture 149 that is placed in a cell/bacterium culture well 148. The stimulation and stirring of the small volume cellular or bacterial culture 149 must be done without producing any cell or bacterium damage. The fixture can be easily adapted to different cell/bacterium culture well 148 diametral size, depending on the requirements of each biological experimentation. Also, the use of the open space 146 under the cell/bacterium culture well 148 is helping to avoid unnecessary reflection of the shockwaves or pressure waves from the bench table or support where the cell/bacterial culture testing fixture 140 sits during experimentation. In some cases that space can be filled with a foam material that attenuates and disperse shockwaves or pressure waves. Furthermore, the surface on which the cell/bacterial culture testing fixture 140 sits can have an open space in the continuation of the open space 146. The avoidance of the reflection back of active shockwaves or pressure waves towards the small volume cellular or bacterial culture 149 is needed to not create unnecessary shear forces in between the incoming and reflected shockwaves or pressure waves that can destroy the integrity of the cells or bacteria.

To accomplish its goals the cell/bacterial culture testing fixture 140 is using the shockwave/pressure wave device 85 that is connected via high voltage cable 135 to the power supply 136 that is included in control console/unit 137. When the spark-gap electrohydraulic principle is used to generate shockwaves or pressure waves (as presented in FIG. 14C), a plasma bubble is generated via the high voltage discharge in F₁ by the first electrode 45A and second electrode 45B. If a laser electrohydraulic system is used (similar to the one presented in FIG. 18), the two electrodes 45A and 45B are replaced by two incased lasers (45C and 45D) that must discharge confocal in the first focal point F₁ of the semi-ellipsoidal reflector 42 (as seen in FIG. 18). The semi-ellipsoidal reflector 42 will then reflect the shockwaves or pressure waves through the fluid-filled reflector cavity 43 towards the second focal point F₂ of the ellipsoidal geometry. The shockwave/pressure wave device 85 has the applicator/coupling membrane 44 sitting on top of the semi-ellipsoidal reflector 42, which creates the fluid-filled reflector cavity 43. Also, the applicator/coupling membrane 44 is the only element that gets in direct contact with the small volume cellular or bacterial culture 149. Based on its construction, the cell/bacterial culture testing fixture 140 is capable to precisely position the F₂ just inside the small volume cellular or bacterial culture 149 and half way through the height of the cell/bacterium culture well 148. This will assure a uniform stimulation and gentle stirring of the entire small volume cellular or bacterial culture 149 with focused shockwaves. To accomplish the correct aiming and positioning of the F₂ inside the cell/bacterium culture well 148, the location of the shockwave/pressure wave device 85 has to be well controlled by the cell/bacterial culture testing fixture 140. For that the shockwave/pressure wave device 85 is captured in between the fixture upper plate 141 and fixture lower plate 142. The cell/bacterium culture plate 147 is attached and fixed to the fixture lower plate 142. The fixture lower plate 142 has also four guiding columns 143 that have a portion threaded, which is identified as the column thread 143A, as seen in FIG. 14C. The cell/bacterium culture wells 148 containing the small volume cellular or bacterial culture 149 are dropped into the dedicated spaces into the cell/bacterium culture plate 147. Once a certain cell/bacterium culture well 148 is selected for the shockwave or pressure wave stimulation and stirring, the guiding cone 144 for the shockwave/pressure wave device 85 is secured in place into the dedicated space of the cell/bacterium culture plate 147 that leaves an exacting opening corresponding to the cell/bacterium culture well 148. Then the shockwave/pressure wave device 85 is dropped inside the guiding cone 144 until the frontal part of the applicator/coupling membrane 44 is in direct contact with the small volume cellular or bacterial culture 149 and the lateral conical surface of the applicator/coupling membrane 44 is in contact with the guiding cone 144. When the shockwave/pressure wave device 85 reached its optimal position, the membrane ring 145 of the shockwave/pressure wave device 85 it is in direct contact with the rim of the large opening of the guiding cone 144. Then the fixture upper plate 141 is dropped around the high voltage cable 135 of the shockwave/pressure wave device 85 that is not connected to the control console/unit 137 and its power supply 136. The upper plate 141 has four through holes corresponding to the four guiding columns 143 of the fixture lower plate 142 and also an upper plate central through hole 141A that corresponds to the shockwave/pressure wave device 85 dimension (see FIG. 14B). Once the upper plate 141 reaches the four guiding columns 143 and their column thread 143A, it preferably may be guided until its upper plate central through hole 141A gets in direct contact with the shockwave/pressure wave device 85, as seen in FIG. 14C. The upper plate 141 is then secured with four knob nuts 143B, which will guarantee the correct positioning of the shockwave/pressure wave device 85 relatively to the cell/bacterium culture well 148. Then the shockwave/pressure wave device 85 is connected to the control console/unit 137 and its power supply 136. At this point in time, the cell/bacterial culture testing fixture 140 is ready to stimulate and gentle mix the small volume cellular or bacterial culture 149. After the whole stimulation and mixing process is finished, by taking the steps in the reverse order the cell/bacterial culture testing fixture 140 is disassembled, the shockwave/pressure wave device 85 is removed, cleaned and disinfected, and the cell/bacterium culture well 148 is collected for further analysis or separation of different cellular or bacterial components.

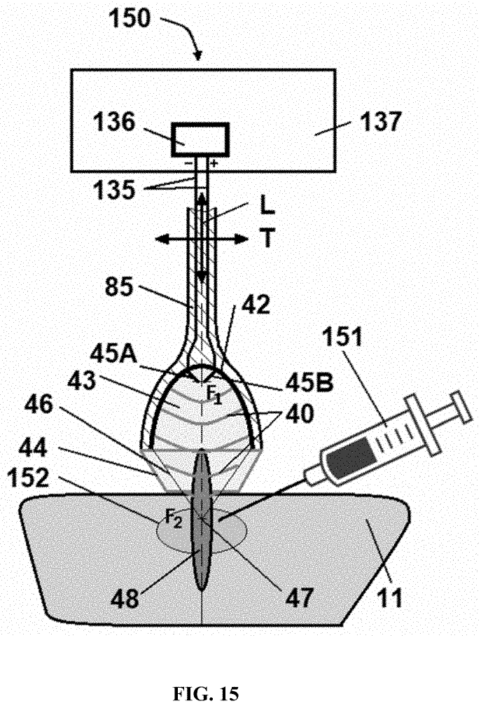

If the cell/bacterial culture testing fixture 140 is using an electrohydraulic shockwave/pressure wave device 85 that has a semi-ellipsoidal reflector 42, then the shockwaves produced will be focused (see FIGS. 15 and 18). In case F₂ is placed before or after the cell/bacterium culture well 148, then the shockwaves will be unfocused. When the cell/bacterial culture testing fixture 140 is using an electrohydraulic shockwave/pressure wave device 85 that has a parabolic reflector 51 (see FIG. 5), then only pressure waves (pseudo-planar pressure waves 50) will be produced, as presented in FIGS. 5 and 17. In the case when the shockwave/pressure wave device 85 is employing piezoelectric or electromagnetic principle (see FIGS. 19-22) and it has a parabolic reflector 51, then the shockwaves produced will be focused. If the fixture is using a piezoelectric or electromagnetic shockwave/pressure wave device 85 that has the semi-ellipsoidal reflector 42, then only pressure waves will be produced. If the combination semi-spherical and cylindrical reflector 160 from FIG. 16 is used for the shockwave/pressure wave device 85, in the cell/bacterium culture well 148 will be generated radial pressure wave 60, as seen in FIG. 6. Finally, the shockwave/pressure wave device 85 can be replaced with an ultrasound applicator 242 (similar to the one seen in FIG. 24B), which will produce low intensity ultrasound in the cell/bacterium culture well 148. All these options show the great flexibility that the cell/bacterial culture testing fixture 140 is offering for biological experimentation.

FIG. 15 presents a medication intake enhancement system 150 that is using the focused acoustic pressure shockwaves 40, which are generated via high voltage discharge produced in between first electrode 45A and the second electrode 45B (electrohydraulic principle using spark gap high voltage discharges) in a fluid present inside the reflector cavity 43. The high voltage for the first electrode 45A and the second electrode 45B is provided by the power supply 136 (included in control console/unit 137) via high voltage cable 135. The two electrodes 45A and 45B are positioned in the first focal point F₁ (forming the spark-gap 41, as presented in FIG. 4) of the semi-ellipsoidal reflector 42. During high voltage discharge a plasma bubble is generated that expands and collapses transforming the heat into kinetic energy in the form of acoustic pressure shockwaves that reflect on the semi-ellipsoidal reflector 42, and through shockwave focusing 46 are producing the focused acoustic pressure shockwaves 40, which are directed through the applicator/coupling membrane 44 towards the focal point 47 (F₂ of the ellipsoidal geometry) and overall to the focal volume 48 that overlaps with the targeted region from the human body 11 (or animal body) selected for vaccination with syringe 151, which is called injection site 152. To be able to properly/completely overlap with the focal volume 48 the entire injection site 152, the transversal (T) and longitudinal (L) motions of the shockwave/pressure wave device 85 are performed manually by the operator or by using semi-automatic or automatic means, if the targeted region is a larger one. The tissue or cellular stimulation can be done before or after vaccination. If the vaccination component is fragile, then the shockwaves preferably may be applied prior to injection, and the area must be marked to be able to do immediately the subsequent injection of the vaccine inside the stimulated area. In the case when shockwaves are applied immediately after the vaccine injection, the area that needs stimulation is much clear, since the puncture produced by the syringe 151 can be easily identified. In this case, the healthcare professional should position the shockwave/pressure device 85 in such way to be centered directly on top of the injection puncture. If the shockwave stimulation is done after vaccination, another advantage is given by the presence of the liquid vaccine inside the tissue, which will enhance the produced cavitation with benefic effects on permeabilization of the mammalian cells. Focused shockwaves have sufficient penetration to allow both superficial and deep cell stimulation for the rapid intake of the vaccine, regardless if they are applied before or after vaccination.

Although the exemplification of the use of medication intake enhancement system 150 was for vaccines as one example of a treatment agent, the embodiment presented in FIG. 15 can be also used for the delivery of other treatment agents intended for absorption by cells within a tissue, such as drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes focused shockwave systems (as the one presented in FIG. 15) one preferable treatment method to promote the precise transfer inside the targeted tissue or cells of the treating substance, drug, vaccine, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the focused shockwaves are known to increase blood circulation via blood vessels dilatation, to modulate inflammation, and to reduce or eliminate scarring, which means that the focused shockwaves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

In the embodiment from FIG. 16, the medication intake enhancement system 150 employs the shockwave/pressure wave device 85 that is using a combination semi-spherical and cylindrical reflector 160, which sends radial pressure waves 60 towards the human body 11 (or animal body) and injection site 152 selected for vaccination with syringe 151. The shockwave/pressure wave device 85 is producing the radial pressure waves 60 in the center F of semi-spherical reflector portion 160A of the combination semi-spherical and cylindrical reflector 160 that has in its upper part the cylindrical reflector portion 160B above the plane of the central point F and slightly tapered at the aperture (reflector's opening). The applicator/coupling membrane 44 sits at the aperture/opening of the combination semi-spherical and cylindrical reflector 160 and thus creating a fluid-filled reflector cavity 43. When the electrohydraulic principle is used to produce shockwaves or pressure waves, the “direct” radial pressure waves 60 are generated via the high voltage discharge between first electrode 45A and second electrode 45B and they travel from the center F of semi-spherical reflector portion 160A through the aperture of the shockwave/pressure wave device 85 and applicator/coupling membrane 44 directly to the injection site 152 without any reflection. The high voltage for the first electrode 45A and the second electrode 45B is provided by the power supply 136 (included in control console/unit 137) via high voltage cable 135. Due to the special construction of the combination semi-spherical and cylindrical reflector 160, the spheric waves/radial waves that are reaching the reflecting surface of the combination semi-spherical and cylindrical reflector 160 are reflected back towards the spherical center F or the longitudinal axis of the reflector. This avoids unnecessary reflected radial waves to be directed towards the injection site 152 that can interfere with the “direct” radial pressure waves 60. By their nature, the “direct” radial pressure waves 60 (exiting through the aperture of the combination semi-spherical and cylindrical reflector 160) are unfocused and thus they move through the radial waves pressure field 63 (seen in FIG. 6) and through the injection site 152 without being able to be concentrated/focused in a certain focal region, as seen before for the focused acoustic pressure shockwaves 40 (schematically shown in FIGS. 4, 10, 13A, and 15). Another way to create radial pressure waves 60 is given by ballistic devices that use pneumatics to push at high speeds a small cylindrical piece (bullet) against a plate that vibrates (due to the impact of the bullet) and thus creating/generating radial pressure waves 60. The ballistic devices were not specifically depicted in any of the figures of this invention, but can be used to generate radial pressure waves 60. To be able to properly and completely overlap and cover with the radial waves pressure field 63 (seen in FIG. 6) the entire injection site 152, the transversal (T) and longitudinal (L) motions of the applicator 97 are performed manually by the operator or by using semi-automatic or automatic means, if the targeted region is a larger one. As mentioned before for FIG. 15, the tissue or cellular stimulation can be done before or after vaccination, and all the details/comments related to this subject that were mentioned for FIG. 15 apply also to the embodiment from FIG. 16.

Although the exemplification of the use of medication intake enhancement system 150 was for vaccines, the embodiment presented in FIG. 16 can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes the radial pressure wave systems (as the one presented in FIG. 16) to promote the precise transfer inside the targeted tissue or cells of the treating substance, vaccine, drug, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the radial pressure waves are known to increase blood circulation via blood vessels dilatation, to modulate inflammation, and to reduce or eliminate scarring, which means that the radial pressure waves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

In the embodiment shown in FIG. 17, the medication intake enhancement system 150 employs a shockwave/pressure wave device 85 that is using a parabolic reflector 51, which sends pseudo-planar pressure waves 50 outside the applicator/coupling membrane 44 towards the human body 11 (or animal body) and through injection site 152 selected for vaccination with syringe 151. The parabolic reflector 51 has only a central point/focus point F (parabolic focal point) where radial pressure waves 60 are generated (via the high voltage discharge between first electrode 45A and second electrode 45B in the liquid present inside the reflector cavity 43). The radial pressure waves 60 propagate and reflect on the parabolic reflector 51 at different time points, which creates secondary pressure wave fronts (not shown on FIG. 17 to keep clarity), especially at the edge/aperture of the parabolic reflector 51. The combination of direct radial pressure waves 60 with the secondary pressure wave fronts creates pseudo-planar pressure waves 50 outside the applicator/coupling membrane 44, forming the pseudo-planar waves pressure field 55 (as seen in FIG. 5). In the embodiment from FIG. 17, the parabolic focal point F is inside the parabolic reflector 51 of the shockwave/pressure wave device 85 and this is why pseudo-planar pressure waves 50 are produced outside the applicator/coupling membrane 44 and pass through the human body 11 (or animal body) and injection site 152. This is different from the embodiments presented in FIGS. 19-22, where the parabolic focal point F is outside the parabolic reflector 51 of the shockwave/pressure wave device 85 and it is overlapping with the injection site 152. This is why the embodiments from FIGS. 19-22 produce focused acoustic pressure shockwaves 40 outside the applicator/coupling membrane 44.

By their nature, the pseudo-planar pressure waves 50 (exiting through the aperture of the parabolic reflector 51 and the applicator/coupling membrane 44) are unfocused and thus they move away from their point of origin F (parabolic focal point situated inside the parabolic reflector 51) without being able to be concentrated in a certain focal region, as seen for the focused acoustic pressure shockwaves 40 (see FIGS. 4, 10-15, and 18-22). The action of the pseudo-planar pressure waves 50 outside the applicator/coupling membrane 44 is controlled by the input energy, delivered via the cable 135 from the power supply 136 that is actuated by the control console/unit 137. To be able to properly stimulate the tissue and cells from the injection site 152, the pseudo-planar waves pressure field 55 (see FIG. 5), produced outside the applicator/coupling membrane 44 by the pseudo-planar pressure waves 50, needs to overlap and completely cover the injection site 152. To accomplish that the transversal (T) and longitudinal (L) motions of the shockwave/pressure wave device 85 are performed manually by the operator or by using semi-automatic or automatic means, if the targeted region is a larger one. As mentioned before for FIG. 15, the tissue or cellular stimulation can be done before or after vaccination, and all the details/comments related to this subject that were mentioned for FIG. 15 apply also to the embodiment from FIG. 17.

Although the exemplification of the use of medication intake enhancement system 150 was for vaccines, the embodiment presented in FIG. 17 can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes the pseudo-planar pressure wave systems (as the one presented in FIG. 17) to promote inside the targeted tissue or cells the precise transfer of the treating substance, vaccine, drug, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the pseudo-planar pressure waves are known to increase blood circulation via blood vessels dilatation, to modulate inflammation, and to reduce or eliminate scarring, which means that the pseudo-planar pressure waves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

In the embodiment presented in FIG. 18, the medication intake enhancement system 150 employs the shockwave/pressure wave device 85 that is using the focused acoustic pressure shockwaves 40, which are generated via one or multiple laser sources (electrohydraulic principle using one or multiple lasers sources). In this specific case the confocal laser beams produced by first incased laser 45C and the second incased laser 45D in a fluid present inside the reflector cavity 43 generate the acoustic pressure shockwaves 40, which are then focused via semi-ellipsoidal reflector 42 through applicator/coupling membrane 44 towards the focal point 47 (F₂ of the ellipsoidal geometry) from inside the human body 11 (or animal body) and to the focal volume 48 that overlaps with the injection site 152 selected for vaccination with syringe 151. The high voltage for the first incased laser 45C and the second incased laser 45D is provided by the power source 136 (included in control/console unit 137) via high voltage cable 135. The two laser sources are positioned in such way to intersect their beams in the first focal point F₁ of the semi-ellipsoidal reflector 42 in order to produce a plasma bubble that expands and collapses transforming the heat into kinetic energy in the form of acoustic pressure shockwaves that reflect on the semi-ellipsoidal reflector 42, and through shockwave focusing 46 are producing the focused acoustic pressure shockwaves 40, which are directed towards the focal point 47 (F₂ of the ellipsoidal geometry) and to the focal volume 48 that overlaps with the injection site 152. FIG. 18 includes a means of monitoring the system performance by measuring the reaction temperature of the plasma bubble collapse using a method of optical fiber thermometry. An optical fiber tube assembly 180 extends into the F₁ region of the semi-ellipsoidal reflector 42. The optical fiber tube assembly 180 transmits (via optical fiber 181) specific spectral frequencies created from the sonoluminescence of the plasma bubble reaction in the fluid present inside the reflector cavity 43 to the spectral analyzer 182. The loop is closed via feedback cable 183 that connects the spectral analyzer 182 with the power supply 136. Basically, the spectral analysis provided by the spectral analyzer 182 is used to adjust accordingly the power generated by the power supply 136, to ensure a proper laser discharge for the incased lasers 45C and 45D. To be able to properly overlap and cover with the focal volume 48 the entire injection site 152, the transversal (T) and longitudinal (L) motions of the applicator 97 are performed manually by the operator or by using semi-automatic or automatic means, if the targeted region is a larger one. As mentioned before for FIG. 15, the tissue or cellular stimulation can be done before or after vaccination, and all the details/comments related to this subject that were mentioned for FIG. 15 apply also to the embodiment from FIG. 18.

Although the exemplification of the use of medication intake enhancement system 150 was for vaccines, the embodiment presented in FIG. 18 can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes the focused shockwave systems (as the one presented in FIG. 18) to promote the precise transfer inside the targeted tissue or cells of the treating substance, drug, vaccine, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the focused shockwaves are known to increase blood circulation via blood vessels dilatation, to modulate inflammation, and to reduce or eliminate scarring, which means that the focused shockwaves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

In FIGS. 15 and 18, where the electrohydraulic principle is used to produce focused acoustic pressure shockwaves 40, if the semi-ellipsoidal reflector 42 is replaced with a parabolic reflector 51 (see FIG. 5) that has its parabolic focal point 53 (F) in the same position as the first focal point (F₁) of the semi-ellipsoidal reflector 42, then the shockwave/pressure wave device 85 will produce pseudo-planar pressure waves 50, similar to those from the embodiment presented in FIG. 17.

In the embodiment from FIG. 19, the medication intake enhancement system 150 employs the shockwave/pressure wave device 85 that is using the focused acoustic pressure shockwaves 40, which are generated via electromagnetic cylindrical coil and tube assembly 4511 (electromagnetic principle using a cylindrical coil). In this case, an electromagnetic cylindrical coil is excited by a short electrical pulse provided by the power supply 136 (included in control console/unit 137) via high voltage cable 135. The cylindrical coil is inside of a tube (thus creating an electromagnetic cylindrical coil and tube assembly 4511). When the electromagnetic cylindrical coil is excited by a short electrical pulse provided by the power supply 136 via high voltage cable 135, the cylindrical coil experiences a repulsive force and this is used to generate a cylindrical acoustic pressure wave inside the fluid-filled reflector cavity 43 that is reflected on the parabolic reflector 51, thus creating focused acoustic pressure shockwaves 40. The parabolic reflector 51 produce the shockwave focusing 46 through applicator/coupling membrane 44 towards the parabolic focal point 53 (F) from inside the human body 11 (or animal body) and to the focal volume 48 that overlaps with the injection site 152 selected for vaccination with syringe 151.

Conversely, in another embodiment for the medication intake enhancement system 150 from FIG. 19, the parabolic reflector 51 can be replaced by a semi-ellipsoidal reflector 42 to create unfocused pressure waves that generate a pressure field outside the applicator/coupling membrane 44 of the semi-ellipsoidal reflector 42, pressure field that needs to overlap with the injection site 152.

To be able to stimulate cellular or tissue permeability, the shockwave/pressure wave device 85 needs to completely cover the injection site 152 with the focal volume 48 (for focused acoustic pressure shockwaves 40) or with the pressure field produced outside the applicator/coupling membrane 44 by unfocused pressure waves, when the parabolic reflector 51 is replaced by a semi-ellipsoidal reflector 42. To accomplish that the transversal (T) and longitudinal (L) motions of the shockwave/pressure wave device 85 are performed manually by the operator or by using semi-automatic or automatic means, if the targeted region is a larger one. As mentioned before for FIG. 15, the tissue or cellular stimulation can be done before or after vaccination, and all the details/comments related to this subject that were mentioned for FIG. 15 apply also to the embodiment from FIG. 19.

Although the exemplification of the use of medication intake enhancement system 150 was for vaccines, the embodiment presented in FIG. 19 can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes the focused shockwave or unfocused pressure wave systems (as the one presented in FIG. 19) to promote the precise transfer inside the targeted tissue or cells of the treating substance, drug, vaccine, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the focused shockwaves or unfocused pressure waves are known to increase blood circulation via blood vessels dilatation, to modulate inflammation, and to reduce or eliminate scarring, which means that the focused shockwaves or unfocused pressure waves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

In the embodiment from FIG. 20, the medication intake enhancement system 150 employs the shockwave/pressure wave device 85 that is using the focused acoustic pressure shockwaves 40, which are generated via electromagnetic flat coil and plate assembly 45G and an acoustic lens 200 (electromagnetic principle using a flat coil and an acoustic lens). In this case, an electromagnetic flat coil is placed in close proximity to a metal plate that acts as an acoustic source and thus the electromagnetic flat coil and plate assembly 45G presented in FIG. 20 is created. When the electromagnetic flat coil is excited by a short electrical pulse provided by the power supply 136 (included in control console/unit 137) via high voltage cable 135, the plate experiences a repulsive force and this is used to generate an acoustic pressure wave. Due to the fact that the metal plate is flat, the resulting acoustic pressure wave is a planar acoustic pressure wave (not shown in FIG. 20) that is moving in the fluid-filled cavity 201 towards the acoustic lens 200, which is focusing the planar wave (shockwave focusing 46) and thus creating the focused acoustic pressure shockwaves 40. The focusing effect of the acoustic lens 200 is given by its shape, which as presented in FIG. 20 is a portion of a parabolic surface. This is why the acoustic lens 200 is used in tandem with a parabolic reflector 51 that can help with the focusing of the produced focused acoustic pressure shockwaves 40 through applicator/coupling membrane 44 towards the parabolic focal point 53 (F) and to the focal volume 48 that overlaps with the injection site 152 selected for vaccination with syringe 151.

Conversely, in another embodiment the acoustic lens 200 can be a portion of an ellipsoidal surface and in combination with a semi-ellipsoidal reflector 42 can create unfocused pressure waves that can generate a pressure field outside the applicator/coupling membrane 44 of the semi-ellipsoidal reflector 42, pressure field that needs to overlap with the injection site 152.

To be able to stimulate cellular or tissue permeability, the shockwave/pressure wave device 85 needs to completely cover the injection site 152 with the focal volume 48 (for focused acoustic pressure shockwaves 40) or with the pressure field produced outside the applicator/coupling membrane 44 by unfocused pressure waves, when the parabolic reflector 51 is replaced by a semi-ellipsoidal reflector 42. To accomplish that the transversal (T) and longitudinal (L) motions of the shockwave/pressure wave device 85 are performed manually by the operator or by using semi-automatic or automatic means, if the targeted region is a larger one. As mentioned before for FIG. 15, the tissue or cellular stimulation can be done before or after vaccination, and all the details/comments related to this subject that were mentioned for FIG. 15 apply also to the embodiment from FIG. 20.

Although the exemplification of the use of medication intake enhancement system 150 was for vaccines, the embodiment presented in FIG. 20 can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes the focused shockwave or unfocused pressure wave systems (as the one presented in FIG. 20) to promote the precise transfer inside the targeted tissue or cells of the treating substance, drug, vaccine, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the focused shockwaves or unfocused pressure waves are known to increase blood circulation via blood vessels dilatation, to modulate inflammation, and to reduce or eliminate scarring, which means that the focused shockwaves or unfocused pressure waves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

In the embodiment from FIG. 21, the medication intake enhancement system 150 employs the shockwave/pressure wave device 85 that is using the focused acoustic pressure shockwaves 40, which are generated via piezo crystals/piezo ceramics 45E (piezoelectric principle using piezo crystals/piezo ceramics). In this case, the internal generation of a mechanical strain resulting from an applied electrical field to the piezo crystals/piezo ceramics 45E, which are uniformly placed on a parabolic reflector 51, generate in a fluid present inside the reflector cavity 43 the focused acoustic pressure shockwaves 40. The parabolic reflector 51 produces the shockwave focusing 46 through applicator/coupling membrane 44 towards its focal point F (parabolic focal point 53) from inside the human body 11 (or animal body) and to the focal volume 48 that overlaps with the injection site 152 selected for vaccination with syringe 151. To accomplish the shockwave focusing 46, all the surface of the parabolic reflector 51 is covered by the piezo crystals/piezo ceramics 45E. The electrical field for the piezo crystals/piezo ceramics 45E is provided by the power supply 136 (included in control console/unit 137) via high voltage cable 135.

Relatively similar effects can be accomplished when the piezo crystals/piezo ceramics 45E are used together with the semi-ellipsoidal reflector 42. In this case, since the pressure waves are originating from the surface of the semi-ellipsoidal reflector 42 and not from the focal point F₁ of the ellipsoidal geometry, the produced pressure waves fall in the category of unfocused pressure waves and not shockwaves. The unfocused pressure waves can generate a pressure field outside the applicator/coupling membrane 44 of the semi-ellipsoidal reflector 42, pressure field that needs to overlap with the injection site 152.

To be able to stimulate cellular or tissue permeability, the shockwave/pressure wave device 85 needs to completely cover the injection site 152 with the focal volume 48 (for focused acoustic pressure shockwaves 40) or with the pressure field produced outside the applicator/coupling membrane 44 by unfocused pressure waves, when the parabolic reflector 51 is replaced by a semi-ellipsoidal reflector 42. To accomplish that the transversal (T) and longitudinal (L) motions of the shockwave/pressure wave device 85 are performed manually by the operator or by using semi-automatic or automatic means, if the targeted region is a larger one. As mentioned before for FIG. 15, the tissue or cellular stimulation can be done before or after vaccination, and all the details/comments related to this subject that were mentioned for FIG. 15 apply also to the embodiment from FIG. 21.

Although the exemplification of the use of medication intake enhancement system 150 was for vaccines, the embodiment presented in FIG. 21 can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes the focused shockwave or unfocused pressure wave systems (as the one presented in FIG. 21) to promote the precise transfer inside the targeted tissue or cells of the treating substance, drug, vaccine, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the focused shockwaves or unfocused pressure waves are known to increase blood circulation via blood vessels dilatation, to modulate inflammation, and to reduce or eliminate scarring, which means that the focused shockwaves or unfocused pressure waves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

Due to the parallelepiped or cylindrical geometry of the piezo crystals/piezo ceramics 45E, they may not fit very well to the parabolic reflector 51 surface, which can create problems with focusing towards the parabolic focal point 53 (F), especially in situations where deep penetrations are needed, since these geometries will require a sharp vertex of the parabola with smaller radiuses that are difficult to cover with parallelepiped or cylindrical piezo crystals/piezo ceramics 45E. To overcome this issue, the piezo crystals/piezo ceramics 45E can be replaced by piezo fibers in the construction of a shockwave/pressure wave device 85, as presented in FIG. 22. The piezo fibers can be integrated in a composite material with their longitudinal axis perpendicular to a solid surface as the parabolic reflector 51, thus forming a piezo fiber layer 45F capable of producing focused acoustic pressure shockwaves 40. The advantage of the piezo fiber layer 45F when compared to the piezo crystals/piezo ceramics 45E is that the smaller dimension and cylindrical geometry (hair-like geometry) of the piezo fibers allows them to fit significantly better to the parabolic or ellipsoidal reflector geometries. Furthermore, the electric contacting of the piezo fibers may be realized by a common electrically conductive layer according to the interconnection requirements. Hence, the complex interconnection of a multitude of piezo crystals/piezo ceramics 45E (as presented in FIG. 21) is no longer required. When an electrical field is provided by the power supply 136 (included in control console/unit 137) via high voltage cable 135 to the piezo fiber layer 45F, the piezo electric fibers will stretch in unison mainly in their lengthwise direction, which will create focused acoustic pressure shockwaves 40 from the surface of the parabolic reflector 51 that is producing shockwave focusing 46 through applicator/coupling membrane 44 towards the parabolic focal point 53 (F) from inside the human body 11 (or animal body) and overall, to the focal volume 48 that overlaps with the injection site 152 selected for vaccination with syringe 151.

Relatively similar effects can be accomplished when the piezo fiber layer 45F is used together with a semi-ellipsoidal reflector 42, but in this case since the pressure waves are originating from the surface of the semi-ellipsoidal reflector 42 and not from the focal point F₁ of the ellipsoidal geometry, the produced pressure waves fall in the category of unfocused waves and not shockwaves. The unfocused pressure waves can generate a pressure field outside the applicator/coupling membrane 44 of the semi-ellipsoidal reflector 42, pressure field that needs to overlap with the injection site 152.

To be able to stimulate cellular or tissue permeability, the shockwave/pressure wave device 85 needs to completely cover the injection site 152 with the focal volume 48 (for focused acoustic pressure shockwaves 40) or with the pressure field produced outside the applicator/coupling membrane 44 by unfocused pressure waves, when the parabolic reflector 51 is replaced by a semi-ellipsoidal reflector 42. To accomplish that the transversal (T) and longitudinal (L) motions of the shockwave/pressure wave device 85 are performed manually by the operator or by using semi-automatic or automatic means, if the targeted region is a larger one. As mentioned before for FIG. 15, the tissue or cellular stimulation can be done before or after vaccination, and all the details/comments related to this subject that were mentioned for FIG. 15 apply also to the embodiment from FIG. 22. Further, the pressure waves are applied with corresponding parameters as described herein to avoid killing or destroying a majority of targeted tissue, i.e. a majority of cells in the targeted tissue remaining viable and intact, where the focal volume 48 or pressure field intersects the targeted tissue.

Although the exemplification of the use of medication intake enhancement system 150 was for vaccines, the embodiment presented in FIG. 22 can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes the focused shockwave or unfocused pressure wave systems (as the one presented in FIG. 22) to promote the precise transfer inside the targeted tissue or cells of the treating substance, drug, vaccine, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the focused shockwaves or unfocused pressure waves are known to increase blood circulation via blood vessels dilatation, to modulate inflammation, and to reduce or eliminate scarring, which means that the focused shockwaves or unfocused pressure waves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

The embodiment from FIG. 23 is a medication intake piezoelectric system 230 that is capable to easily generate planar pressure waves 234 by using piezo crystals/piezo ceramics or piezo fibers layer 235. These shockwave/pressure wave devices 85 can be used to generate planar pressure waves 234 and direct them inside the human body 11 (or animal body) and towards the injection site 152 selected for vaccination with syringe 151. In order to get the shockwave/pressure wave devices 85 in contact with the surface/skin of the human body 11 (or animal body), the shockwave/pressure wave devices 85 is moved via transversal (T) and longitudinal (L) motions performed manually by the operator or by using semi-automatic or automatic means, if the targeted region is a larger one. By applying an electrical field to the piezo crystals/piezo ceramics or piezo fibers layer 235 that is placed uniformly on the central core 271 (can be cylindrical, square, hexagonal, octagonal or decagonal, etc.), a mechanical strain is resulting that produces planar pressure waves 234 inside the fluid-filled cavity 43 formed in between the upper cover 232 and the lateral semi-cylindrical coupling membrane 233. The electrical field for the piezo crystals/piezo ceramics or piezo fibers layer 235 is provided via high voltage cable 135 by the power supply 136, which is included in control console/unit 137. The shockwave/pressure wave devices 85 have an upper cover 232 that is the support for the lateral semi-cylindrical coupling membrane 233 and also helps with the proper orientation of the shockwave/pressure wave devices 85 relatively to the human body 11 (or animal body), to correctly direct the planar pressure waves 234 through the human body 11 (or animal body) and injection site 152. If the design/construction of the piezo crystals/piezo ceramics or piezo fibers layer 235 uses independent piezo crystals/piezo ceramics, then individual or multiple piezo crystals/piezo ceramics can be activated concomitantly or sequentially, which can tailor the delivery of the planar pressure waves 234 based on explicit needs of the inducement of augmented cellular or tissue permeability process. When piezofibers are used in the construction of the piezo crystals/piezo ceramics or piezo fibers layer 235, their activation can be done only concomitantly, since a common electrically conductive layer is used for such systems. As mentioned before for FIG. 15, the tissue or cellular stimulation can be done before or after vaccination, and all the details/comments related to this subject that were mentioned for FIG. 15 apply also to the embodiment from FIG. 23.

Although the exemplification of the use of medication intake piezoelectric system 230 was for vaccines, the embodiment presented in FIG. 23 can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate deep inside the human and animal body, and the inducement of augmented cellular permeability makes the planar pressure wave systems (as the one presented in FIG. 23) to promote the precise transfer inside the targeted tissue or cells of the treating substance, drug, vaccine, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the planar pressure waves are known to increase blood circulation via blood vessels dilatation, to modulate inflammation, and to reduce or eliminate scarring, which means that the planar pressure waves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

FIG. 24A shows the features characteristic of the ultrasound pressure waves, which were discussed in detail previously when all type of shockwaves or pressure waves were compared at the beginning at this invention. FIG. 24B presents an embodiment that uses low frequency ultrasound waves 241 to produce cellular activation and stimulation for vaccine intake via a medication intake ultrasound system 240.

Inside the ultrasound applicator 242 and also inside the applicator/coupling membrane 44, there is a central metal acoustic horn 244 and the ultrasound-generating piezo crystal/piezo ceramic 243 that are is to produce the ultrasound waves 241. The ultrasound-generating piezo crystal/piezo ceramic 243 converts and transfers the input electrical power received via power cable 135 from the power supply 136 (included in the control console/unit 137) into vibrational mechanical (ultrasonic) energy that will be delivered via the ultrasound transmission fluid 245 and the applicator/coupling membrane 44 to the human body 11 (or animal body) and injection site 152 selected for vaccination with syringe 151. The acoustic horn 244 is used to amplify the excitation of the ultrasound-generating piezo crystal/piezo ceramic 243 to increase the ultrasound amplitude 248A (see FIG. 24A) and thus produce a more robust and energetic ultrasound waves 241 that exits the applicator/coupling membrane 44 along the central longitudinal axis of the ultrasound applicator 242.

The ultrasound-generating piezo crystal/piezo ceramic 243 has the frontal surface radial to be able to radiate the main ultrasound waves 241 in a radial/spherical manner. In order to get the ultrasound applicator 242 in contact with the surface/skin of the human body 11 (or animal body), the ultrasound applicator 242 is moved via transversal (T) and longitudinal (L) motions performed manually by the operator or by using semi-automatic or automatic means, if the targeted region is a larger one. As mentioned before for FIG. 15, the tissue or cellular stimulation can be done before or after vaccination, and all the details/comments related to this subject that were mentioned for FIG. 15 apply also to the embodiment from FIG. 24B.

Although the exemplification of the use of medication intake ultrasound system 240 was for vaccines, the embodiment presented in FIG. 24B can be also used for the delivery of drugs, antibiotics, medications, mixtures/cocktails of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The extracorporeal approach, the possibility to penetrate inside the human and animal body, and the inducement of augmented cellular permeability makes the low intensity ultrasound systems (as the one presented in FIG. 24B) to promote the precise transfer inside the targeted tissue or cells of the treating substance, drug, vaccine, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the low intensity ultrasound waves are known to increase blood circulation via blood vessels dilatation, to modulate inflammation, and to reduce or eliminate scarring, which means that the low intensity ultrasound waves can also be used to prepare the area before the vaccines or drugs or antibiotics or medications or gene medicines or protein materials (e.g., antibodies, anti-CRISPR proteins, etc.) or cellular materials (stem cells, immune cells, etc.) delivery.

In FIGS. 25A and 25B are presented embodiments that use a special syringe system 250 that can be applied to deliver a liquid medical substance 254 via an injection with the pressure wave syringe 251 and concomitantly being capable to activate with pressure waves 252 the targeted cells, tissues, or organs, to facilitate a fast intake of the liquid medical substance 254. Initially, the liquid medical substance 254 is aspirated from a dedicated container (not shown in FIGS. 25A and 25B) in the syringe medical substance chamber 251D. The liquid medical substance 254 fills in the space in between the syringe plunger 251A, the syringe medical substance chamber 251D towards the syringe funnel 252B and down to the end of the syringe needle 251C. The syringe plunger 251A has a special shape at the proximal end in the form of a pressure wave reflector 253, which is similar to any of the reflectors presented in other embodiments of these inventions (semi-ellipsoidal reflector 42, parabolic reflector 51, combination semi-spherical and conical reflector 61, combination semi-spherical and cylindrical reflector 160, or any combination of them). The pressure waves 252 can be generated via electrohydraulic (spark-gap or laser), electromagnetic (cylindrical coil or flat coil and lens), or piezoelectric (piezo-crystals/piezo-ceramics or piezo fibers) principles. The power used to generate the pressure waves 252 can come from an internal power source made of batteries or from an external power source that is using a cable and normal plug-in to wall outlets (not shown in FIGS. 25A and 25B for simplicity). The liquid medical substance 254 is capable to conduct the pressure waves 252 from the pressure wave reflector 253 (where they are generated) through the liquid medical substance 254, through the body of the pressure wave syringe 251 in the form of pressure waves from syringe body 252A, and then through syringe funnel 251B in the form of pressure waves from syringe funnel 252B. The syringe funnel 251B is in direct contact with the human body 11 (or animal body). The human body 11 (or animal body) has similar acoustic impedance as the liquid medical substance 254, which allows the transmission of the pressure waves from syringe funnel 252B without any loses into the human body 11 (or animal body) in the form of pressure waves inside the body 252C. The pressure waves inside the body 252C will be able to stimulate the cells and tissue for a rapid intake of the liquid medical substance 254 after injection from the pressure wave syringe 251.

As presented in FIG. 25A, the special syringe system 250 is first loaded with the liquid medical substance 254 from a special container (not shown in FIG. 25A) via the syringe needle 251C, by pulling up the syringe plunger 251A. Once the syringe medical substance chamber 251D is filled completely with the liquid medical substance 254 and no air is trapped inside (as it happens for any injection that uses a syringe and a liquid medical substance), then the pressure wave syringe 251 has its syringe needle 251C penetrating the skin into the desired region for the injection of the liquid medical substance 254. In this way the syringe funnel 252B is put in direct contact with the human body 11 (or animal body) via a small quantity of ultrasound gel that was already pre-applied before the syringe needle 251C was inserted inside the human body 11 (or animal body). At this point in time, the generation of the pressure waves 252 is started when the actuation of the pressure wave actuation button 255 is pressed. Due to pressure wave reflector 253, the pressure waves 252 start to propagate towards the human body 11 (or animal body) in the form of pressure waves from syringe body 252A, pressure waves from syringe funnel 252B, and pressure waves inside the body 252C. The pressure waves from syringe body 252A will reduce their dimensional area of action when they get to the syringe funnel 251B, due to the smaller dimension of the syringe funnel 251B. This is why some of the pressure waves from syringe body 252A will reflect from the conical area of the syringe backwards toward the pressure wave reflector 253. To avoid any interference with the subsequent pressure waves, the frequency of the pressure waves preferably may be 2 to 4 pressure waves per second (2-4 Hz). For a proper stimulation of the cells or tissues, the special syringe system 250 preferably may be able to produce a flux density of 0.010 to 0.300 mJ/mm², a total number of pressure waves in between 50 to 200, and the compressive pressures generated by the pressure waves preferably may vary in between 5 MPa to 40 MPa. The pressure waves inside the body 252C have enough spread to stimulate the proper number of cells or tissue for a rapid intake of the liquid medical substance 254. The dimension of the syringe funnel 252B can be done with a larger diameter or in some embodiments the syringe funnel 252B can be totally eliminated, which will allow the direct transmission of the pressure waves from syringe body 252A inside the human body 11 (or animal body). This approach can be applied when the area of stimulation needs to be larger. In another embodiment the syringe needle 251C can be taken off during stimulation, which allows the special syringe system 250 to be moved with a transversal move L (shown in other figures of these inventions), to cover a significant larger area of stimulation. After stimulation a significant larger area with the special syringe system 250, the syringe needle 251C is put back on the pressure wave syringe 251 and the injection of the liquid medical substance 254 can be done subsequently in the previously stimulated region with pressure waves inside the body 252C.

Going back to FIG. 25A, the progress of the pressure wave stimulation can be followed-up on the display 257 and the pressure waves 252, 252A, and 252C can be stopped any time using the pressure wave stop button 256. During pressure wave stimulation (with the syringe needle 251C in place) the operator can simultaneously inject the liquid medical substance 254 by pushing down the syringe plunger 251A via the syringe plunger movement 251E. Due to the fact that pressure wave reflector 253 moves also down with the syringe plunger 251A during the syringe plunger movement 251E, the shock waves 252, 252A, and 252C can be continuously produced in the liquid medical substance 254 for all the time during injection. Furthermore, as shown in FIG. 25B, even when the syringe plunger 251A and its associated pressure wave reflector 253 reach their final position after injecting the majority of the liquid medical substance 254, the pressure waves 252, 252A, and 252C can still be produced in the small quantity of liquid medical substance 254 left inside the pressure wave reflector 253 and the syringe funnel 252B. This means the stimulation can be continued for a short period of time after the injection of the liquid medical substance 254 into the injection site 152. The operator can always monitor the delivery of the pressure waves using the display 257 and can at any time start the pressure waves 252 using the pressure wave actuation button 255 or stop the pressure waves 252 using the using the pressure wave stop button 256.

In the embodiment from FIGS. 25A and 25B, the liquid medical substance 254 can be a vaccine, drug, antibiotic, medication, mixture/cocktail of multiple active ingredients, gene medicine, stem cell, stem cell cocktail, DNA or RNA/mRNA genetic material, genetic modified material, immune cell cocktail, base editing mean, liquid cocktail of liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like.

The advantage of the embodiments of these inventions described in FIGS. 25A and 25B is that with the special syringe system 250 the stimulation and injection is accomplished with only one system, which makes everything easier for the operator. Furthermore, the extracorporeal approach, the possibility to penetrate deep inside the human and animal body, and the inducement of augmented cellular permeability makes the pressure waves generated by the special syringe system 250 (as the one presented in FIGS. 25A and 25B) to promote inside the targeted tissue or cells the precise transfer of the treating substance, vaccine, drug, medication, antibiotic, gene medicine, protein material (antibodies, anti-CRISPR proteins, etc.), or cellular material (stem cells, immune cells, etc.) and facilitate proper tissue or cellular activation and stimulation in desired/targeted areas of the human or animal body. Furthermore, the pressure waves are known to increase blood circulation via blood vessels dilatation, to modulate inflammation, and to reduce or eliminate scarring, which means that the specifically modulated pressure waves produced by the special syringe system 250 can be used to properly prepare the area for the vaccines or drugs or antibiotics or medications or gene medicines or protein materials (e.g., antibodies, anti-CRISPR proteins, etc.) or cellular materials (stem cells, immune cells, etc.) delivery.

The shockwave or pressure wave or ultrasound system can be independent, portable or can be part of the syringe used for vaccination. The actual shockwave or pressure wave or ultrasound system can be integral part of the syringe or can be an attachable component to the syringe and thus can service more than one syringe, which is more economically. In this case the shockwave system can be operated via plug in or batteries. Shockwaves will be less powerful, but with enough energy to open the cells vesicles via mechanotransduction.

The construction of the medication intake special syringe system 250 used to stimulate the tissue or cellular intake of liquid drugs, vaccines, medications, genetic medicines, monoclonal antibodies, should have a simple construction. As mentioned before, it preferably may be an optimum shockwave or pressure wave dosage (frequency, number of or pressure waves and energy setting) or ultrasound setting (energy, frequency, and duration) that is the same and independent of the type of vaccine. The same reasoning can be applied for the delivery inside the cells of different liquid drugs, antibiotics, medications, mixture/cocktail of multiple active ingredients, gene medicines, stem cells, stem cell cocktails, DNA or RNA/mRNA genetic materials, genetic modified materials, immune cells, base editing means, liposomes or lipid nano or micro-particles or any other artificially or natural envelope incorporating active substances, drugs, antibiotics, medications, vaccine materials, nano-robots, nano-particles, genetic materials, genetic modified materials, specific proteins, antibodies, stem cells, and the like. This is why the control of medication intake special syringe system 250 do not need complicated software for the user interface in order to change the required parameters (should not have too many options to change parameters). However, alternatively the control console preferably may be capable of using artificial intelligence to generate an optimum set-up algorithm for each liquid medical substance, and thus program the treatment parameters automatically without the need of the user input. That can be accomplished by scanning a code associated with the medical substance that will choose the right algorithm for stimulate the vaccine after injection.

All the membranes from the embodiments of these inventions are made of a soft plastic material that are soft to the skin when in contact with it. Also, the soft plastic material of the membranes is chosen from materials that have acoustic properties very close to the fluid used inside the shockwave/pressure wave devices 85 or ultrasound applicator 242 to not impede with the propagation of focused acoustic pressure shockwaves 40 or pressure waves (acoustic planar pressure waves 234 or pseudo-planar pressure waves 50 or radial pressure waves 60) and low-frequency ultrasound waves 241.

When RNA or genetic material for vaccines is produced via bacteria, the shockwaves or pressure waves or ultrasound waves can be used to break the bacterial membrane and facilitate the extraction of the genetic material fragments.

Some treatments may require multiple injections, in the same region or in adjacent regions of the human body 11 (or animal body), with one or multiple liquid medical substances. The embodiments presented in these inventions can be used for such situations, since the devices presented in FIGS. 15-25B are capable to perform preparation and stimulation of the targeted cells, tissues, or organs and administer multiple doses subsequently or concomitantly.

In conclusion, the embodiments that use shockwaves or pressure waves or ultrasound waves, as presented in these inventions can be used for the following:
to stimulate small cell cultures or bacterial cultures in order to produce different components of the vaccines, discrete elements as DNA, nude mRNA, or proteins, or even live viruses, or monoclonal antibodies (laboratory-made proteins that mimic the immune system's ability to fight off harmful pathogens such as viruses), or gene medicines (see embodiments from FIGS. 8, 12, 13A-14C and the shockwave or pressure wave or ultrasound devices presented in FIGS. 15-24B);
to facilitate in combination with heat different chemical reactions and to maintain the cell or bacterial cultures at certain desired temperatures (see embodiments from FIGS. 8, 12, 13A-13B and the shockwave or pressure wave or ultrasound devices presented in FIGS. 15-24B);
to expedite enzymatic processes and reactions by using continuous agitation of the cell or bacterial culture and increase of enzymatic activity, which is produced by the acoustic streaming of the compressive phase of the shockwaves or pressure waves or ultrasound or due to acoustic microstreaming produced by the collapse of cavitational bubbles that were generated by the negative pressures from the tensile phase of the shockwaves or pressure waves or ultrasound (see embodiments from FIGS. 8, 12, 13A-14C and the shockwave or pressure wave or ultrasound devices presented in FIGS. 15-24B);
to separate the antibodies from the blood plasma collected from patients with immunity and stimulate the multiplication of such antibodies due to the gentle mechanical stimulation produced by the shockwaves or pressure waves or ultrasound in the presence or absence of viral components, thus reducing the necessity for periodic blood donations from such patients with immunity (see embodiments from FIGS. 8, 12, 13A-14C and the shockwave or pressure wave or ultrasound devices presented in FIGS. 15-24B);
to prepare the treatment targeted region before delivery of liquid medical substances as vaccines, drugs, antibiotics, medications, mixtures/cocktails of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like. The preparation treatment is done to increase blood circulation via blood vessels dilatation, to modulate inflammation, to create new blood vessels and tissue, and to reduce or eliminate the scarring from the treatment targeted region (see embodiments from FIGS. 15-24B);
to induce the permeabilization of mammalian cells (opening of the cell's membrane vesicles) without destroying a majority of cells in the targeted tissue, which allow the immediate intake of liquid medical substances as vaccines, drugs, antibiotics, medications, mixtures/cocktails of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like. This can significantly accelerate the reaction of the to the injected liquid medical substance and produce a successful treatment (see embodiments from FIGS. 15-25B);
to enhance body absorption, due to formation of new blood vessels and enhanced growth factors, after injection/delivery of liquid medical substances as vaccines, drugs, antibiotics, medications, mixtures/cocktails of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like (see embodiments from FIGS. 15-24B);
to deliver vaccines, drugs, medications, gene medicines, immune cells, antibodies, or stem cells therapeutics (generically called “treatment elements”) that are enveloped or encapsulated in liposomes, lipid nano or micro-particles, or any other artificially or natural envelope, by breaking these envelopes to precisely distribute safely a high dosage of the respective active treatment element to the specific tissue or organ, without side effects in other parts of the body. The size of the envelopes/microparticles used will dictate the type of tissue that is targeted (matching the interstitial or intracellular spaces of that specific tissue or organ), where such enveloped-treatment elements should concentrate. The intact envelopes that did not reached the desired tissue or organ will be safely eliminated via urinary or gastric tracts (see embodiments from FIGS. 15-25B);
to refine the delivery methods that assure the transfer of modified genetic material into the patient's body. While for some of modified genetic material treatments, the actual genetic modification that creates the gene medicines can be done outside the body using CRISPR technology and then inserted into the body, in other cases a Base Editing apparatus and the genetic material will have to be directly inserted into the patient, to efficiently and safely get it to the cells where it needs to do their work. For that, the cells need to open membrane pores and allow the selectively delivery inside them, which can be done by the embodiments presented into these inventions that use shockwaves or pressure waves or ultrasound to accomplish cell membrane-controlled porosity (see embodiments from FIGS. 15-25B);
to allow the use of a combination of an anti-CRISPR protein with an energy-sensitive molecule that can be used as a way to switch the protein on and off. This approach gives the physician a very precise spatial and temporal control of CRISPR gene editing from outside the body. The controlling of anti-CRISPR proteins can be done with small-molecule drugs or by modifying Cas enzymes, which can be activated by using shockwaves or pressure waves or ultrasound or light or other energy means. That allows the control the CRISPR therapies and prevent unnecessary side effects by activating anti-CRISPR selective proteins, which will make the CRISPR treatment more efficient and well targeted for curing a certain genetic disease (see embodiments from FIGS. 15-24B);
to deliver encapsulated stem cells (stem cells in a capsule) that spontaneously self-organize in an in vivo-like 3D conformation or colony promoting fast and homogeneous growth, as well as genomic stability, which when subjected to shockwave or pressure waves or ultrasound directly through the capsule, promote their differentiation into functional microtissues ready for transplantation (see embodiments from FIGS. 15-25B);
to “open” stem cells and neighboring cells' walls/membranes and stimulate inter-cellular communication to facilitate the proper and accelerated stem cell differentiation into the right type of cells and tissues or organs that are needed for the cure or treatment (see embodiments from FIGS. 15-24B);
to introduce gene therapy to a treatment site in conjunction with application of shockwaves or pressure waves or ultrasound to facilitate the gene therapy and subsequently to deliver stem cells to the same treatment site, preferably in combination with applying shockwaves or pressure waves or ultrasound to accelerate differentiation of the stem cells into cells that mimic cells that have been “repaired” by gene therapy (see embodiments from FIGS. 15-25B);
to simultaneously introduce gene therapy and stem cell therapy to a treatment site in conjunction with application of shockwaves or pressure waves or ultrasound to facilitate both genetic modification and stem cell differentiation in the targeted tissue, as an enhanced treatment modality (see embodiments from FIGS. 15-25B);
to introduce stem cell therapy in tissues or organs in conjunction with application of shockwaves or pressure waves or ultrasound to facilitate stem cell differentiation in “healthy” cells necessary to create a proper environment for the surviving of the gene modified cells introduced subsequently via gene therapy, preferably in combination with applying shockwaves or pressure waves or ultrasound, to accelerate absorption of the genetic material used to modify cells, as desired for the treatment (see embodiments from FIGS. 15-25B);
to continue periodically (at least two times per week) the shockwave or pressure waves or ultrasound treatment post-implantation or injection or oral administration or local delivery of the treatment of vaccines, drugs, antibiotics, medications, mixtures/cocktails of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like. This periodically shockwave or pressure waves or ultrasound treatment is done to increase new blood vessels creation (neo-vascularization into the targeted region), which gives enhanced oxygenation and nutrients for tissue or cellular activation and stimulation to promote healing and ultimately functionality regeneration of the tissue or organ (see embodiments from FIGS. 15-24B);
to apply a pure non-contact mechanical action and non-specific to a type of cell or targeted tissue in both temporal or spatial terms, which makes the shockwaves or pressure waves or ultrasound an universal approach for the enhancement of the CRISPR process, or vaccine production, or genetic manipulation, or stem cells proliferation and differentiation, or delivery of vaccines, drugs, antibiotics, medications, mixtures/cocktails of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like (see embodiments from FIGS. 15-25B);
to produce and deliver genetic material, as DNA, RNA, mRNA, gRNA, etc., that can be used for dealing with diseases in animals, or for modifying plants to increase yield and make them more resistant to diseases and parasites. This can create sustainable solutions to address some of the biggest issues facing our planet today, from public health crises to environmentally-friendly food production for a growing population. Also, such agricultural products will help farmers create greener, cleaner crops by precisely targeting a specific pest with non-toxic bio-controls, and without harming beneficial insects or leaving residues in the soil or water (see embodiments from FIGS. 8, 12, 13A-14C and the shockwave or pressure wave or ultrasound devices presented in FIGS. 15-24B);
to apply ex-vivo treatments in combination with stem cells, gene therapies, and other active medical substances, and the like, to generate a desired type of cell, tissue, organ or similar body elements that are subsequently introduced into the body or transplanted (see embodiments from FIGS. 8, 12, 13A-14C and the shockwave or pressure wave or ultrasound devices presented in FIGS. 15-24B);
to produce a gentler stirring of cell cultures or bacterial cultures inside a bioreactor via acoustic streaming and acoustic microstreaming that are pushing particles in preferred directions (unidirectional), which overlap with the longitudinal direction for propagation of the shockwaves or pressure waves or ultrasound. That can effectively and gentle mix the cellular culture or bacterial culture inside the bioreactor and avoids the strong shear forces generated by a paddle system that produces a significant amount of cellular or bacterial death during stirring. By using the shockwaves or pressure waves or ultrasound for stirring the cell cultures or bacterial cultures inside a bioreactor increases the yields for each production batch (see embodiments from FIGS. 8, 12, 13A-14C);
to enhance the filtration or separation processes (both perpendicular or tangential to the filtration membrane surface) to prevent clogging of filtration membranes and enhance the separation of different components during production steps for vaccines, drugs, antibiotics, medications, mixtures/cocktails of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like. This is accomplished via acoustic streaming and microstreaming that are pushing particles in preferred directions (unidirectional), which can expedite the filtration and separation processes and increases the time period in between maintenance cycles and the total number of serviceable cycles, which can generate important savings for the manufacturing facility (see embodiments from FIGS. 15-24B);
to reduce doses, due to facilitated rapid cellular or tissue or organ intake by the shockwaves or pressure waves or ultrasound, for vaccines, drugs, antibiotics, medications, mixtures/cocktails of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like. This translates in more doses produced in one manufacturing batch and also in reduction of the side effects and rejection, which will make the treatment more tolerable and successful. Drugs or medications or vaccines or gene medicines or stem cell therapies, etc. that were rejected for side effects may be brought back into the game due to the enhanced and targeted/localized delivery produced by the shockwaves or pressure waves or ultrasound (see embodiments from FIGS. 15-25B);
to enhance the productivity of manufacturing process or cycle for vaccines, drugs, antibiotics, medications, mixtures/cocktails of multiple active ingredients, gene medicines, stem cells, stem cell cocktails, DNA or RNA/mRNA genetic material, genetic modified material, immune cells (neutrophils, eosinophils, basophils, mast cells, monocytes, macrophages, dendritic cells, natural killer cells, and lymphocyte (B cells and T cells)), antibodies, base editing means, liposomes or lipid nano or micro-particles or any other artificially or natural envelope incorporating active substances, drugs, antibiotics, medication, vaccine material, nano-robots, nano-particles, genetic material, genetic modified material, specific proteins, immune cells, antibodies, base editing means, stem cells, and the like. This can be accomplished by using enhanced processes with shockwaves or pressure waves or ultrasound to get larger outputs (quantity) and in a shorter time-frame (see embodiments from FIGS. 8, 12, 13A-14C).

Important to note is that the shockwave or pressure waves or ultrasound systems do not require moving parts, which increase exponentially their reliability and maintenance simplicity, which overall reduces the cost of the operational costs.

Various embodiments of the invention have been described. It will, however, be evident that various modifications and changes may be made thereto, and additional embodiments may be implemented, without departing from the broader scope of the invention as set forth by the claims. This specification is to be regarded in an illustrative rather than a restrictive sense.

What is claimed is:

1. A method of treating a human or animal body comprising applying pressure waves before or after delivery of an injection of a liquid medical substance and causing a focal volume or pressure field of the pressure waves to completely cover site of the injection and cause rapid uptake of the liquid medical substance in targeted cells by inducing augmented cellular permeability and facilitating stimulation of the targeted cells without killing or destroying a majority of the targeted cells within the focal volume or pressure field, wherein the liquid medical substance is selected from the group consisting of vaccines, drugs, antibiotics, medications, mixtures of multiple active ingredients, cocktails of multiple active ingredients, gene medicines, stem cells, stem cell cocktails, DNA, RNA, mRNA, genetic material, genetically modified material, immune cells, base editors, liposomes, nano-particles, micro-particles, natural envelopes incorporating active substances, artificial envelopes incorporating active substances, nano-robots, and proteins.

2. The method of claim 1, wherein the pressure waves are acoustic pressure shockwaves.

3. The method of claim 2, wherein the shockwaves are focused.

4. The method of claim 2, wherein the shockwaves are concentrated and semi-focused.

5. The method of claim 2, wherein the shockwaves are unfocused.

6. The method of claim 1, wherein the pressure waves are modulated pressure waves.

7. The method of claim 6, wherein the modulated pressure waves are selected from the group consisting of planar, pseudo-planar, radial, and ultrasound waves.

8. The method of claim 7, further comprising stimulating opening of membrane vesicles of the cell via mechanotransduction.

9. The method of claim 2, further comprising stimulating opening of membrane vesicles of the cell via mechanotransduction.

10. The method of claim 1, further comprising applying a membrane of a pressure wave applicator device that contains a fluid within the device against the human or animal body to direct the pressure waves to the tissue.

11. A method of treating a human or animal body comprising administering pressure waves to a tissue to increase cellular permeability within the tissue and cause a focal volume or pressure field of the pressure waves to completely cover an injection site of a treatment agent and cause rapid uptake of the treatment agent in cells of the targeted tissue within the focal volume or pressure field and injecting the treatment agent into the tissue before or after administering the pressure waves.

12. The method of claim 11, wherein the treatment agent is selected from the group consisting of vaccines, drugs, antibiotics, medications, mixtures of multiple active ingredients, cocktails of multiple active ingredients, gene medicines, stem cells, stem cell cocktails, DNA, RNA, mRNA, genetic material, genetically modified material, immune cells, base editors, liposomes, nano-particles, micro-particles, natural envelopes incorporating active substances, artificial envelopes incorporating active substances, nano-robots, and proteins.

13. The method of claim 12, further comprising applying a membrane of a pressure wave applicator device that contains a fluid within the device against the human or animal body to direct the pressure waves to the tissue.

14. The method of claim 13, wherein the pressure waves are acoustic pressure shockwaves.

15. The method of claim 12, wherein the pressure waves are acoustic pressure shockwaves.

16. The method of claim 11, wherein the pressure waves are acoustic pressure shockwaves.

17. The method of claim 16, wherein the acoustic pressure shockwaves are selected from the group consisting of focused, unfocused and semi-focused shockwaves.

18. The method of claim 12, wherein the pressure waves are modulated pressure waves selected from the group consisting of planar, pseudo-planar, radial, and ultrasound waves.

19. The method of claim 11, wherein the pressure waves are modulated pressure waves selected from the group consisting of planar, pseudo-planar, radial, and ultrasound waves.

20. The method of claim 11, further comprising stimulating opening of membrane vesicles of cells in the tissue via mechanotransduction.
